(19) United States
(12) Patent Application Publication
(10) Pub. No.: US 2024/0252483 A1
(43) Pub. Date: Aug. 1, 2024

(54) GASTRIC RESIDENCE SYSTEMS COMPRISING BUPRENORPHINE AND NALOXONE

(71) Applicant: Lyndra Therapeutics, Inc., Watertown, MA (US)

(72) Inventors: Nupura BHISE, Watertown, MA (US); Jeffrey KATSTRA, Watertown, MA (US); David ALTREUTER, Watertown, MA (US)

(21) Appl. No.: 18/289,331

(22) PCT Filed: May 4, 2022
(86) PCT No.: PCT/US2022/072114

§ 371 Date: Nov. 2, 2023

Related U.S. Application Data
(60) Provisional application No. 63/184,734, filed on May 5, 2021.

(51) Int. Cl. A61K 31/485; A61K 9/00; A61K 9/14

(52) U.S. Cl. CPC A61K 31/485; A61K 9/0019; A61K 9/006; A61K 9/0065; A61K 9/146

(57) ABSTRACT

Provided are gastric residence systems comprising at least one co-extruded drug-eluting component comprising a carrier polymer, buprenorphine or a salt thereof, and naloxone or a salt thereof, and a rate-modulating release film coating the at least one co-extruded drug-eluting component. The gastric residence systems comprising at least one co-extruded drug-eluting component are configured to be maintained within a stomach of a human body for at least 48 hours and to release buprenorphine for at least 48 hours, such that the at least one co-extruded drug eluting component with the rate-modulating release film is configured to release at least 10% of the buprenorphine or the salt thereof after the first 24 hours of residence within the stomach and at least 10% of the naloxone or the salt thereof after the first 24 hours of residence within the stomach.

### CROSS-REFERENCE TO RELATED APPLICATIONS

This application claims the benefit of U.S. Provisional Application No. 63/184,734 filed May 5, 2021. The entire contents of that application are hereby incorporated by reference herein.

### STATEMENT REGARDING FEDERALLY SPONSORED RESEARCH OR DEVELOPMENT

This invention was made with government support under Grant No. 5UG3DA047709 awarded by the National Institutes of Health. The government has certain rights in the invention.

### FIELD

The present disclosure relates to gastric residence systems, and more particularly, to gastric residence systems comprising buprenorphine and naloxone.

### BACKGROUND

Buprenorphine is an opioid medication that is used to treat opioid use disorder, acute pain, and chronic pain. When used to treat opioid use disorder, buprenorphine can be administered to a patient when the patient begins experiencing withdrawal symptoms. However, because buprenorphine is addictive, it can be misused by the patient (i.e., overdosed).

Thus, buprenorphine is typically used in conjunction with naloxone. Naloxone is designed to rapidly reverse an opioid overdose. It is an opioid antagonist that binds to opioid receptors and can reverse and block the effects of opioids such as buprenorphine. By combining buprenorphine with naloxone such that the two cannot be separated, the possibility of misusing buprenorphine is greatly decreased. The combination of buprenorphine and naloxone is typically administered sublingually (e.g., film, tablet).

### SUMMARY OF THE DISCLOSURE

Provided herein are gastric residence systems comprising buprenorphine and naloxone. Buprenorphine, an opioid, can cause dependence in users and is subject to mistreatment (e.g., overdosing). Accordingly, buprenorphine is typically administered along with naloxone, which inhibits the effects of opioids and even reverses the effects of an opioid overdose. This combination pharmaceutical composition (e.g., buprenorphine and naloxone) can provide a patient with the therapeutic effects of the buprenorphine without the affects that can lead to dependence and addiction. Thus, by administering buprenorphine with naloxone, the risk of developing a dependence and/or overdosing on buprenorphine is reduced.

However, a challenge posed with buprenorphine/naloxone combination dosage forms is the possibility that a user or patient might intentionally separate the naloxone from the dosage form and just consume the buprenorphine (e.g., a person suffering from opioid use disorder). By doing so, the user will be able to receive the high of the buprenorphine, since the naloxone is no longer present to inhibit such effects. Thus, it is important that dosage forms comprising buprenorphine and naloxone are designed in such a manner to discourage or minimize this possibility.

Accordingly, provided are gastric residence systems comprising buprenorphine and naloxone for use in treating patients having pain or opioid use disorder. Specifically, the gastric residence systems described herein have been formulated to account for and to minimize the possibility of abuse (i.e., removing and consuming only buprenorphine).

To minimize the possibility of abuse, buprenorphine and naloxone may be formulated within a single component of gastric residence systems described herein. As explained below, gastric residence systems can include various components, each with a different function. For example, components of a gastric residence system provided herein can include a central elastomer and two or more elongate arms. Each elongate arm may include time-dependent disintegrating matrices and/or enteric disintegrating matrices. One or more arms of the gastric residence system may also comprise a drug-eluting component. As described herein, to minimize the possibility of buprenorphine and naloxone separation and subsequent mistreatment (i.e., consumption of buprenorphine only), both the buprenorphine and naloxone are provided within the same component of the gastric residence system. Alternatively, the buprenorphine and the naloxone could be formulated separately and provided within separate components of the gastric residence system. However, providing the buprenorphine and naloxone within separate components of the gastric residence system would permit a user to separate the two active ingredients simply by separating the two components from each other, and consume the buprenorphine without the naloxone. Thus, by formulating the buprenorphine and naloxone into a single component (e.g., drug-eluting segment) of a gastric residence system, the two active ingredients are much more difficult to physically separate from each other.

In some embodiments, the buprenorphine and the naloxone are designed to release within a patient's stomach at different rates. For example, the naloxone may release faster than the buprenorphine. In some embodiments, the buprenorphine and naloxone are designed to release in a patient's stomach at a similar rate. If, for example, a user attempted to separate the buprenorphine from the naloxone of the gastric residence system by eluting the naloxone from the gastric residence system outside of the body, the user would be unsuccessful in retrieving the original buprenorphine amount in both gastric residence systems in which the buprenorphine and naloxone have similar release profiles and gastric residence systems in which the buprenorphine and naloxone have different release profiles. In the case in which the buprenorphine and naloxone have similar release profiles, little if any buprenorphine would remain in the gastric residence system after the naloxone completely eluted off. In the case in which the naloxone releases faster than the buprenorphine, a significant amount of the buprenorphine will have eluted during the time taken for all the naloxone to elute from the gastric residence system. Thus, a significantly smaller amount of the original buprenorphine amount would remain. In both cases, the formulation of the buprenorphine and naloxone in a single component of the gastric residence system prevents a user from recovering the entire amount of buprenorphine separately from the naloxone of the gastric residence system, which minimizes the risk of abuse.

The gastric residence systems provided are designed to be swallowed by a patient in a compacted configuration, and, once arriving at the patient's stomach, open to an uncompacted configuration. Once open in the patient's stomach, the gastric residence system will remain in the stomach until the gastric fluids of the stomach cause the gastric residence system to breakdown such that it can pass through the remainder of the patient's gastrointestinal system. The gastric residence systems can comprise one or more linking components (e.g., linkers) linking various components of the gastric residence system together. The linking components comprise a disintegrating matrix and are designed to dissolve or breakdown in the presence of gastric fluids in a controlled manner, allowing various components of the gastric residence system to disassociate and pass through the rest of the patient's gastrointestinal tract. In some embodiments, the gastric residence systems are designed to release the active ingredients (i.e., buprenorphine and naloxone), dissociate, and exit the patient in a controlled amount of time (e.g., 12 hours, 1 day, 2 days, 3 days, 4 days, 5 days, 6 days, 7 days, or 1 month) after the patient first ingests the gastric residence system.

In some embodiments, the gastric residence systems are in the shape of a stellate and comprise a central elastomer, three or more arms, and one or more linking components. The central elastomer of the gastric residence system is the center component of the gastric residence system, from which the three or more arms extend.

The three or more arms of the gastric residence system extend radially outward from the central elastomer of the gastric residence system. One or more of the arms may include the active pharmaceutical ingredients (i.e., buprenorphine and naloxone). The number of active arms (i.e., the one or more arms comprising the active pharmaceutical ingredients) may depend on the dosage amount of the gastric residence system. In some embodiments, the active arms comprise a single buprenorphine/naloxone formulation. In some embodiments, the active arm(s) comprise a combination buprenorphine-naloxone formulation and a naloxone-only formulation co-extruded to form the one or more carrier polymer-agent segment (i.e., drug-eluting segment). The active arms are configured to release the buprenorphine/naloxone formulation(s) once the gastric residence system reaches the patient's stomach.

Accordingly, provided herein are gastric residence systems comprising buprenorphine and naloxone for use in treating patients having pain or opioid use disorder. The gastric residence systems described have been formulated to account for and minimize the possibility of abuse (i.e., removing and consuming only buprenorphine).

In some embodiments, a gastric residence system is provided, the gastric residence system comprising: at least one co-extruded drug-eluting component comprising a carrier polymer, buprenorphine or a salt thereof, and naloxone or a salt thereof; and a rate-modulating release film coating the at least one co-extruded drug-eluting component, wherein the gastric residence system is configured to be maintained within a stomach of a human body for at least 48 hours and to release buprenorphine for at least 48 hours, and the at least one co-extruded drug eluting component with the rate-modulating release film is configured to release at least 10% of the buprenorphine or the salt thereof after the first 24 hours of residence within the stomach and at least 10% of the naloxone or the salt thereof after the first 24 hours of residence within the stomach.

In some embodiments of the gastric residence system, the rate-modulating release film comprises polycaprolactone and copovidone.

In some embodiments of the gastric residence system, the rate-modulating release film comprises 60-90 wt % polycaprolactone.

In some embodiments of the gastric residence system, the rate-modulating release film comprises 10-40 wt % copovidone.

In some embodiments of the gastric residence system, the at least one co-extruded drug-eluting component comprises a first co-extruded portion comprising naloxone and a second co-extruded portion comprising buprenorphine and naloxone.

In some embodiments of the gastric residence system, the first co-extruded portion is embedded in the second co-extruded portion.

In some embodiments of the gastric residence system, the first co-extruded portion comprises one or more strands that are embedded within the second co-extruded portion.

In some embodiments of the gastric residence system, the first co-extruded portion is layered on the second co-extruded portion.

In some embodiments of the gastric residence system, the first co-extruded portion is present at a ratio of 4:1 to the second co-extruded portion.

In some embodiments of the gastric residence system, the first co-extruded portion comprises 35-50 wt % buprenorphine.

In some embodiments of the gastric residence system, the first co-extruded portion comprises 2-7 wt % naloxone.

In some embodiments of the gastric residence system, the first co-extruded portion comprises 35-50 wt % polycaprolactone.

In some embodiments of the gastric residence system, the first co-extruded portion comprises polyethylene glycol and a poloxamer.

In some embodiments of the gastric residence system, the second co-extruded portion comprises 30-50 wt % naloxone.

In some embodiments of the gastric residence system, the second co-extruded portion comprises 50-60 wt % polycaprolactone.

In some embodiments of the gastric residence system, the second co-extruded portion comprises poloxamer.

In some embodiments of the gastric residence system, the at least one co-extruded drug-eluting component comprises 30 mg to 40 mg of buprenorphine or a salt thereof.

In some embodiments of the gastric residence system, the at least one co-extruded drug-eluting component comprises 8 mg to 15 mg of naloxone or a salt thereof.

In some embodiments of the gastric residence system, the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one co-extruded drug-eluting component.

In some embodiments of the gastric residence system, the plurality of arms comprises six arms.

In some embodiments of the gastric residence system, at least two arms of the plurality of arms comprises a co-extruded drug-eluting component of the at least one co-extruded drug-eluting component.

In some embodiments of the gastric residence system, at least three arms of the plurality of arms comprises a co-extruded drug-eluting component of the at least one co-extruded drug-eluting component.

In some embodiments of the gastric residence system, six arms of the plurality of arms comprises a co-extruded drug-eluting component of the at least one co-extruded drug-eluting component.

In some embodiments of the gastric residence system, each arm of the plurality of arms comprises a polymeric linker segment attached to the central elastomer, the polymeric linker segment comprising polycaprolactone.

In some embodiments of the gastric residence system, each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

In some embodiments of the gastric residence system, each arm of the plurality of arms comprises a first inert segment attached to the first disintegrating matrix segment, the first inert segment comprising polycaprolactone, and (BiO)₂CO₃.

In some embodiments of the gastric residence system, each arm of the plurality of arms comprises a second disintegrating matrix segment attached to the first inert segment, the second disintegrating matrix segment comprising polycaprolactone, hydroxypropyl methylcellulose acetate succinate, and a poloxamer.

In some embodiments of the gastric residence system, each arm of the plurality of arms comprises a second inert segment attached to the second disintegrating matrix segment, the second inert segment comprising polycaprolactone, and (BiO)₂CO₃.

In some embodiments of the gastric residence system, a first arm of the plurality of arms comprises a co-extruded drug-eluting component of the at least one co-extruded drug-eluting component attached to the second inert segment.

In some embodiments of the gastric residence system, a second arm of the plurality of arms comprises an inactive segment attached to the second inert segment, the inactive segment comprising polycaprolactone, copovidone, and a poloxamer.

In some embodiments of the gastric residence system, each arm of the plurality of arms comprises a third inert segment attached to one of the co-extruded drug-eluting component or the inactive segment, the third inert segment comprising polycaprolactone, and (BiO)₂CO₃.

In some embodiments of the gastric residence system, each arm of the plurality of arms comprises a third disintegrating matrix segment attached to the third inert segment, the third disintegrating matrix comprising polycaprolactone, hydroxypropyl methylcellulose acetate succinate (HPMCAS); stearic acid; and polypropylene glycol.

In some embodiments of the gastric residence system, the gastric residence system comprises a filament circumferentially connecting each arm, wherein the circumferential filament is attached to the third disintegrating matrix segment of each arm.

In some embodiments, a method of treating an opioid abuse disorder in an individual is provided, the method comprising administering the gastric residence system of to the individual.

In some embodiments, a method of treating pain in an individual is provided, comprising administering the gastric residence system to the individual.

In some embodiments, a gastric residence system is provided, the gastric residence system comprising: at least one drug-eluting component comprising a carrier polymer, buprenorphine or a salt thereof, and naloxone or a salt thereof; and a rate-modulating release film coating the at least one drug-eluting component, wherein the gastric residence system is configured to be maintained within a stomach of a human body for at least 48 hours and to release buprenorphine for at least 48 hours, and the at least one drug-eluting component with the rate-modulating release film is configured to release at least 10% of the buprenorphine or the salt thereof after the first 24 hours of residence within the stomach and at least 10% of the naloxone or the salt thereof within the first 24 hours of residence within the stomach.

In some embodiments of the gastric residence system, the rate-modulating release film comprises polycaprolactone and copovidone.

In some embodiments of the gastric residence system, the rate-modulating release film comprises 60-90 wt % polycaprolactone.

In some embodiments of the gastric residence system, the rate-modulating release film comprises 10-40 wt % copovidone.

In some embodiments of the gastric residence system, the at least one drug-eluting component comprises 35-50 wt % buprenorphine.

In some embodiments of the gastric residence system, the at least one drug-eluting component comprises 2-7 wt % naloxone.

In some embodiments of the gastric residence system, the at least one drug-eluting component comprises 35-50 wt % polycaprolactone.

In some embodiments of the gastric residence system, the at least one drug-eluting component comprises polyethylene glycol and a poloxamer.

In some embodiments of the gastric residence system, the at least one drug-eluting component comprises 10 mg to 30 mg of buprenorphine or a salt thereof.

In some embodiments of the gastric residence system, the at least one drug-eluting component comprises 1 mg to 3 mg of naloxone or a salt thereof.

In some embodiments of the gastric residence system, the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one drug-eluting component.

In some embodiments of the gastric residence system, the plurality of arms comprises six arms.

In some embodiments of the gastric residence system, at least two arms of the plurality of arms comprises a drug-eluting component of the at least one drug-eluting component.

In some embodiments of the gastric residence system, at least three arms of the plurality of arms comprises a drug-eluting component of the at least one drug-eluting component.

In some embodiments of the gastric residence system, six arms of the plurality of arms comprises a drug-eluting component of the at least one drug-eluting component.

In some embodiments of the gastric residence system, each arm of the plurality of arms comprises a polymeric linker segment attached to the central elastomer, the polymeric linker segment comprising polycaprolactone.

In some embodiments of the gastric residence system, each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

In some embodiments of the gastric residence system, each arm of the plurality of arms comprises a first inert segment attached to the first disintegrating matrix segment, the first inert segment comprising polycaprolactone, and (BiO)₂CO₃.

In some embodiments of the gastric residence system, each arm of the plurality of arms comprises a second disintegrating matrix segment attached to the first inert segment, the second disintegrating matrix segment comprising polycaprolactone, hydroxypropyl methylcellulose acetate succinate, and a poloxamer.

In some embodiments of the gastric residence system, each arm of the plurality of arms comprises a second inert segment attached to the second disintegrating matrix segment, the second inert segment comprising polycaprolactone, and (BiO)₂CO₃.

In some embodiments of the gastric residence system, a first arm of the plurality of arms comprises a drug-eluting component of the at least one drug-eluting component attached to the second inert segment.

In some embodiments of the gastric residence system, a second arm of the plurality of arms comprises an inactive segment attached to the second inert segment, the inactive segment comprising polycaprolactone, copovidone, and a poloxamer.

In some embodiments of the gastric residence system, each arm of the plurality of arms comprises a third inert segment attached to one of the drug-eluting component or the inactive segment, the third inert segment comprising polycaprolactone, and (BiO)₂CO₃.

In some embodiments of the gastric residence system, each arm of the plurality of arms comprises a third disintegrating matrix segment attached to the third inert segment, the third disintegrating matrix comprising polycaprolactone, hydroxypropyl methylcellulose acetate succinate (HPMCAS); stearic acid; and polypropylene glycol.

In some embodiments of the gastric residence system, the gastric residence system comprises a filament circumferentially connecting each arm, wherein the circumferential filament is attached to the third disintegrating matrix segment of each arm.

In some embodiments, a method of treating an opioid abuse disorder in an individual is provided, the method comprising administering the gastric residence system of to the individual.

In some embodiments, a method of treating pain in an individual is provided, the method comprising administering the gastric residence system to the individual.

In some embodiments, a gastric residence system is provided, the gastric residence system comprising: a plurality of arms affixed to a central elastomer, wherein at least one arm comprises a drug-eluting component; each arm comprising a proximal end, a distal end, and an outer surface therebetween; wherein the proximal end of each arm is attached to the elastomer component and projects radially from the elastomer component, each arm having its distal end not attached to the elastomer component and located at a larger radial distance from the elastomer component than the proximal end; wherein the at least one arm comprising a drug eluting component comprises: a polymeric linker segment; a first disintegrating matrix segment attached to the polymeric linker segment; a first inert segment attached to the first disintegrating matrix segment; a second disintegrating matrix segment attached to the first inert segment; a second inert segment attached to the second disintegrating matrix segment; the drug-eluting component attached to the second inert segment, wherein the drug eluting component comprises a carrier polymer, buprenorphine or a salt thereof, and naloxone or a salt thereof, and wherein the drug eluting component further comprises a coating comprising a release rate-modulating polymer film; a third inert segment attached to the drug-eluting component; a third disintegrating matrix segment attached to the third inert segment; and a filament circumferentially connecting each arm.

In some embodiments, a method of making a gastric residence system is provided, the method comprising: co-extruding at least one drug-eluting component comprising a carrier polymer, buprenorphine or a salt thereof, and naloxone or a salt thereof; and applying a rate-modulating release film to the at least one co-extruded drug-eluting component, wherein the gastric residence system is configured to be maintained within a stomach of a human body for at least 48 hours and to release buprenorphine for at least 48 hours, and the at least one co-extruded drug eluting component with the rate-modulating release film is configured to release at least 10% of the buprenorphine or the salt thereof after the first 24 hours of residence within the stomach and at least 10% of the naloxone or the salt thereof after the first 24 hours of residence within the stomach.

In some embodiments of the method, the rate-modulating release film comprises polycaprolactone and copovidone.

In some embodiments of the method, the rate-modulating release film comprises 60-90 wt % polycaprolactone.

In some embodiments of the method, the rate-modulating release film comprises 10-40 wt % copovidone.

In some embodiments of the method, the at least one co-extruded drug-eluting component comprises a first co-extruded portion comprising naloxone and a second co-extruded portion comprising buprenorphine and naloxone.

In some embodiments of the method, co-extruding the at least one drug-eluting component comprises co-extruding the first co-extruded portion embedded in the second co-extruded portion.

In some embodiments of the method, co-extruding the at least one drug-eluting component comprises co-extruding strands of the first co-extruded portion embedded within the second co-extruded portion.

In some embodiments of the method, co-extruding the at least one drug-eluting component comprises co-extruding the first co-extruded portion layered on the second co-extruded portion.

In some embodiments of the method, the first co-extruded portion is present at a ratio of 4:1 to the second co-extruded portion.

In some embodiments of the method, the first co-extruded portion comprises 35-50 wt % buprenorphine.

In some embodiments of the method, the first co-extruded portion comprises 2-7 wt % naloxone.

In some embodiments of the method, the first co-extruded portion comprises 35-50 wt % polycaprolactone.

In some embodiments of the method, the first co-extruded portion comprises polyethylene glycol and a poloxamer.

In some embodiments of the method, the second co-extruded portion comprises 30-50 wt % naloxone.

In some embodiments of the method, the second co-extruded portion comprises 50-60 wt % polycaprolactone.

In some embodiments of the method, the second co-extruded portion comprises poloxamer.

In some embodiments of the method, the at least one co-extruded drug-eluting component comprises 30 mg to 40 mg of buprenorphine or a salt thereof.

In some embodiments of the method, the at least one co-extruded drug-eluting component comprises 8 mg to 15 mg of naloxone or a salt thereof.

In some embodiments of the method, the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one co-extruded drug-eluting component.

In some embodiments of the method, the plurality of arms comprises six arms.

In some embodiments of the method, at least two arms of the plurality of arms comprises a co-extruded drug-eluting component of the at least one co-extruded drug-eluting component.

In some embodiments of the method, at least three arms of the plurality of arms comprises a co-extruded drug-eluting component of the at least one co-extruded drug-eluting component.

In some embodiments of the method, six arms of the plurality of arms comprises a co-extruded drug-eluting component of the at least one co-extruded drug-eluting component.

In some embodiments of the method, each arm of the plurality of arms comprises a polymeric linker segment attached to the central elastomer, the polymeric linker segment comprising polycaprolactone.

In some embodiments of the method, each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

In some embodiments of the method, each arm of the plurality of arms comprises a first inert segment attached to the first disintegrating matrix segment, the first inert segment comprising polycaprolactone, and (BiO)₂CO₃.

In some embodiments of the method, each arm of the plurality of arms comprises a second disintegrating matrix segment attached to the first inert segment, the second disintegrating matrix segment comprising polycaprolactone, hydroxypropyl methylcellulose acetate succinate, and a poloxamer.

In some embodiments of the method, each arm of the plurality of arms comprises a second inert segment attached to the second disintegrating matrix segment, the second inert segment comprising polycaprolactone, and (BiO)₂CO₃.

In some embodiments of the method, a first arm of the plurality of arms comprises a co-extruded drug-eluting component of the at least one co-extruded drug-eluting component attached to the second inert segment.

In some embodiments of the method, a second arm of the plurality of arms comprises an inactive segment attached to the second inert segment, the inactive segment comprising polycaprolactone, copovidone, and a poloxamer.

In some embodiments of the method, each arm of the plurality of arms comprises a third inert segment attached to one of the co-extruded drug-eluting component or the inactive segment, the third inert segment comprising polycaprolactone, and (BiO)₂CO₃.

In some embodiments of the method, each arm of the plurality of arms comprises a third disintegrating matrix segment attached to the third inert segment, the third disintegrating matrix comprising polycaprolactone, hydroxypropyl methylcellulose acetate succinate (HPMCAS); stearic acid; and polypropylene glycol.

In some embodiments of the method, the gastric residence system comprises a filament circumferentially connecting each arm, wherein the circumferential filament is attached to the third disintegrating matrix segment of each arm.

In some embodiments, a method of making a gastric residence system is provided, the method comprising: extruding at least one drug-eluting component comprising a carrier polymer, buprenorphine or a salt thereof, and naloxone or a salt thereof; and applying a rate-modulating release film to the at least one drug-eluting component, wherein the gastric residence system is configured to be maintained within a stomach of a human body for at least 48 hours and to release buprenorphine for at least 48 hours, and the at least one drug-eluting component with the rate-modulating release film is configured to release at least 10% of the buprenorphine or the salt thereof after the first 24 hours of residence within the stomach and at least 10% of the naloxone or the salt thereof within the first 24 hours of residence within the stomach.

In some embodiments of the method, the rate-modulating release film comprises polycaprolactone and copovidone.

In some embodiments of the method, the rate-modulating release film comprises 60-90 wt % polycaprolactone.

In some embodiments of the method, the rate-modulating release film comprises 10-40 wt % copovidone.

In some embodiments of the method, the at least one drug-eluting component comprises 35-50 wt % buprenorphine.

In some embodiments of the method, the at least one drug-eluting component comprises 2-7 wt % naloxone.

In some embodiments of the method, the at least one drug-eluting component comprises 35-50 wt % polycaprolactone.

In some embodiments of the method, the at least one drug-eluting component comprises polyethylene glycol and a poloxamer.

In some embodiments of the method, the at least one drug-eluting component comprises 10 mg to 30 mg of buprenorphine or a salt thereof.

In some embodiments of the method, the at least one drug-eluting component comprises 1 mg to 3 mg of naloxone or a salt thereof.

In some embodiments of the method, the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one drug-eluting component.

In some embodiments of the method, the plurality of arms comprises six arms.

In some embodiments of the method, at least two arms of the plurality of arms comprises a drug-eluting component of the at least one drug-eluting component.

In some embodiments of the method, at least three arms of the plurality of arms comprises a drug-eluting component of the at least one drug-eluting component.

In some embodiments of the method, six arms of the plurality of arms comprises a drug-eluting component of the at least one drug-eluting component.

In some embodiments of the method, each arm of the plurality of arms comprises a polymeric linker segment attached to the central elastomer, the polymeric linker segment comprising polycaprolactone.

In some embodiments of the method, each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

In some embodiments of the method, each arm of the plurality of arms comprises a first inert segment attached to the first disintegrating matrix segment, the first inert segment comprising polycaprolactone, and (BiO)₂CO₃.

In some embodiments of the method, each arm of the plurality of arms comprises a second disintegrating matrix segment attached to the first inert segment, the second disintegrating matrix segment comprising polycaprolactone, hydroxypropyl methylcellulose acetate succinate, and a poloxamer.

In some embodiments of the method, each arm of the plurality of arms comprises a second inert segment attached to the second disintegrating matrix segment, the second inert segment comprising polycaprolactone, and (BiO)₂CO₃.

In some embodiments of the method, a first arm of the plurality of arms comprises a drug-eluting component of the at least one drug-eluting component attached to the second inert segment.

In some embodiments of the method, a second arm of the plurality of arms comprises an inactive segment attached to the second inert segment, the inactive segment comprising polycaprolactone, copovidone, and a poloxamer.

In some embodiments of the method, each arm of the plurality of arms comprises a third inert segment attached to one of the drug-eluting component or the inactive segment, the third inert segment comprising polycaprolactone, and (BiO)₂CO₃.

In some embodiments of the method, each arm of the plurality of arms comprises a third disintegrating matrix segment attached to the third inert segment, the third disintegrating matrix comprising polycaprolactone, hydroxypropyl methylcellulose acetate succinate (HPMCAS); stearic acid; and polypropylene glycol.

In some embodiments of the method, the gastric residence system comprises a filament circumferentially connecting each arm, wherein the circumferential filament is attached to the third disintegrating matrix segment of each arm.

In some embodiments, any one or more of the features, characteristics, or elements discussed above with respect to any of the embodiments may be incorporated into any of the other embodiments mentioned above or described elsewhere herein.

### BRIEF DESCRIPTION OF THE FIGURES

This application contains at least one drawing executed in color. Copies of this patent application publication with color drawing(s) will be provided by the Office upon request and payment of the necessary fee.

FIG. 1A shows a gastric residence system having a stellate configuration, according to some embodiments;

FIG. 1B shows a gastric residence system in a folded configuration, according to some embodiments;

FIG. 2A shows an example co-extrusion geometry for a drug-eluting segment, according to some embodiments;

FIG. 2B shows an example co-extrusion geometry for a drug-eluting segment, according to some embodiments;

FIG. 3A shows a gastric residence system having six active arms, according to some embodiments;

FIG. 3B shows a gastric residence system having four active arms, according to some embodiments;

FIG. 3C shows a gastric residence system having two active arms, according to some embodiments;

FIG. 4A shows BUP release profiles for coated and uncoated drug-eluting segments comprising a BUP-NAL combination formulation;

FIG. 4B shows NAL release profiles for coated and uncoated drug-eluting segments comprising a BUP-NAL combination formulation;

FIG. 5A shows release profiles for a gastric residence system comprising a buprenorphine-naloxone combination formulation, according to some embodiments;

FIG. 5B shows release profile for a gastric residence system comprising a naloxone-only formulation, according to some embodiments;

FIG. 6A shows release profiles for gastric residence systems comprising drug-eluting segments co-extruded with a BUP-NAL combination formulation and a NAL-only formulation, according to some embodiments;

FIG. 6B shows BUP:NAL mass ratios over the course of a seven-day period from the time of administration for a gastric residence system comprising drug-eluting segments co-extruded with a BUP-NAL combination formulation and a NAL-only formulation, according to some embodiments;

FIG. 7A shows mean plasma buprenorphine concentrations in dog plasma after dosing with BUP sublingually;

FIG. 7B shows mean plasma buprenorphine concentrations in dog plasma after dosing with a gastric residence system comprising BUP-NAL;

FIG. 7C shows mean plasma concentrations of norbuprenorphine in dog plasma after dosing with a gastric residence system comprises BUP-NAL;

FIG. 8A shows mean buprenorphine plasma concentrations in cynomolgous monkeys after dosing with BUP intravenously;

FIG. 8B shows mean buprenorphine plasma concentrations in cynomolgous monkeys after dosing with a gastric residence system comprising BUP-NAL; and

FIG. 8C shows mean norbuprenorphine plasma concentrations in cynomolgous monkeys after dosing with a gastric residence system comprising BUP-NAL.

### DETAILED DESCRIPTION OF THE DISCLOSURE

Described herein are gastric residence systems comprising buprenorphine and naloxone for treating pain or opioid use disorder. Specifically, the gastric residence systems described are designed to minimize the possibility of buprenorphine abuse. This can be achieved by providing a dosage form comprising buprenorphine and naloxone from which the buprenorphine and/or naloxone cannot be extracted and the buprenorphine individually consumed.

The gastric residence systems for administering the buprenorphine and naloxone may be in the shape of a stellate. For example, the stellate-shaped gastric residence systems may include a central elastomer, three or more arms, and a plurality of linking components (i.e., linkers). The central elastomer is located at the center of the stellate, and the three or more arms may extend radially from the central elastomer. The linkers may couple two or more components of the gastric residence systems together. For example, a linker may connect an arm to the central elastomer. A linker might also connect two lengths of an arm together (e.g., act as an elbow of the arm). In some embodiments, the active pharmaceutical ingredients (i.e., the buprenorphine and the naloxone) may be provided in an arm of the stellate (i.e., an active arm). The active arm(s) may include a single buprenorphine-naloxone formulation.

In some embodiments, the active arm(s) may include two separate formulations—a buprenorphine-naloxone combination formulation and a naloxone-only formulation—coextruded together to form an associated buprenorphine and naloxone formulation. In either case (i.e., the buprenorphine-naloxone formulation or the associated formulation comprising a buprenorphine-naloxone combination formulation coextruded with a naloxone-only formulation), the possibility of a user being able to remove the naloxone from the dosage form and just consuming the buprenorphine is slight.

Described below are gastric residence systems for administering buprenorphine and naloxone to a patient. Specifically, the discussion below provides: (1) definitions; (2) gastric residence system drug delivery mechanism; (3) configuration and components of gastric residence systems; (4) features for improved retention and agent release; (5) carrier polymer-agent segments comprising a combination BUP-NAL formulation; (6) carrier polymer-agent segments formed by co-extruding a combination BUP-NAL formulation with a NAL-only formulation; (7) rate-modulating polymer films; (8) gastric residence time; (9) gastric residence systems comprising BUP and NAL; and (10) examples.

### Definitions

A “carrier polymer” is a polymer suitable for blending with an agent, such as a drug, for use in a gastric residence system.

An “agent” is any substance intended for therapeutic, diagnostic, or nutritional use in a patient, individual, or subject. Agents include, but are not limited to, drugs, nutrients, vitamins, and minerals.

A “dispersant” is defined as a substance which aids in the minimization of particle size of agent and the dispersal of agent particles in the carrier polymer matrix. That is, the dispersant helps minimize or prevent aggregation or flocculation of particles during fabrication of the systems. Thus, the dispersant has anti-aggregant activity and anti-flocculant activity, and helps maintain an even distribution of agent particles in the carrier polymer matrix.

An “excipient” is any substance added to a formulation of an agent that is not the agent itself. Excipients include, but are not limited to, binders, coatings, diluents, disintegrants, emulsifiers, flavorings, glidants, lubricants, and preservatives. The specific category of dispersant falls within the more general category of excipient.

An “elastic polymer” or “elastomer” is a polymer that is capable of being deformed by an applied force from its original shape for a period of time, and which then substantially returns to its original shape once the applied force is removed.

A “patient,” “individual,” or “subject” refers to a mammal, preferably a human or a domestic animal such as a dog or cat. In a most preferred embodiment, a patient, individual, or subject is a human.

The “diameter” of a particle as used herein refers to the longest dimension of a particle.

A “poloxamer” is a block co-polymer having a central polypropylene oxide core, with a flanking region of polyethylene oxide on either side of the core.

“Treating” a disease or disorder with the systems and methods disclosed herein is defined as administering one or more of the systems disclosed herein to a patient in need thereof, with or without additional agents, in order to reduce or eliminate either the disease or disorder, or one or more symptoms of the disease or disorder, or to retard the progression of the disease or disorder or of one or more symptoms of the disease or disorder, or to reduce the severity of the disease or disorder or of one or more symptoms of the disease or disorder. “Suppression” of a disease or disorder with the systems and methods disclosed herein is defined as administering one or more of the systems disclosed herein to a patient in need thereof, with or without additional agents, in order to inhibit the clinical manifestation of the disease or disorder, or to inhibit the manifestation of adverse symptoms of the disease or disorder. The distinction between treatment and suppression is that treatment occurs after adverse symptoms of the disease or disorder are manifest in a patient, while suppression occurs before adverse symptoms of the disease or disorder are manifest in a patient. Suppression may be partial, substantially total, or total. Because some diseases or disorders are inherited, genetic screening can be used to identify patients at risk of the disease or disorder. The systems and methods disclosed herein can then be used to treat asymptomatic patients at risk of developing the clinical symptoms of the disease or disorder, in order to suppress the appearance of any adverse symptoms.

A “flexural modulus” of a material is an intrinsic property of a material computed as the ratio of stress to strain in flexural deformation of the material as measured by a 3-point bending test. Although the linkers are described herein as being components of the gastric residence system, the flexural modulus of the material of the polymeric material may be measured in isolation. For example, the polymeric linker in the gastric residence system may be too short to measure the flexural modulus, but a longer sample of the same material may be used to accurately determine the flexural modulus. The longer sample used to measure the flexural modulus should have the same cross-sectional dimensions (shape and size) as the polymeric linker used in the gastric residence system. The flexural modulus is measured using a 3-point bending test in accordance with the ASTM standard 3-point bending test (ASTM D790) using a 10 mm distance between supports and further modified to accommodate materials with non-rectangular cross-sections. The longest line of symmetry for the cross section of the polymeric linker should be positioned vertically, and the flexural modulus should be measured by applying force downward. If the longest line of symmetry for the cross section of the polymeric linker is perpendicular to a single flat edge, the single flat edge should be positioned upward. If the cross-section of the polymeric linker is triangular, the apex of the triangle should be faced downward. As force is applied downward, force and displacement are measured, and the slope at the linear region is obtained to calculate the flexural modulus.

As used herein, the singular forms “a”, “an”, and “the” include plural references unless indicated otherwise or the context clearly dictates otherwise.

When numerical values are expressed herein using the term “about” or the term “approximately,” it is understood that both the value specified, as well as values reasonably close to the value specified, are included. For example, the description “about 50° C.” or “approximately 50° C.” includes both the disclosure of 50° C. itself, as well as values close to 50° C. Thus, the phrases “about X” or “approximately X” include a description of the value X itself. If a range is indicated, such as “approximately 50° C. to 60° C.” or “about 50° C. to 60° C.,” it is understood that both the values specified by the endpoints are included, and that values close to each endpoint or both endpoints are included for each endpoint or both endpoints; that is, “approximately 50° C. to 60° C.” (or “about 50° C. to 60° C.”) is equivalent to reciting both “50° C. to 60° C.” and “approximately 50° C. to approximately 60° C.” (or “about 50° C. to 60° C.”).

With respect to numerical ranges disclosed in the present description, any disclosed upper limit for a component may be combined with any disclosed lower limit for that component to provide a range (provided that the upper limit is greater than the lower limit with which it is to be combined). Each of these combinations of disclosed upper and lower limits are explicitly envisaged herein. For example, if ranges for the amount of a particular component are given as 10% to 30%, 10% to 12%, and 15% to 20%, the ranges 10% to 20% and 15% to 30% are also envisaged, whereas the combination of a 15% lower limit and a 12% upper limit is not possible and hence is not envisaged.

Unless otherwise specified, percentages of ingredients in compositions are expressed as weight percent, or weight/weight percent. It is understood that reference to relative weight percentages in a composition assumes that the combined total weight percentages of all components in the composition add up to 100. It is further understood that relative weight percentages of one or more components may be adjusted upwards or downwards such that the weight percent of the components in the composition combine to a total of 100, provided that the weight percent of any particular component does not fall outside the limits of the range specified for that component.

Some embodiments described herein are recited as “comprising” or “comprises” with respect to their various elements. In alternative embodiments, those elements can be recited with the transitional phrase “consisting essentially of” or “consists essentially of” as applied to those elements. In further alternative embodiments, those elements can be recited with the transitional phrase “consisting of” or “consists of” as applied to those elements. Thus, for example, if a composition or method is disclosed herein as comprising A and B, the alternative embodiment for that composition or method of “consisting essentially of A and B” and the alternative embodiment for that composition or method of “consisting of A and B” are also considered to have been disclosed herein. Likewise, embodiments recited as “consisting essentially of” or “consisting of” with respect to their various elements can also be recited as “comprising” as applied to those elements. Finally, embodiments recited as “consisting essentially of” with respect to their various elements can also be recited as “consisting of” as applied to those elements, and embodiments recited as “consisting of” with respect to their various elements can also be recited as “consisting essentially of” as applied to those elements.

When a composition or system is described as “consisting essentially of” the listed elements, the composition or system contains the elements expressly listed, and may contain other elements which do not materially affect the condition being treated (for compositions for treating conditions), or the properties of the described system (for compositions comprising a system). However, the composition or system either does not contain any other elements which do materially affect the condition being treated other than those elements expressly listed (for compositions for treating systems) or does not contain any other elements which do materially affect the properties of the system (for compositions comprising a system); or, if the composition or system does contain extra elements other than those listed which may materially affect the condition being treated or the properties of the system, the composition or system does not contain a sufficient concentration or amount of those extra elements to materially affect the condition being treated or the properties of the system. When a method is described as “consisting essentially of” the listed steps, the method contains the steps listed, and may contain other steps that do not materially affect the condition being treated by the method or the properties of the system produced by the method, but the method does not contain any other steps which materially affect the condition being treated or the system produced other than those steps expressly listed.

This disclosure provides several embodiments. It is contemplated that any features from any embodiment can be combined with any features from any other embodiment where possible. In this fashion, hybrid configurations of the disclosed features are within the scope of the present disclosure.

In addition to the embodiments and methods disclosed here, additional embodiments of gastric residence systems, and methods of making and using such systems, are disclosed in International Patent Application Nos. WO 2015/191920, WO 2015/191925, WO 2017/070612, WO 2017/100367, and PCT/US2017/034856, which are incorporated by reference herein in their entirety.

The following abbreviations for polymers and other components are used:

| Abbreviation | Component |
|---|---|
| PDL | poly(DL-lactide); inherent viscosity 1.6-2.4 dl/g (CHCl₃), Tₘ |
| | 165-180° C. |
| PCL | polycaprolactone |
| VA64 | copovidone; Tₘ 140° C., T_{g} 101° C. |
| Kollidon SR | Polyvinyl acetate/polyvinylpyrrolidone |
| PEO_{100K} | polyethylene glycol; MW (ave) 100,000 |
| PPG | polypropylene glycol |
| PDLG | copolymer of DL-lactide and glycolide); inherent viscosity 1.6- |
| | 2.4 dl/g (CHCl₃) |
| Corbion PC17 | PURASORB ® Polycaprolactone; GMP grade homopolymer of ε- |
| | Caprolactone with an inherent viscosity midpoint of 1.7 dl/g |
| Corbion PC04 | PURASORB ® Polycaprolactone; GMP grade homopolymer of ε- |
| | Caprolactone with an inherent viscosity midpoint of 0.4 dl/g |
| PG | propylene glycol |
| Corbion | PURASORB ® 50/50 DL-lactide/glycolide copolymer; GMP |
| 5004/PURASORB ® | grade copolymer of DL-lactide and Glycolide in a 50/50 molar |
| PDLG 5004 | ratio and with an inherent viscosity midpoint of 0.4 dl/g |
| Corbion 5004A/ | PURASORB ® 50/50 DL-lactide/glycolide copolymer; acid |
| PURASORB ® | terminated GMP grade copolymer of DL-lactide and Glycolide in |
| PDLG 5004A | a 50/50 molar ratio and with an inherent viscosity midpoint of 0.4 |
| | dl/g |
| HPMCAS | Synthetic polymer derived from cellulose |
| P407 | Poloxamer 407; PEG-PPG-PEG triblock co-polymer |
| E172 | Ferrosoferric Oxide |

PLURONIC® is a registered trademark of BASF Corporation for polyoxyalkylene ethers. In any formulation described herein using trade names, the trade name can be replaced by the generic name. For example, a formulation described as comprising 50% Corbion PC17 and 50% Corbion PC04 is understood to describe a formulation comprising 50% polycaprolactone of viscosity 1.7 dl/g and 50% polycaprolactone of viscosity 0.4 dl/g.

### Gastric Residence System Drug Delivery Mechanism

Gastric residence dosage forms can be designed to be administered to the stomach of a patient by swallowing, by feeding tube, by gastric tube, etc. In some embodiments, the gastric residence dosage forms are folded into a compacted configuration and secured in a capsule. Once a gastric residence dosage form is in place in the stomach, it can open from its compacted form to an uncompacted form and remain in the stomach for a desired residence time (e.g., three days, seven days, two weeks, etc.). A gastric residence dosage form that is properly in place in a stomach will resist passage through the pyloric valve, which separates the stomach from the small intestine. Gastric residence dosage forms can release a therapeutic agent (i.e., API or drug) over the period of residence with controlled release. While residing in the stomach, the dosage form may not interfere with the normal passage of food or other gastric contents. Once the desired residence time has expired, the dosage form passes out of the stomach (i.e., through the pyloric valve) and is readily eliminated from the patient.

To administer a gastric residence system to a patient, the gastric residence system can be folded into a form small enough to be swallowed or otherwise administered. In some embodiments, the folded gastric residence system is retained in a capsule or other container which can be swallowed by the patient. In some cases, the gastric residence system may be delivered to a patient via gastrostomy tube, feeding tube, gastric tube, or other route of administration to the stomach. Specific examples of gastric residence systems may be found in PCT/US2018/051816, WO 2015/191920, WO 2017/070612, WO 2017/100367, WO 2018/064630, WO 2017/205844, WO 2018/227147, each of which is incorporated herein in its entirety.

Once the gastric residence system reaches the stomach of a patient, it may assume an open configuration. The dimensions of an open gastric residence system are, when left unaltered, suitable to prevent passage of the device through the pyloric valve for the period of time during which the device is intended to reside in the stomach. In some embodiments, the folded gastric residence system can also be secured by a dissolvable retaining band or sleeve that can prevent premature deployment of the gastric residence system in case of a failure of the capsule.

While in the stomach, the gastric residence system is compatible with digestion and other normal functioning of the stomach or gastrointestinal tract. The gastric residence system does not interfere with or impede the passage of chyme (partially digested food) or other gastric contents which exit the stomach through the pyloric valve into the duodenum.

Once released from the capsule into the stomach, the therapeutic agent (e.g., buprenorphine, naloxone) of the gastric residence system begins to take effect. In some embodiments, the gastric residence system comprises a plurality of carrier polymer-agent components. The carrier polymer-agent components may comprise a carrier polymer, a pore former, and a therapeutic agent (or a salt thereof). The plurality of carrier polymer-agent components are linked together by one or more coupling polymer components. The therapeutic agent may be eluted from the carrier polymer-agent components into the gastric fluid of the patient over the desired residence time of the system. Release of the therapeutic agent is controlled by appropriate formulation of the carrier polymer-agent components, including by the use of the dispersant in formulation of the carrier polymer-agent components, and by milling of the therapeutic agent to particles of desired size prior to blending the agent with the carrier polymer and dispersant.

Additionally, coatings can be applied to outer surfaces of the gastric residence system. The coatings can include additional therapeutic agents or agents that can affect the release of therapeutic agents or the residence duration of the gastric residence system.

Once the desired residence time has expired, the gastric residence system passes out of the stomach. To do so, various components of the gastric delivery system are designed to weaken and degrade. The specific dimensions of the system are also taken into consideration. In its intact, uncompacted, open configuration, the gastric residence system is designed to resist passage through the pyloric valve. However, coupling polymer components of the gastric residence system are chosen such that they gradually degrade over the specified residence period in the stomach. When the coupling polymer components are sufficiently weakened by degradation, the gastric residence system loses critical resilience to compression or size reduction and can break apart into smaller pieces. The reduced-size dosage form and any smaller pieces are designed to pass through the pyloric valve. The system then passes through the intestines and is eliminated from the patient. In some embodiments, a gastric residence system may be designed to weaken at specific locations such that the gastric residence system can pass through a pyloric valve intact once the residence time expires without degrading into numerous smaller pieces.

### Configuration & Components of Gastric Residence System

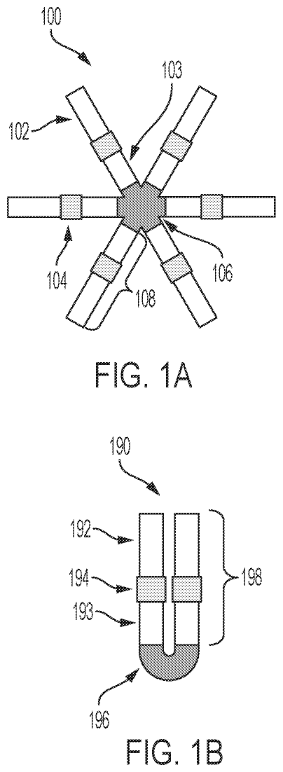

Gastric residence systems can be prepared in different configurations. The “stellate” configuration of a gastric residence system is also known as a “star” (or “asterisk”) configuration. An example of a stellate system 100 is shown schematically in FIG. 1A. Multiple arms (only one such arm, 108, is labeled for clarity), are affixed to disk-shaped central elastomer 106. The arms depicted in FIG. 1A are comprised of segments 102 and 103, joined by a coupling polymer or linker region 104 (again, the components are only labeled in one arm for clarity) which serves as a linker region. This configuration permits the system to be folded or compacted at the central elastomer. FIG. 1B shows a folded configuration 190 of the gastric residence system of FIG. 1A (for clarity, only two arms are illustrated in FIG. 1B). Segments 192 and 193, linker region 194, elastomer 196, and arm 198 of FIG. 1B correspond to segments 102 and 103, linker region 104, elastomer 106, and arm 108 of FIG. 1A, respectively. When folded, the overall length of the system is reduced by approximately a factor of two, and the system can be conveniently placed in a container such as a capsule or other container suitable for oral administration. The gastric residence system is constrained by the capsule or other container into the compacted state (the folded state). When the capsule reaches the stomach, the capsule dissolves, releasing the gastric residence system. Upon release of the constraint by the capsule or other container, the gastric residence system then unfolds into its uncompacted state, which is retained in the stomach for the desired residence period.

While the linker regions 104 are shown as slightly larger in diameter than the segments 102 and 103 in FIG. 1A, they can be the same diameter as the segments, so that the entire arm 102-104-103 has a smooth outer surface.

In some embodiments, the stellate system may have an arm composed of only one segment, which is attached to the central elastomer by a linker region. This corresponds to FIG. 1A with the segments 103 omitted. The single-segment arms comprising segments 102 are then directly attached to central elastomer 106 via the linkers 104. The linkers can comprise a coupling polymer or a disintegrating matrix.

A stellate system can be described as a gastric residence system for administration to the stomach of a patient, comprising an elastomer component, and at least one carrier polymer-agent component comprising a carrier polymer and an agent or a salt thereof, attached to the elastomer component, wherein each of the plurality of carrier polymer-agent components is an arm comprising a proximal end, a distal end, and an outer surface therebetween; wherein the proximal end of each arm is attached to the elastomer component and projects radially from the elastomer component, each arm having its distal end not attached to the elastomer component and located at a larger radial distance from the elastomer component than the proximal end; wherein each arm independently comprises one or more segments, each segment comprising a proximal end, a distal end, and an outer surface therebetween. In some embodiments, when two or more segments are present in an arm, each segment is attached to an adjacent segment via a linker region. In some embodiments, when two or more segments are present in an arm, one segment is directly attached to the other segment, without using a linker region. The linker region can be a coupling polymer or a disintegrating matrix. The arms can be attached to the central elastomer via a coupling polymer or a disintegrating matrix, and can have intervening portions of interfacing polymers. For the plurality of at least three arms, or for a plurality of arms, a preferred number of arms is six, but three, four, five, seven, eight, nine, or ten arms can be used. The arms should be equally spaced around the central elastomer; if there are N arms, there will be an angle of about 360/N degrees between neighboring arms.

### Coupling Polymers/Linker Regions

The coupling polymers of the gastric residence system, which serve as linker regions, are designed to break down gradually in a controlled manner during the residence period of the system in the stomach. If the gastric residence system passes prematurely into the small intestine in an intact form, the system is designed to break down much more rapidly to avoid intestinal obstruction. This is readily accomplished by using enteric polymers as coupling polymers. Enteric polymers are relatively resistant to the acidic pH levels encountered in the stomach, but dissolve at the higher pH levels found in the duodenum. Use of enteric coupling polymers as safety elements protects against undesired passage of the intact gastric residence system into the small intestine. In the system shown in FIG. 1A, at least the coupling polymer used for the couplings 104 are made from such enteric polymers.

In additional embodiments, a time-dependent coupling polymer or linker can be used. Such a time-dependent coupling polymer or linker degrades in a predictable, time-dependent manner. In some embodiments, the degradation of the time-dependent coupling polymer or linker may not be affected by the varying pH of the gastrointestinal system.

In additional embodiments, different types of linkers can be used in the gastric residence systems. That is, both enteric linkers (or enteric coupling polymers) and time-dependent linkers (or time-dependent coupling polymers) can be used. In some embodiments, a single multi-segment arm of a stellate system can use both an enteric linker at some linker regions between segments, and a time-dependent linker at other linker regions between segments.

Linker regions are typically about 100 microns to about 2 millimeter in width, such as about 200 um to about 2000 um, about 300 um to about 2000 um, about 400 um to about 2000 um, about 500 um to about 2000 um, about 600 um to about 2000 um, about 700 um to about 2000 um, about 800 um to about 2000 um, about 900 um to about 2000 um, about 1000 um to about 2000 um, about 1100 um to about 2000 um, about 1200 um to about 2000 um, about 1300 um to about 2000 um, about 1400 um to about 2000 um, about 1500 um to about 2000 um, about 1600 um to about 2000 um, about 1700 um to about 2000 um, about 1800 um to about 2000 um, or about 1900 um to about 2000 um; or about 100 um to about 1900 um, about 100 um to about 1800 um, about 100 um to about 1700 um, about 100 um to about 1600 um, about 100 um to about 1500 um, about 100 um to about 1400 um, about 100 to about 1300 um, about 100 um to about 1200 um, about 100 um to about 1100 um, about 100 um to about 1000 um, about 100 um to about 900 um, about 100 um to about 800 um, about 100 um to about 700 um, about 100 um to about 600 um, about 100 um to about 500 um, about 100 um to about 400 um, about 100 um to about 300 um, or about 100 um to about 200 um. Linker regions can be about 100 um, about 200 um, about 300 um, about 400 um, about 500 um, about 600 um, about 700 um, about 800 um, about 900 um, about 1000 um, about 1100 um, about 1200 um, about 1300 um, about 1400 um, about 1500 um, about 1600 um, about 1700 um, about 1800 um, about 1900 um, or about 2000 um in width, where each value can be plus or minus 50 um (+50 um).

### Central Elastomer

The central elastomeric polymer of a stellate system is typically not an enteric polymer; however, the central elastomeric polymer can also be made from such an enteric polymer where desirable and practical.

The central elastomer should have a specific durometer and compression set. The durometer is important because it determines the folding force of the dosage form and whether it will remain in the stomach; a preferred range is from about 60 to about 90 A. The compression set should be as low as possible to avoid having permanent deformation of the gastric residence system when stored in the capsule in its compacted configuration. A preferred range is about 10% to about 20% range. Liquid silicone rubber is a useful material for the central elastomer. Examples of materials that fit these requirements are the QP1 range of liquid silicone rubbers from Dow Corning. In any embodiment with a central elastomer, the QP1-270 (70 A durometer) liquid silicone rubber can be used. In some embodiments, the central elastomer may comprise a 50 A or 60 A durometer liquid silicone rubber (Shin Etsu).

### Segments/Arms

Segments and arms of the gastric residence systems can have cross-sections in the shape of a circle (in which case the segments are cylindrical), a polygon (such as segments with a triangular cross-section, rectangular cross-section, or square cross-section), or a pie-shaped cross-section (in which case the segments are cylindrical sections). Segments with polygon-shaped or pie-shaped cross-sections, and ends of cylindrically-shaped sections which will come into contact with gastric tissue, can have their sharp edges rounded off to provide rounded corners and edges, for enhanced safety in vivo. That is, instead of having a sharp transition between intersecting edges or planes, an arc is used to transition from one edge or plane to another edge or plane. Thus, a “triangular cross-section” includes cross-sections with an approximately triangular shape, such as a triangle with rounded corners. An arm with a triangular cross-section includes an arm where the edges are rounded, and the corners at the end of the arm are rounded. Rounded corners and edges are also referred to as fillet corners, filleted corners, fillet edges, or filleted edges.

In some embodiments, the stellate system is about 30 mm to about 60 mm when unfolded (arm extended). In some embodiments, the stellate system is about 41 mm to about 51 mm when unfolded. In some embodiments, the stellate system is about 45 mm to about 47 mm when unfolded. In some embodiments, the stellate system is about 46 mm when unfolded.

### Features for Improved Retention and Agent Release for Buprenorphine/Naloxone Gastric Residence Systems

Retention of gastric residence systems for the desired residence period and agent release from gastric residence systems can be improved and made more consistent using the features described herein, such as a filament which is wrapped circumferentially around a gastric residence system and connecting the arms of the gastric residence system; use of timed linkers and enteric linkers which permit higher precision in retention and passage of the gastric residence system; and arms coated with release rate-modulating polymer films.

### Circumferential Filament

Gastric residence systems having a filament described herein may help improve the gastric residence of the gastric residence system. Specifically, a filament can help provide a more consistent gastric residence time and/or a longer gastric residence time. Thus, gastric residence systems provided herein that include a filament may provide more predictable and/or controllable gastric residence times. Gastric residence systems having predictable and/or controllable gastric residence times can minimize the risk of the gastric residence system unfolding too early (e.g., in the esophagus) and causing an obstruction. Gastric residence systems having predictable and/or controllable gastric residence times can also minimize the possibility of the gastric residence system passing through the stomach and unfolding later in the gastrointestinal tract (i.e., intestine), or passing through the gastrointestinal tract without unfolding at all. In each of these possible scenarios, the therapeutic agent of the gastric residence dosage form is not delivered to the patient as intended.

However, it has been demonstrated that gastric residence systems of a stellate shape can bend into a configuration that allows for premature passage through the pylorus of a patient. Gastric residence systems that prematurely pass through the pylorus fail to deliver the therapeutic agent of the gastric residence system to the patient. Further, premature passage causes inconsistency, causes unreliability, and compromises the efficacy of the gastric residence system.

The feature of circumferential filament is described in International Patent Application PCT/US2020/059541, which is hereby incorporated by reference in its entirety.

### Time-Dependent Linkers (Timed Disintegrating Matrices) and Enteric Linkers (Enteric Disintegrating Matrices)

Described below are polymeric linkers (e.g., timed linkers and/or enteric linkers) and times-dependent linkers specifically.

### Polymeric Linkers

The agent-containing structural members are attached to a second structural member (such as a central member, which may be an elastic central member) through one or more linkers. A polymeric linker may directly interface with the agent-containing structural member, or may interface with the agent-containing structural member through a coupling member. Similarly, the polymeric linker may interface directly with the second structural member, or may interface through a coupling member. In an embodiment wherein the agent-containing structural member is connected to the second structural member through two or more polymeric linkers, the polymeric linkers may directly interface with each other, or may interface through a coupling member. One or both of an enteric linker and a time-dependent linkers may be used, or a polymeric linker may function as both an enteric linker and a time-dependent linker.

The polymeric linkers are typically about 100 microns to about 3 millimeter in width, such as about 200 um to about 3000 um, about 300 um to about 3000 um, about 400 um to about 3000 um, about 500 um to about 3000 um, about 600 um to about 3000 um, about 700 um to about 3000 um, about 800 um to about 3000 um, about 900 um to about 3000 um, about 1000 um to about 3000 um, about 1100 um to about 3000 um, about 1200 um to about 3000 um, about 1300 um to about 3000 um, about 1400 um to about 3000 um, about 1500 um to about 3000 um, about 1600 um to about 3000 um, about 1700 um to about 3000 um, about 1800 um to about 3000 um, about 1900 um to about 3000 um, about 2000 um to about 3000 um, about 2100 um to about 3000 um, about 2200 um to about 3000 um, about 2300 um to about 3000 um, about 2400 um to about 3000 um, about 2500 um to about 3000 um, about 2600 um to about 3000 um, about 2700 um to about 3000 um, about 2800 um to about 3000 um, or about 2900 um to about 3000 um; or about 100 um to about 200 um, about 200 um to about 300 um, about 300 um to about 400 um, about 400 um to about 500 um, about 500 um to about 600 um, about 600 um to about 700 um, about 700 um to about 800 um, about 800 um to about 900 um, about 900 um to about 1000 um, about 1000 um to about 1100 um, about 1100 um to about 1200 um, about 1200 um to about 1300 um, about 1300 um to about 1400 um, about 1400 um to about 1500 um, about 1500 um to about 1600 um, about 1600 um to about 1700 um, about 1700 um to about 1800 um, about 1800 um to about 1900 um, about 1900 um to about 2000 um, about 2000 um to about 2100 um, about 2100 um to about 2200 um, about 2200 um to about 2300 um, about 2300 um to about 2400 um, about 2400 um to about 2500 um, about 2500 um to about 2600 um, about 2600 um to about 2700 um, about 2700 um to about 2800 um, about 2800 um to about 2900 um, about 2900 um to about 3000 um. Polymeric linkers can be about 100 um, about 200 um, about 300 um, about 400 um, about 500 um, about 600 um, about 700 um, about 800 um, about 900 um, about 1000 um, about 1100 um, about 1200 um, about 1300 um, about 1400 um, about 1500 um, about 1600 um, about 1700 um, about 1800 um, about 1900 um, about 2000 um, about 2100 um, about 2200 um, about 2300 um, about 2400 um, about 2500 um, about 2600 um, about 2700 um, about 2800 um, about 2900 um, about 3000 um in width, where each value can be plus or minus 50 um (±50 um).

The cross section of the polymeric linker may be round (i.e., circular), elliptical, triangular, square, rectangular, pentagonal, hexagonal, or any other polymeric shape. In some embodiments, the cross-section of the polymeric linker is the same shape as the cross-section of an agent-containing structural member attached to the polymeric linker. In some embodiments, the cross-section of the polymeric linker has a larger area than the cross-section of the agent-containing structural member, a smaller area than the cross-section of the agent-containing structural member, or approximately the same area as the cross-section of the attached agent-containing structural member.

In some embodiments, a polymeric linker may comprise a polylactic acid (PLA), a polycaprolactone (PCL), or another suitable polymer.

### Time-Dependent Disintegrating Matrices (Time-Dependent Linkers)

A time-dependent linker degrades in a predictable, time-dependent manner under aqueous conditions, such as when the gastric residence system is deployed in the stomach of an individual. The time-dependent polymeric linkers control the residence time of the gastric residence system in the stomach. The time-dependent polymeric linkers are designed to degrade, dissolve, mechanically weaken, or break gradually over time. After the desired residence period, the time-dependent polymeric linker has degraded, dissolved, disassociated, or mechanically weakened, or has broken, to the point where the gastric residence system can pass through the pyloric valve, exiting the gastric environment and entering the small intestine, for eventual elimination from the body.

The time-dependent polymeric linker preferably comprises a pH-independent degradable polymer, which degrades under aqueous conditions in a pH-independent or approximately pH-independent manner. Exemplary pH-independent degradable polymer include PLGA, PLA, PCL, polydioxanone, cellulose, or blends or copolymers thereof.

The time-dependent polymeric linker can include poly(lactic-co-glycolide) (PLGA).

In some embodiments, the PLGA of the time-dependent polymeric linker comprises copolymer of DL-lactide and glycolide (50/50 molar ratio) having a viscosity midpoint between about 0.32 dl/g to about 0.48 dl/g (such as about 0.4 dl/g) (such as the PLGA sold under the tradename Purasorb® PDLG 5004, available from Corbion). In some embodiments, the PLGA of the time-dependent polymeric linker comprises acid terminated copolymer of DL-lactide and glycolide (50/50 molar ratio) having a viscosity midpoint between about 0.32 dl/g to about 0.48 dl/g (such as about 0.4 dl/g) (such as the PLGA sold under the tradename Purasorb® PDLG 5004A available from Corbion). In some embodiments, the PLGA of the time-dependent polymeric linker comprises a mixture of (a) poly(D,L-lactic-co-glycolide) with a ratio of lactide monomers to glycolide monomers of about 50:50 (such as the PLGA sold under the tradename Purasorb® PDLG 5004, available from Corbion), and (b) acid-terminated poly(D,L-lactic-co-glycolide) with a ratio of lactide monomers to glycolide monomers of about 50:50 (such as the PLGA sold under the tradename Purasorb® PDLG 5004A, available from Corbion).

The one or more additional linker polymers included in the polymer linker may be homogenously mixed with the PLGA. In some embodiments, the one or more additional linker polymers are miscible with the PLGA. The one or more additional linker polymers may be a non-degradable polymer (that is, not degradable or in the gastric or enteric environment, or an aqueous solution of pH 1.6 (representing the gastric environment) or pH 6.5 (representing the enteric environment), and is optionally present in the time-dependent polymeric linker is an amount such that the time-dependent polymeric linker does not break during the gastric residence period.

Bonding of the polymeric linker to a directly adjacent member may be improved if at least one polymer is common to both the adjacent member and the time-dependent polymeric linker. In some embodiments, the at least one common polymer is polycaprolactone (PCL).

In some embodiments, the one or more additional linker polymers comprises a PCL. The time-dependent polymeric linker may be directly joined or bonded to another member of the gastric residence system (such as the structural member comprising the drug and the carrier polymer, a coupling member, the enteric polymeric linker, or a central structural member), which may also include a PCL, which may be the same PCL in the time-dependent polymeric linker or a different PCL as the one in the polymeric linker, and which may be at the same concentration or a different concentration. A different PCL in the time-dependent polymeric linker and the other member directly joined or bonded to the time-dependent linker may differ, for example, in the weight-average molecular weight of the PCL, the inherent viscosity of the PCL, or the proportions of PCL (for example, when a blend of two or more PCL polymers are used). In some embodiments, the time-dependent disintegrating matrix comprises about 40 wt % to about 50 wt % PCL. In some embodiments, the time-dependent disintegrating matrix comprises about 43 wt % to about 47 wt % PCL. In some embodiments, the time-dependent disintegrating matrix comprises about 45 wt % PCL. In some embodiments, the time-dependent disintegrating matrix comprises about 44.95 wt % PCL.

The time-dependent polymeric linker may further include one or more plasticizers, such as polyethylene glycol. The term “polyethylene glycol” is used interchangeably herein with the terms “polyethylene oxide” and “PEO.” In some embodiments, the molecular weight of the polyethylene glycol is about 90K to about 110K, such as 100 k (also referred to as 100K or 100 kDa. In some embodiments, the time-dependent disintegrating matrix comprises polyethylene glycol with molecular weight of about 100 k (polyethylene glycol 100 k). In some embodiments, the time-dependent disintegrating matrix comprises about 0.5 wt % to about 5 wt % polyethylene glycol 100 k. In some embodiments, the time-dependent disintegrating matrix comprises about 1 wt % to about 3 wt % polyethylene glycol 100 k. In some embodiments, the time-dependent disintegrating matrix comprises about 2 wt % polyethylene glycol 100 k.

In some embodiments, the time-dependent disintegrating matrix includes a color-absorbing dyes (also referred to as a colorant or a pigment). A color-absorbing dye may be included to enhance bonding or attachment of the polymeric linker to other gastric residence system components. Color-absorbing dyes can absorb heat during the laser-welding, infrared welding, or other heat-induced attachment, which increases the tensile strength of the resulting bond. Exemplary color-absorbing dyes include iron oxide and carbon black. The time-dependent disintegrating matrix may include the color-absorbing dye in an amount of up to about 5%, such as up to about 4%, up to about 3%, up to about 2%, up to about 1%, up to about 0.5%, up to about 0.3%, up to about 0.2%, up to about 0.1%, or up to about 0.05%. In some embodiments, the time-dependent disintegrating matrix comprises about 0.005 wt % to about 0.2 wt % color-absorbing dye. In some embodiments, the time-dependent disintegrating matrix comprises about 0.01 wt % to about 0.1 wt % color-absorbing dye. In some embodiments, the time-dependent disintegrating matrix comprises about 0.05 wt % color-absorbing dye. In some embodiments, the color-absorbing dye is E172.

In one example of a time-dependent disintegrating matrix, the time-dependent disintegrating matrix comprises about 40 wt % to about 50 wt % PCL, about 30 wt % to about 40 wt % of acid terminated copolymer of DL-lactide and glycolide (50/50 molar ratio) having a viscosity midpoint of about 0.4 dl/g, about 10 wt % to about 25 wt % of copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172.

In another example of a time-dependent disintegrating matrix, the time-dependent disintegrating matrix comprises about 43 wt % to about 47 wt % PCL, about 33 wt % to about 37 wt % of acid terminated copolymer of DL-lactide and glycolide (50/50 molar ratio) having a viscosity midpoint of about 0.4 dl/g, about 15 wt % to about 20 wt % of copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172.

In another example of a time-dependent disintegrating matrix, the time-dependent disintegrating matrix comprises about 44.95 wt % PCL, about 35 wt % of acid terminated copolymer of DL-lactide and glycolide (50/50 molar ratio) having a viscosity midpoint of about 0.4 dl/g, about 18 wt % of copolymer of DL-lactide and glycolide (50/50 molar ratio) having a viscosity midpoint of about 0.4 dl/g, about 2 wt % of polyethylene glycol 100 k and about 0.05 wt % color-absorbing dye E172.

In some embodiments, a dosage form for administration of one or more agents comprises a gastric residence system, wherein the gastric residence system comprises a time-dependent disintegrating matrix comprising about 44.95 wt % of polycaprolactone (PCL), such as PCL having a viscosity midpoint between about 1.5 dl/g to about 2.1 dl/g, such as Corbion PC17. In some embodiments, the gastric residence system comprises a time-dependent disintegrating matrix comprising about 35.0 wt % of an acid terminated copolymer of DL-lactide and glycolide (50/50 molar ratio) having a viscosity midpoint between about 0.32 dl/g to about 0.48 dl/g (such as about 0.4 dl/g), such as PDLG 5004A. In some embodiments, the gastric residence system comprises a time-dependent disintegrating matrix comprising about 18.0 wt % of a copolymer of DL-lactide and glycolide (50/50 molar ratio) having a viscosity midpoint between about 0.32 dl/g to about 0.48 dl/g (such as about 0.4 dl/g), such as PDLG 5004. In some embodiments, the gastric residence system comprises a time-dependent disintegrating matrix comprising about 2.0 wt % of polyethylene glycol, such as polyethylene glycol with average molecular weight of 100,000, such as PEO 100K. In some embodiments, the gastric residence system comprises a time-dependent disintegrating matrix comprising about 0.05 wt % of iron oxide, such as E172. In some embodiments, a dosage form for administration of one or more agents comprises a gastric residence system, wherein the gastric residence system comprises a time-dependent disintegrating matrix comprising about 44.95 wt % of Corbion PC17, about 35.0 wt % of PDLG 5004A, about 18.0 wt % of PDLG 5004, about 2.0 wt % of PEO 100K, and about 0.05 wt % of E172.

Exemplary amounts of the components for the time-dependent disintegrating matrix are provided in the table below. The amounts are given in approximate weight percent, with the understanding that when ranges are provided, the amounts are chosen so as to add up to 100%.

| Time-dependent | | | |
|---|---|---|---|
| disintegrating matrix | Formulation 1 | Formulation 2 | Formulation 3 |
| PCL | 40-50 | 43-47 | 44.95 |
| PDLG5004A | 30-40 | 33-37 | 35 |
| PDLG5004 | 10-25 | 15-20 | 18 |
| PEO(100k) | 0.5-5 | 1-3 | 2 |
| coloring (optional) | 0.005-0.2 | 0.01-0.1 | 0.05 (e.g., E172) |

### Enteric Disintegrating Matrices (Enteric Linkers)

The pH-dependent disintegrating matrices provide a safety mechanism for the gastric residence systems. If the system exits the stomach prematurely, that is, with all of the time-dependent disintegrating matrices intact, the pH-dependent disintegrating matrices will degrade, dissolve, disassociate, or mechanically weaken in the high pH environment of the small intestine, permitting the gastric residence system to pass readily through the small intestine. In addition, after passage of the gastric residence system once the time-dependent disintegrating matrices degrade, dissolve, disassociate, or mechanically weaken in the gastric environment, exposure of the pH-dependent disintegrating matrices to the high pH of the small intestine will provide further weakening and/or break-up of the system, for ready passage through the small intestine.

If the gastric residence system passes prematurely into the small intestine in an intact form, the system may be designed to break down much more rapidly to avoid intestinal obstruction. This is readily accomplished by using an enteric polymeric linker that includes an enteric polymer in addition to an additional linker polymer (such as a carrier polymer), which weakens or degrades within the intestinal environment. Enteric polymers are relatively resistant to the acidic pH levels encountered in the stomach, but dissolve rapidly at the higher PH levels found in the duodenum. Use of enteric polymeric linkers as safety elements protects against undesired passage of the intact gastric residence system into the small intestine. The use of enteric polymeric linker also provides a manner of removing the gastric residence system prior to its designed residence time; should the system need to be removed, the patient can drink a mildly alkaline solution, such as a sodium bicarbonate solution, or take an antacid preparation such as hydrated magnesium hydroxide (milk of magnesia) or calcium carbonate, which will raise the pH level in the stomach and cause rapid degradation of the enteric polymeric linker.

Weakening or degradation of the enteric polymeric linker may be measured in references to a loss of the flexural modulus or breakage of the polymeric linker under a given condition (e.g., enteric conditions or gastric conditions). The enteric linkers weaken, degrade, or break in the intestinal environment relatively quickly, while retain much of their flexural modulus in the gastric environment. Stomach conditions may be simulated using an aqueous solution, such FaSSGF, at a pH of 1.6 and at 37° ° C., and intestinal conditions may be simulated using an aqueous solution, such as FaSSIF, at a pH 6.5 at 37° ° C.

In some embodiments, the enteric disintegrating matrix comprises hydroxypropyl methylcellulose acetate succinate (HPMCAS). For example, in some embodiments, the enteric disintegrating matrix includes about 60 wt % to about 70 wt % HPMCAS. In some embodiments, the enteric disintegrating matrix includes about 62 wt % to about 66 wt % HPMCAS. In some embodiments, the enteric disintegrating matrix includes about 63.95 wt % HPMCAS.

The enteric polymer is combined with one or more additional polymers (such as one or more carrier polymers) in the enteric linker, preferably in a homogenous mixture. For example, the enteric polymer and the additional linker polymer may be homogenously blended together before the mixture is extruded, and the extruded material being cut to a desired size for the polymeric linker. In some embodiments, the one or more additional linker polymers are miscible with the enteric polymer. The one or more additional linker polymers may be a non-degradable polymer (that is, not degradable or in the gastric or enteric environment, or an aqueous solution of pH 1.6 (representing the gastric environment) or pH 6.5 (representing the enteric environment).

Bonding of the polymeric linker to a directly adjacent member may be improved if at least one polymer is common to both the adjacent member and the enteric polymeric linker. That is, one of the one or more additional linker polymers in the enteric linker may be the same (or the same polymer type) as at least one polymer in a directly adjacent component (or, optionally, both directly adjacent components) of the gastric residence system. For example, if the enteric polymeric linker is bonded directly to a structural member comprising a carrier polymer, in some embodiments the one or more additional linker polymers also includes the carrier polymer (in addition to the PLGA in the time-dependent polymeric linker) at the same or different concentration. Exemplary carrier polymers include, but are not limited to, polylactic acid (PLA), and polycaprolactone (PCL), among others described herein.

In some embodiments, the one or more additional linker polymers in the enteric linker comprises a PCL. The enteric polymeric linker may be directly joined or bonded to another member of the gastric residence system (such as the structural member comprising the drug and the carrier polymer, a coupling member, the time-dependent polymeric linker, or a central structural member), which may also include a PCL, which may be the same PCL in the enteric polymeric linker or a different PCL as the one in the enteric polymeric linker, and which may be at the same concentration or a different concentration. A different PCL in the enteric polymeric linker and the other member directly joined or bonded to the enteric linker may differ, for example, in the weight-average molecular weight of the PCL, the inherent viscosity of the PCL, or the proportions of PCL (for example, when a blend of two or more PCL polymers are used). In some embodiments, the enteric disintegrating matrix comprises about 30 wt % to about 40 wt % PCL. In some embodiments, the enteric disintegrating matrix comprises about 32 wt % to about 37 wt % PCL. In some embodiments, the enteric disintegrating matrix comprises about 34 wt % PCL. In some embodiments, the enteric disintegrating matrix comprises about 33.95 wt % PCL.

The enteric disintegrating matrix may further include one or more plasticizers, such as a poloxamer (e.g., Poloxamer 407, or “P407”). In some embodiments, the enteric disintegrating matrix comprises about 0.5 wt % to about 5 wt % poloxamer. In some embodiments, the enteric disintegrating matrix comprises about 1 wt % to about 3 wt % poloxamer. In some embodiments, the enteric disintegrating matrix comprises about 2 wt % poloxamer.

In some embodiments, the enteric disintegrating matrix includes a color-absorbing dyes (also referred to as a colorant or a pigment). A color-absorbing dye may be included to enhance bonding or attachment of the polymeric linker to other gastric residence system components. Color-absorbing dyes can absorb heat during the laser-welding, infrared welding, or other heat-induced attachment, which increases the tensile strength of the resulting bond. Exemplary color-absorbing dyes include iron oxide and carbon black. The enteric polymeric linker may include the color-absorbing dye in an amount of up to about 5%, such as up to about 4%, up to about 3%, up to about 2%, up to about 1%, up to about 0.5%, up to about 0.3%, up to about 0.2%, or up to about 0.1%. In some embodiments, the enteric disintegrating matrix comprises about 0.01 wt % to about 0.2 wt % color-absorbing dye ferrosoferric oxide. In some embodiments, the enteric disintegrating matrix comprises about 0.05 wt % to about 0.15 wt % color-absorbing dye ferrosoferric oxide. In some embodiments, the enteric disintegrating matrix comprises about 0.1 wt % color-absorbing dye ferrosoferric oxide.

In some embodiments, the enteric disintegrating matrix comprises about 59 wt % to about 69 wt % HPMCAS, about 29 wt % to about 39 wt % PCL, and about 0.5 wt % to about 5 wt % poloxamer (such as P407). Optionally, the enteric disintegrating matrix further comprises iron oxide, for example about 0.01 wt % to about 0.2 wt % iron oxide (such as E172).

In some embodiments, the enteric disintegrating matrix comprises about 62 wt % to about 66 wt % HPMCAS, about 32 wt % to about 36 wt % PCL, and about 1 wt % to about 3 wt % poloxamer (such as P407). Optionally, the enteric disintegrating matrix further comprises iron oxide, for example about 0.05 wt % to about 0.15 wt % iron oxide (such as E172).

In some embodiments, the enteric disintegrating matrix comprises about 63.95 wt % HPMCAS, about 33.95 wt % PCL, and about 2 wt % poloxamer (such as P407). Optionally, the enteric disintegrating matrix further comprises iron oxide, for example about 0.1 wt % iron oxide (such as E172).

In some embodiments, a dosage form for administration of one or more agents comprises a gastric residence system, wherein the gastric residence system comprises a pH-dependent disintegrating matrix comprising about 33.95 wt % of polycaprolactone (PCL), such as PCL having a viscosity midpoint between about 1.5 dl/g to about 2.1 dl/g, such as Corbion PC17. In some embodiments, the gastric residence system comprises a pH-dependent disintegrating matrix comprising about 63.95 wt % of hypromellose acetate succinate, such as HPMCAS-MG. In some embodiments, the gastric residence system comprises a pH-dependent disintegrating matrix comprising about 2.0 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407 (P407, a poly(ethylene glycol)-block-poly(propylene glycol)-block-poly(ethylene glycol) polymer with a polyoxypropylene molecular mass of about 4000 and about 70% polyoxyethylene content). In some embodiments, the gastric residence system comprises a pH-dependent disintegrating matrix comprising about 0.1 wt % of iron oxide, such as E172. In some embodiments, a dosage form for administration of one or more agents comprises a gastric residence system, wherein the gastric residence system comprises a pH-dependent disintegrating matrix comprising about 33.95 wt % of Corbion PC17, about 63.95 wt % of HPMCAS-MG, about 2.0 wt % of P407, and about 0.1 wt % of E172.

Exemplary amounts of the components for the enteric disintegrating matrix are provided in the table below. The amounts are given in approximate weight percent, with the understanding that when ranges are provided, the amounts are chosen so as to add up to 100%.

| Enteric | | | |
|---|---|---|---|
| disintegrating matrix | Formulation 1 | Formulation 2 | Formulation 3 |
| PCL | 29-39 | 32-36 | 33.95 |
| HPMCAS | 59-69 | 62-66 | 63.95 |
| P407 | 0.5-5 | 1-3 | 2 |
| coloring (optional) | 0.01-0.2 | 0.05-0.15 | 0.1 (e.g. E172) |

### Arm Tip Disintegrating Matrices

In some embodiments, the gastric residence system comprises arms comprising a third disintegrating matrix in addition to the time-dependent disintegrating matrix and the enteric disintegrating matrix. In some embodiments, the third disintegrating matrix is a filament holding segment (i.e., segment to which the filament is attached). In some embodiments, the third disintegrating is the distal segment of the residence system arm, i.e., the tip of the arm.

In some embodiments, the third disintegrating matrix comprises hydroxypropyl methylcellulose acetate succinate (HPMCAS). For example, in some embodiments the third disintegrating matrix includes about 60 wt % to about 70 wt % HPMCAS. In some embodiments, the third disintegrating matrix includes about 63 wt % to about 67 wt % HPMCAS. In some embodiments, the third disintegrating matrix includes about 64.9 wt % HPMCAS.

In some embodiments, the third disintegrating matrix comprises a polymer common with one or other segment in the gastric residence system arm. In some embodiments, the third disintegrating matrix comprises polycaprolactone (PCL). In some embodiments, the third disintegrating matrix comprises about 25 wt % to about 35 wt % PCL. In some embodiments, the third disintegrating matrix comprises about 28 wt % to about 32 wt % PCL. In some embodiments, the third disintegrating matrix comprises about 30 wt % PCL.

In some embodiments, the third disintegrating matrix comprises one or more acids, such as stearic acid. In some embodiments, the third disintegrating matrix comprises about 1 wt % to about 5 wt % stearic acid. In some embodiments, the third disintegrating matrix comprises about 2 wt % to about 3 wt % stearic acid. In some embodiments, the third disintegrating matrix comprises about 2.5 wt % stearic acid.

In some embodiments, the third disintegrating matrix may further include one or more plasticizers, such as a propylene glycol. In some embodiments, the third disintegrating matrix comprises about 1 wt % to about 5 wt % propylene glycol. In some embodiments, the third disintegrating matrix comprises about 2 wt % to about 3 wt % propylene glycol. In some embodiments, the third disintegrating matrix comprises about 2.5 wt % propylene glycol.

In some embodiments, the third disintegrating matrix includes a color-absorbing dyes (also referred to as a colorant or a pigment). A color-absorbing dye may be included to enhance bonding or attachment of the polymeric linker to other gastric residence system components. Color-absorbing dyes can absorb heat during the laser-welding, infrared welding, or other heat-induced attachment, which increases the tensile strength of the resulting bond. Exemplary color-absorbing dyes include iron oxide and carbon black. The third disintegrating matrix may include the color-absorbing dye in an amount of up to about 5%, such as up to about 4%, up to about 3%, up to about 2%, up to about 1%, up to about 0.5%, up to about 0.3%, up to about 0.2%, or up to about 0.1%. In some embodiments, the third disintegrating matrix comprises about 0.01 wt % to about 0.5 wt % color-absorbing dye. In some embodiments, the third disintegrating matrix comprises about 0.05 wt % to about 0.15 wt % color-absorbing dye. In some embodiments, the third disintegrating matrix comprises about 0.1 wt % color-absorbing dye. In some embodiments, the third disintegrating matrix comprises about 0.025% ferrosoferric oxide and about 0.075% FD&C Red 40. In some embodiments, the third disintegrating matrix comprises about 0.025% ferrosoferric oxide and about 0.075% FD&C Red 40.

In some embodiments, the third disintegrating matrix comprises about 60 wt % to about 70 wt % HPMCAS, about 25 wt % to about 35 wt % PCL, about 1 wt % to about 5 wt % propylene glycol and about 1 wt % to about 5 wt % stearic acid. Optionally, the third disintegrating matrix further comprises about 0.01 wt % to about 0.5 wt % iron oxide.

In some embodiments, the third disintegrating matrix comprises about 63 wt % to about 67 wt % HPMCAS, about 28 wt % to about 32 wt % PCL, about 2 wt % to about 3 wt % propylene glycol and about 2 wt % to about 3 wt % stearic acid. Optionally, the third disintegrating matrix further comprises about 0.05 wt % to about 0.15 wt % iron oxide.

In some embodiments, the third disintegrating matrix comprises 64.9 wt % HPMCAS, about 30 wt % PCL, about 2.5 wt % propylene glycol and about 2.5 wt % stearic acid. Optionally, the third disintegrating matrix further comprises about 0.1 wt % iron oxide, for example about 0.025% ferrosoferric oxide and about 0.075% FD&C Red 40.

Exemplary amounts of the components for the third disintegrating matrix are provided in the table below. The amounts are given in approximate weight percent, with the understanding that when ranges are provided, the amounts are chosen so as to add up to 100%.

| ODMTEP | | | |
|---|---|---|---|
| disintegrating | | | |
| matrix | Formulation 1 | Formulation 2 | Formulation 3 |
| PCL | 25-35 | 28-32 | 30 |
| HPMCAS | 60-70 | 63-67 | 64.9 |
| Stearic acid | 1-5 | 2-3 | 2.5 |
| Propylene Glycol | 1-5 | 2-3 | 2.5 |
| Iron oxide | 0.01-0.5 | 0.05-0.15 | 0.1 (e.g. 0.025% |
| | | | ferrosoferric oxide |
| | | | and 0.075% FD&C |
| | | | Red 40) |

### Inert Segments

In some embodiments, the gastric residence system comprises one or more inert segments. In some embodiments, the inert segment comprises one or more radiopaque substances.

In some embodiments, the inert segment comprises a common polymer with other segments in the gastric residence system. In some embodiments, the inert segment comprises polycaprolactone (PCL). In some embodiments, the inert segment comprises about 61 wt % to about 71 wt % PCL. In some embodiments, the inert segment comprises about 64 wt % to about 69 wt % PCL. In some embodiments, the inert segment comprises about 66.5 wt % PCL. In some embodiments, the inert segment comprises about 66.45 wt % PCL.

In some embodiments, the inert segment comprises vinylpyrrolidone-vinyl acetate copolymer in a ratio of 6:4 by mass (i.e. copovidone, such as Kollidon VA64). In some embodiments, the inert segment comprises about 27 wt % to about 37 wt % copovidone. In some embodiments, the inert segment comprises about 30 wt % to about 34 wt % copovidone. In some embodiments, the inert segment comprises about 32 wt % copovidone.

The inert segment may further include one or more plasticizers, such as a poloxamer (e.g., Poloxamer 407, or “P407”). In some embodiments, the inert segment comprises about 0.2 wt % to about 4 wt % poloxamer. In some embodiments, the inert segment comprises about 0.5 wt % to about 2.5 wt % poloxamer. In some embodiments, the inert segment comprises about 1.5 wt % poloxamer.

In some embodiments, the inert segment includes a color-absorbing dyes (also referred to as a colorant or a pigment). The inert segment may include the color-absorbing dye in an amount of up to about 5%, such as up to about 4%, up to about 3%, up to about 2%, up to about 1%, up to about 0.5%, up to about 0.3%, up to about 0.2%, up to about 0.1%, or up to 0.05%. In some embodiments, the inert segment comprises about 0.005 wt % to about 0.2 wt % color-absorbing dye. In some embodiments, the inert segment comprises about 0.01 wt % to about 0.1 wt % color-absorbing dye. In some embodiments, the inert segment comprises about 0.05 wt % color-absorbing dye. In some embodiments, the color-absorbing dye is FD&C Blue #1 Alum Lake.

In some embodiments, the inert segment comprises about 61 wt % to about 71 wt % PCL, about 27 wt % to about 37 wt % copovidone, about 0.2 wt % to about 4 wt % poloxamer. Optionally, the inert segment further comprises color-absorbing dye, for example about 0.005 wt % to about 0.2 wt % color-absorbing dye FD&C Blue #1 Alum Lake.

In some embodiments, the inert segment comprises about 64 wt % to about 69 wt % PCL, about 30 wt % to about 34 wt % copovidone, about 0.5 wt % to about 2.5 wt % poloxamer. Optionally, the inert segment further comprises color-absorbing dye, for example about 0.01 wt % to about 0.1 wt % color-absorbing dye FD&C Blue #1 Alum Lake.

In some embodiments, the inert segment comprises about 66.45 wt % PCL, about 32 wt % copovidone, about 1.5 wt % poloxamer. Optionally, the inert segment further comprises color-absorbing dye, for example about 0.05 wt % color-absorbing dye FD&C Blue #1 Alum Lake.

Exemplary amounts of the components for one embodiment of the inert segment (e.g. inactive spacer) are provided in the table below. The amounts are given in approximate weight percent, with the understanding that when ranges are provided, the amounts are chosen so as to add up to 100%.

| Inert Segment | Formulation | Formulation | Formulation |
|---|---|---|---|
| (Inactive spacer) | 1 | 2 | 3 |
| PCL | 61-71 | 64-69 | 66.45 |
| VA64 | 27-37 | 30-34 | 32 |
| P407 | 0.2-4 | 0.5-2.5 | 1.5 |
| coloring (optional) | 0.005-0.2 | 0.01-0.1 | 0.05 (e.g. |
| | | | Blue #1) |

In some embodiments, the gastric residence system comprises one or more inert segments, wherein the inert segment comprises one or more radiopaque substances. In some embodiments, the gastric residence system comprises an inert segment, wherein the inert segment is a radiopaque segment.

In some embodiments, the inert segment comprises a common polymer with other segments in the gastric residence system. In some embodiments, the inert segment comprises polycaprolactone (PCL). In some embodiments, the inert segment comprises about 65 wt % to about 75 wt % PCL. In some embodiments, the inert segment comprises about 68 wt % to about 72 wt % PCL. In some embodiments, the inert segment comprises about 70 wt % PCL.

In some embodiments, the inert segment comprises a radiopaque substance. In some embodiments, the inert segment comprises a radiopaque substance, wherein the radiopaque substance is (BiO)₂CO₃. In some embodiments, the inert segment comprises (BiO)₂CO₃. In some embodiments, the inert segment comprises about 25 wt % to about 35 wt % (BiO)₂CO₃. In some embodiments, the inert segment comprises about 28 wt % to about 32 wt % (BiO)₂CO₃. In some embodiments, the inert segment comprises about 30 wt % (BiO)₂CO₃.

In some embodiments, the inert segment comprises about 65 wt % to about 75 wt % PCL, and about 25 wt % to about 35 wt % (BiO)₂CO₃. In some embodiments, the inert segment comprises about 68 wt % to about 72 wt % PCL, and about 28 wt % to about 32 wt % (BiO)₂CO₃. In some embodiments, the inert segment comprises about 70 wt % PCL, and about 30 wt % (BiO)₂CO₃.

Exemplary amounts of the components for one embodiment of the inert segment (e.g. rPCL segment) are provided in the table below. The amounts are given in approximate weight percent, with the understanding that when ranges are provided, the amounts are chosen so as to add up to 100%.

| Inert segment- | | | |
|---|---|---|---|
| rPCL (radiopaque) | Formulation 1 | Formulation 2 | Formulation 3 |
| PCL | 65-75 | 68-72 | 70 |
| (BiO)₂CO₃ | 25-35 | 28-32 | 30 |

### Carrier Polymer-Agent Segments (Drug-Eluting Segments) Comprising a Combination Buprenorphine-Naloxone Formulation

The carrier polymer-agent segments, or drug-eluting segments, release an agent in a controlled manner during the period that the gastric residence system resides in the stomach. The carrier polymer may be blended with the agent, and formed into segments which are then assembled with the other components described herein to manufacture the gastric residence system. The composition of such carrier polymer-agent blends is provided below for specific drug formulations, including formulations comprising a combination of buprenorphine and naloxone.

Both actives, buprenorphine and naloxone, are formulated into a single segment or component of a gastric residence system—a drug-eluting segment. This is designed such that a user cannot easily manipulate the gastric residence system to remove the naloxone such that they can consume the buprenorphine only. Alternatively, the gastric residence system could be designed such that the buprenorphine and naloxone are provided in separate components of the gastric residence system. However, this would allow for easier manipulation, since a user could physically separate the two components (i.e., the buprenorphine component and the naloxone component) to consume the buprenorphine component only. Accordingly, drug-eluting segments or components described comprise both buprenorphine and naloxone.

In some embodiments, the buprenorphine and naloxone of the drug-eluting segments may release at similar rates. In some embodiments, the buprenorphine and naloxone of the drug-eluting segments may release at different rates. For example, the naloxone may release from the drug-eluting segments faster than the buprenorphine. In either case, the fact that the buprenorphine and naloxone are formulated into a single component of the gastric residence system (i.e., a drug-eluting segment) greatly minimizes a user from separating the naloxone from the buprenorphine. For example, a user might try to elute the naloxone from the gastric residence system outside of the body and consume the remaining buprenorphine. However, for gastric residence systems in which the buprenorphine and naloxone have similar release profiles, very little if any buprenorphine, if any, will remain once the naloxone has completely eluted off. For gastric residence systems in which the naloxone releases faster than the buprenorphine, a significant amount of the buprenorphine will elute off in the time it takes the naloxone to completely elute from the gastric residence system. Thus, a user will be left with a significantly smaller amount of buprenorphine than that which was originally present in the gastric residence system. In either case, abuse of the gastric residence system is minimized.

In some embodiments, a dosage form for administration of buprenorphine and naloxone comprises a gastric residence system comprising about 10 mg to about 150 mg of buprenorphine. In some embodiments, a dosage form for administration of buprenorphine and naloxone comprises a gastric residence system comprising about 40 mg to about 115 mg of buprenorphine. In some embodiments, a dosage form for administration of buprenorphine and naloxone comprises a gastric residence system comprising less than or equal to about 150, about 140, about 130, about 120, about 110, about 100, about 90, about 80, about 70, about 60, about 50, about 40, about 30, or about 20 mg of buprenorphine. In some embodiments, a dosage form for administration of buprenorphine and naloxone comprises a gastric residence system comprising greater than or equal to about 10, about 20, about 30, about 40, about 50, about 60, about 70, about 80, about 90, about 100, about 110, about 120, about 130, or about 140 mg buprenorphine.

In some embodiments, a dosage form for administration of buprenorphine and naloxone comprises a gastric residence system comprising about 1 mg to about 40 mg naloxone. In some embodiments, a dosage form for administration of buprenorphine and naloxone comprises a gastric residence system comprising about 3.5 mg to about 10 mg naloxone. In some embodiments, a dosage form for administration of buprenorphine and naloxone comprises a gastric residence system comprising less than or equal to about 40, about 35, about 30, about 25, about 20, about 15, about 10, or about 5 mg naloxone. In some embodiments, a dosage form for administration of buprenorphine and naloxone comprises a gastric residence system comprising greater than or equal to about 1, about 5, about 10, about 15, about 20, about 25, about 30, or about 35 mg naloxone.

In some embodiments, the dosage form comprises a gastric residence system, wherein the gastric residence system comprises one or more drug-eluting segments, each of the one or more drug-eluting segments comprising about 10 mg to about 30 mg buprenorphine and about 1 mg to about 5 mg naloxone. In some embodiments, each drug-eluting segment comprises less than or equal to about 30, 25, 20, or 15 mg buprenorphine. In some embodiments, each drug-eluting segment comprises more than or equal to about 10, 15, 20, or 25 mg naloxone.

In some embodiments, a buprenorphine-naloxone combination formulation comprises about 30 wt % to about 55 wt % of buprenorphine. In some embodiments, a buprenorphine-naloxone combination formulation comprises less than or equal to about 55, about 50, about 45, about 40, or about 35 wt % buprenorphine. In some embodiments, a buprenorphine-naloxone combination formulation comprises greater than or equal to about 30, about 35, about 40, about 45, or about 50 wt % buprenorphine.

In some embodiments, a buprenorphine-naloxone combination formulation comprises about 0.5 wt % to about 10 wt % of naloxone. In some embodiments, a buprenorphine-naloxone combination formulation comprises less than or equal to about 10, about 7.5, about 5, about 2.5, or about 1 wt % naloxone. In some embodiments, a buprenorphine-naloxone combination formulation comprises greater than or equal to about 0.5, about 1, about 2.5, about 5, or about 7.5 wt % naloxone.

In some embodiments, the combination buprenorphine and naloxone formulations may include a ratio of naloxone (NAL) to buprenorphine (BUP) of between about 1:1 and about 1:20 NAL:BUP. In some embodiments, the ratio may be about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, or about 1:20 NAL:BUP. In some embodiments, the amount of buprenorphine in a combination buprenorphine and naloxone formulation may be less than or equal to about 20, about 18, about 15, about 12, about 10, about 8, about 5, about 4, about 3, or about 2 times the amount of naloxone by weight. In some embodiments, the amount of buprenorphine in a combination buprenorphine and naloxone formulation may be greater than or equal to about 1, about 2, about 3, about 4, about 5, about 8, about 10, about 12, about 15, or about 18 times the amount of naloxone by weight. In some embodiments, the ratio of NAL:BUP may depend on the release properties of each active ingredient.

Buprenorphine-naloxone combination formulations comprise about 30 wt % to 50 wt % polycaprolactone (PCL), such as PCL having a viscosity midpoint between about 1.5 dl/g to about 2.1 dl/g, such as Corbion PC17. In some embodiments, a buprenorphine-naloxone combination formulation comprises less than or equal to about 50, about 45, about 40, or about 35 wt % PCL. In some embodiments, a buprenorphine-naloxone combination formulation comprises greater than or equal to about 30, about 35, about 40, or about 45 wt % PCL.

In some embodiments, a buprenorphine-naloxone combination formulation comprises about 1 wt % to about 15 wt % polyethylene oxide (PEO) (e.g., PEO_{100K}). In some embodiments, a buprenorphine-naloxone combination formulation comprises less than or equal to about 15, about 12.5, about 10, about 7.5, about 5, or about 2.5 wt % PEO. In some embodiments, a buprenorphine-naloxone combination formulation comprises greater than or equal to about 1, about 2.5, about 5, about 7.5, about 10, about 12.5 wt % PEO.

In some embodiments, a buprenorphine-naloxone combination formulation comprises about 1 wt % to about 5 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407. In some embodiments, a buprenorphine-naloxone combination formulation comprises less than or equal to about 5, about 4, about 3, or about 2 wt % poly(ethylene glycol)-block-poly(propylene glycol)-block-poly(ethylene glycol) polymers such as Poloxamer 407. In some embodiments, a buprenorphine-naloxone combination formulation comprises greater than or equal to about 1, about 2, about 3, or about 4 wt % poly(ethylene glycol)-block-poly(propylene glycol)-block-poly(ethylene glycol) polymers such as Poloxamer 407.

In some embodiments, a buprenorphine-naloxone combination formulation comprises about 0.1 wt % to about 5 wt % of Vitamin E succinate. In some embodiments, a buprenorphine-naloxone combination formulation may include less than or equal to about 5, about 4, about 3, about 2, about 1, about 0.5, or about 0.1 wt. % Vitamin E succinate. In some embodiments, a buprenorphine-naloxone combination formulation may include more than or equal to about 0.01, about 0.1, about 0.5, about 1, about 2, about 3, or about 4 wt. % Vitamin E succinate.

In some embodiments, a buprenorphine-naloxone combination formulation comprises about 0.1 wt % to about 1 wt % of colloidal silicon dioxide (SiO₂).

In some embodiments, wherein a buprenorphine-naloxone combination formulation comprises about 35 wt % to about 50 wt % of buprenorphine, the buprenorphine-naloxone combination formulation comprises about 1 to about 7 wt % naloxone, about 35 wt % to about 58 wt % of polycaprolactone (PCL), such as PCL having a viscosity midpoint between about 1.5 dl/g to about 2.1 dl/g, such as Corbion PC17. In some embodiments, a buprenorphine-naloxone combination formulation comprises about 1 wt % to about 15 wt % of PEO, such as PEO_{100K}. In some embodiments, a buprenorphine-naloxone combination formulation comprises about 2 wt % to about 4 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407. In some embodiments, a buprenorphine-naloxone combination formulation comprises about 0.2 wt % to about 0.8 wt % of Vitamin E succinate. In some embodiments, a buprenorphine-naloxone combination formulation comprises about 0.2 wt % to about 0.8 wt % of colloidal silicon dioxide (SiO₂).

In some embodiments, wherein a buprenorphine-naloxone combination formulation comprises about 42 wt % of buprenorphine, the buprenorphine-naloxone combination formulation comprises about 3.7 wt % naloxone, 43.3 wt % of polycaprolactone (PCL), such as PCL having a viscosity midpoint between about 1.5 dl/g to about 2.1 dl/g, such as Corbion PC17. In some embodiments, a buprenorphine-naloxone combination formulation comprises about 7 wt % of PEO, such as PEO_{100K}. In some embodiments, a buprenorphine-naloxone combination formulation comprises about 3.0 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407. In some embodiments, a buprenorphine-naloxone combination formulation comprises about 0.5 wt % of Vitamin E succinate. In some embodiments, a buprenorphine-naloxone combination formulation comprises about 0.5 wt % of colloidal silicon dioxide (SiO₂).

In some embodiments, a drug-eluting segment comprising a buprenorphine-naloxone combination formulation may be formed by extruding the buprenorphine-naloxone combination formulation. In some embodiments, the buprenorphine-naloxone combination formulation may be extruded to form a drug-eluting segment or component comprising a circular, triangular, or square-shaped cross-section.

Exemplary amounts of the components for some embodiments of a buprenorphine-naloxone combination formulation are provided in the table below. In some embodiments, a buprenorphine-naloxone combination formulation is used for the drug-eluting segment (and no additional formulations), these amounts correspond to the amounts of the drug-eluting segment. The amounts are given in approximate weight percent, with the understanding that when ranges are provided, the amounts are chosen so as to add up to 100%. “Pharm. accept. salt” indicates pharmaceutically acceptable salt thereof.

| Carrier polymer-arm | | | |
|---|---|---|---|
| segment | Formulation 1 | Formulation 2 | Formulation 3 |
| Buprenorphine (or | 35-55 | 40-50 | 45 |
| pharm. accept. salt) | | | |
| Naloxone (or | 1-8 | 3-6 | 4.5 |
| pharm. accept. salt) | | | |
| PCL | 30-50 | 35-45 | 39.5 |
| PEO_{100K} | 1-15 | 5-10 | 7.0 |
| P407 | 1-5 | 2-4 | 3.0 |
| Vit E | 0.1-1 | 0.2-0.8 | 0.5 |
| SiO₂ | 0.1-1 | 0.2-0.8 | 0.5 |

In some embodiments, a drug-eluting segment comprising a buprenorphine-naloxone combination formulation may release between about 5 and about 90% of the total buprenorphine content of the drug-eluting segment within the first 24 hours of residence. In some embodiments, a drug-eluting segment comprising a buprenorphine-naloxone combination formulation may release greater than or equal to about 5, 10, 15, 20, 25, 30, 35, 40, 45, 50, 55, 60, 65, 70, 75, 80, or 85% of the total buprenorphine content of the drug-eluting segment within the first 24 hours of residence. In some embodiments, a drug-eluting segment comprising a buprenorphine-naloxone combination formulation may release less than or equal to about 90, 85, 80, 75, 70, 65, 60, 55, 50, 45, 40, 35, 30, 25, 20, 15, or 10% of the total buprenorphine content of the drug-eluting segment within the first 24 hours of residence. In some embodiments, a drug-eluting segment comprising a buprenorphine-naloxone combination formulation may release between about 5 and about 90% of the total buprenorphine content of the drug-eluting segment after the first 24 hours of residence. In some embodiments, a drug-eluting segment comprising a buprenorphine-naloxone combination formulation may release greater than or equal to about 5, 10, 15, 20, 25, 30, 35, 40, 45, 50, 55, 60, 65, 70, 75, 80, or 85% of the total buprenorphine content of the drug-eluting segment after the first 24 hours of residence. In some embodiments, a drug-eluting segment comprising a buprenorphine-naloxone combination formulation may release less than or equal to about 90, 85, 80, 75, 70, 65, 60, 55, 50, 45, 40, 35, 30, 25, 20, 15, or 10% of the total buprenorphine content of the drug-eluting segment after the first 24 hours of residence. In some embodiments, a drug-eluting segment comprising a buprenorphine-naloxone combination formulation may release between about 5 and about 70% of the total buprenorphine content of the drug-eluting segment after the first 48 hours of residence. In some embodiments, a drug-eluting segment comprising a buprenorphine-naloxone combination formulation may release greater than or equal to about 5, 10, 15, 20, 25, 30, 35, 40, 45, 50, 55, 60, or 65% of the total buprenorphine content of the drug-eluting segment after the first 48 hours of residence. In some embodiments, a drug-eluting segment comprising a buprenorphine-naloxone combination formulation may release less than or equal to about 70, 65, 60, 55, 50, 45, 40, 35, 30, 25, 20, 15, or 10% of the total buprenorphine content of the drug-eluting segment after the first 48 hours of residence. In some embodiments, a drug-eluting segment comprising a buprenorphine-naloxone combination formulation may release between about 5 and about 50% of the total buprenorphine content of the drug-eluting segment after the first 72 hours of residence. In some embodiments, a drug-eluting segment comprising a buprenorphine-naloxone combination formulation may release greater than or equal to about 5, 10, 15, 20, 25, 30, 35, 40, or 45% of the total buprenorphine content of the drug-eluting segment after the first 72 hours of residence. In some embodiments, a drug-eluting segment comprising a buprenorphine-naloxone combination formulation may release less than or equal to about 50, 45, 40, 35, 30, 25, 20, 15, or 10% of the total buprenorphine content of the drug-eluting segment after the first 72 hours of residence. In some embodiments, a drug-eluting segment comprising a buprenorphine-naloxone combination formulation may release between about 5 and about 30% of the total buprenorphine content of the drug-eluting segment after the first 96 hours of residence. In some embodiments, a drug-eluting segment comprising a buprenorphine-naloxone combination formulation may release greater than or equal to about 5, 10, 15, 20, or 25% of the total buprenorphine content of the drug-eluting segment after the first 96 hours of residence. In some embodiments, a drug-eluting segment comprising a buprenorphine-naloxone combination formulation may release less than or equal to about 30, 25, 20, 15, or 10% of the total buprenorphine content of the drug-eluting segment after the first 96 hours of residence. In some embodiments, a drug-eluting segment comprising a buprenorphine-naloxone combination formulation may release between about 5 and about 20% of the total buprenorphine content of the drug-eluting segment after the first 120 hours of residence. In some embodiments, a drug-eluting segment comprising a buprenorphine-naloxone combination formulation may release greater than or equal to about 5, 10, or 15% of the total buprenorphine content of the drug-eluting segment after the first 120 hours of residence. In some embodiments, a drug-eluting segment comprising a buprenorphine-naloxone combination formulation may release less than or equal to about 20, 15, or 10% of the total buprenorphine content of the drug-eluting segment after the first 120 hours of residence.

In some embodiments, a drug-eluting segment comprising a buprenorphine-naloxone combination formulation may release between about 5 and about 100% of the total naloxone content of the drug-eluting segment within the first 24 hours of residence. In some embodiments, a drug-eluting segment comprising a buprenorphine-naloxone combination formulation may release greater than or equal to about 5, 10, 15, 20, 25, 30, 35, 40, 45, 50, 55, 60, 65, 70, 75, 80, 85, 90, or 95% of the total naloxone content of the drug-eluting segment within the first 24 hours of residence. In some embodiments, a drug-eluting segment comprising a buprenorphine-naloxone combination formulation may release less than or equal to about 100, 95, 90, 85, 80, 75, 70, 65, 60, 55, 50, 45, 40, 35, 30, 25, 20, 15, or 10% of the total naloxone content of the drug-eluting segment within the first 24 hours of residence. In some embodiments, a drug-eluting segment comprising a buprenorphine-naloxone combination formulation may release between about 0 and about 50% of the total naloxone content of the drug-eluting segment after the first 24 hours of residence. In some embodiments, a drug-eluting segment comprising a buprenorphine-naloxone combination formulation may release greater than or equal to about 0, 5, 10, 15, 20, 25, 30, 35, 40, or 45% of the total naloxone content of the drug-eluting segment after the first 24 hours of residence. In some embodiments, a drug-eluting segment comprising a buprenorphine-naloxone combination formulation may release less than or equal to about 50, 45, 40, 35, 30, 25, 20, 15, 10, or 5% of the total naloxone content of the drug-eluting segment after the first 24 hours of residence. In some embodiments, a drug-eluting segment comprising a buprenorphine-naloxone combination formulation may release between about 0 and about 50% of the total naloxone content of the drug-eluting segment after the first 48 hours of residence. In some embodiments, a drug-eluting segment comprising a buprenorphine-naloxone combination formulation may release greater than or equal to about 0, 5, 10, 15, 20, 25, 30, 35, 40, or 45% of the total naloxone content of the drug-eluting segment after the first 48 hours of residence. In some embodiments, a drug-eluting segment comprising a buprenorphine-naloxone combination formulation may release less than or equal to about 50, 45, 40, 35, 30, 25, 20, 15, 10, or 5% of the total naloxone content of the drug-eluting segment after the first 48 hours of residence. In some embodiments, a drug-eluting segment comprising a buprenorphine-naloxone combination formulation may release between about 0 and about 50% of the total naloxone content of the drug-eluting segment after the first 72 hours of residence. In some embodiments, a drug-eluting segment comprising a buprenorphine-naloxone combination formulation may release greater than or equal to about 0, 5, 10, 15, 20, 25, 30, 35, 40, or 45% of the total naloxone content of the drug-eluting segment after the first 72 hours of residence. In some embodiments, a drug-eluting segment comprising a buprenorphine-naloxone combination formulation may release less than or equal to about 50, 45, 40, 35, 30, 25, 20, 15, 10, or 5% of the total naloxone content of the drug-eluting segment after the first 72 hours of residence. In some embodiments, a drug-eluting segment comprising a buprenorphine-naloxone combination formulation may release between about 0 and about 50% of the total naloxone content of the drug-eluting segment after the first 96 hours of residence. In some embodiments, a drug-eluting segment comprising a buprenorphine-naloxone combination formulation may release greater than or equal to about 0, 5, 10, 15, 20, 25, 30, 35, 40, or 45% of the total naloxone content of the drug-eluting segment after the first 96 hours of residence. In some embodiments, a drug-eluting segment comprising a buprenorphine-naloxone combination formulation may release less than or equal to about 50, 45, 40, 35, 30, 25, 20, 15, 10, or 5% of the total naloxone content of the drug-eluting segment after the first 96 hours of residence. In some embodiments, a drug-eluting segment comprising a buprenorphine-naloxone combination formulation may release between about 0 and about 50% of the total naloxone content of the drug-eluting segment after the first 120 hours of residence. In some embodiments, a drug-eluting segment comprising a buprenorphine-naloxone combination formulation may release greater than or equal to about 0, 5, 10, 15, 20, 25, 30, 35, 40, or 45% of the total naloxone content of the drug-eluting segment after the first 120 hours of residence. In some embodiments, a drug-eluting segment comprising a buprenorphine-naloxone combination formulation may release less than or equal to about 50, 45, 40, 35, 30, 25, 20, 15, 10, or 5% of the total naloxone content of the drug-eluting segment after the first 120 hours of residence.

Carrier Polymer-Agent Segments (Drug-Eluting Segments) Formed by Co-Extruding a Combination Buprenorphine-Naloxone Formulation with a Naloxone-Only Formulation

In some embodiments, as explained above, a naloxone-only formulation may also be prepared and coextruded with the buprenorphine-naloxone combination formulation during fabrication of the carrier polymer-agent segments (i.e., drug-eluting segments) of the gastric residence system. By coextruding a naloxone-only formulation along with the buprenorphine and naloxone combination formulation, the release profile of the naloxone may be able to be better controlled. Controlling the release profile of the naloxone from the gastric residence dosage form can help minimize the possibility of mistreatment (i.e., dissolving out the naloxone and just consuming the remaining buprenorphine). Accordingly, described below is the individual naloxone formulation that can be coextruded with the buprenorphine-naloxone combination formulation immediately described above.

As used herein, the term “naloxone-only” means that the formulation does not comprise buprenorphine. The “naloxone-only” formulation may, however, include any number of other components.

Both actives, buprenorphine and naloxone, are formulated into a single segment or component of a gastric residence system—a drug-eluting segment. This is designed such that a user cannot easily manipulate the gastric residence system to remove the naloxone such that they can consume the buprenorphine only. Alternatively, the gastric residence system could be designed such that the buprenorphine and naloxone are provided in separate components of the gastric residence system. However, this would allow for easier manipulation, since a user could physically separate the two components (i.e., the buprenorphine component and the naloxone component) to consume the buprenorphine component only. Accordingly, drug-eluting segments or components described comprise both buprenorphine and naloxone.

In some embodiments, the buprenorphine and naloxone of the drug-eluting segments may release at similar rates. In some embodiments, the buprenorphine and naloxone of the drug-eluting segments may release at different rates. For example, the naloxone may release from the drug-eluting segments faster than the buprenorphine. In either case, the fact that the buprenorphine and naloxone are formulated into a single component of the gastric residence system (i.e., a drug-eluting segment) greatly minimizes a user from separating the naloxone from the buprenorphine. For example, a user might try to elute the naloxone from the gastric residence system outside of the body and consume the remaining buprenorphine. However, for gastric residence systems in which the buprenorphine and naloxone have similar release profiles, very little if any buprenorphine, if any, will remain once the naloxone has completely eluted off. For gastric residence systems in which the naloxone releases faster than the buprenorphine, a significant amount of the buprenorphine will elute off in the time it takes the naloxone to completely elute from the gastric residence system. Thus, a user will be left with a significantly smaller amount of buprenorphine than that which was originally present in the gastric residence system. In either case, abuse of the gastric residence system is minimized.

In some embodiments, a gastric residence system comprises one or more co-extruded drug-eluting segments, each of the one or more co-extruded drug-eluting segments comprising a naloxone-only formulation co-extruded with a buprenorphine-naloxone combination formulation. In some embodiments, a co-extruded drug-eluting segment comprises about 20 to about 50 wt. % buprenorphine. In some embodiments, a co-extruded drug-eluting segment comprises about 5 to about 20 wt. % total naloxone (i.e., naloxone from the naloxone-only formulation and naloxone from the buprenorphine-naloxone combination formulation). In some embodiments, a co-extruded drug-eluting segment comprises less than or equal to about 50, 45, 40, 35, 30, or 25 wt. % buprenorphine. In some embodiments, a co-extruded drug-eluting segment comprises greater than or equal to about 20, 25, 30, 35, 40, or 45 wt. % buprenorphine. In some embodiments, a co-extruded drug-eluting segment comprises less than or equal to about 20, 15, or 10 wt. % total naloxone. In some embodiments, a co-extruded drug-eluting segment comprises more than or equal to about 5, 10, or 15 wt. % total naloxone.

In some embodiments, a co-extruded drug-eluting segment may be prepared using a buprenorphine-naloxone combination formulation such as that described above, with reference to drug-eluting segments comprising only an extruded buprenorphine-naloxone combination formulation (and not a naloxone-only formulation. For example, exemplary amounts of the components for some embodiments of a buprenorphine-naloxone combination formulation used in a co-extruded drug-eluting segment are provided in the table below. The amounts are given in approximate weight percent, with the understanding that when ranges are provided, the amounts are chosen so as to add up to 100%. “Pharm. accept. salt” indicates pharmaceutically acceptable salt thereof.

| Carrier polymer-arm | | | |
|---|---|---|---|
| segment | Formulation 1 | Formulation 2 | Formulation 3 |
| Buprenorphine (or | 35-55 | 40-50 | 45 |
| pharm. accept. salt) | | | |
| Naloxone (or | 1-8 | 3-6 | 4.5 |
| pharm. accept. salt) | | | |
| PCL | 30-50 | 35-45 | 39.5 |
| PEO_{100K} | 1-15 | 5-10 | 7.0 |
| P407 | 1-5 | 2-4 | 3.0 |
| Vit E | 0.1-1 | 0.2-0.8 | 0.5 |
| SiO₂ | 0.1-1 | 0.2-0.8 | 0.5 |

Naloxone-only formulations include naloxone or a salt thereof (i.e., naloxone hydrochloride) in an amount of about 5-60, 10-55, 20-50, or 30-50 wt. % naloxone or a salt thereof. In some embodiments, a naloxone-only formulation may comprise less than or equal to about 60, about 55, about 50, about 45, about 40, about 35, about 30, about 25, about 20, about 15, or about 10 wt. % naloxone or a salt thereof. In some embodiments, a naloxone-only formulation may comprise more than or equal to about 5, about 10, about 15, about 20, about 25, about 30, about 35, about 40, about 45, about 50, or about 55 wt. % naloxone.

Naloxone-only formulations comprise about 30 wt % to 70 wt % carrier polymer. For example, the carrier polymer may comprise polycaprolactone (PCL), such as PCL having a viscosity midpoint between about 1.5 dl/g to about 2.1 dl/g, such as Corbion PC17. In some embodiments, a naloxone-only formulation comprises less than or equal to about 70, about 65, about 60, about 55, about 50, about 45, about 40, or about 35 wt % carrier polymer. In some embodiments, each drug-eluting segment comprises greater than or equal to about 30, about 35, about 40, about 45, about 50, about 55, about 60, or about 65 wt % carrier polymer.

In some embodiments, naloxone-only formulations comprise one or more excipients. For example, naloxone-only formulations can comprise one or more plasticizers, such as a poloxamer (e.g., Poloxamer 407, or “P407”). In some embodiments, a naloxone-only formulation can comprise about 0.5 to about 10 wt % plasticizer. In some embodiments, a naloxone-only formulation can comprise less than or equal to about 10, about 9, about 8, about 7, about 6, about 5, about 4, about 3, about 2, or about 1 wt % plasticizer. In some embodiments, a naloxone-only formulation can comprise more than or equal to about 0.5, about 1, about 2, about 3, about 4, about 5, about 6, about 7, about 8, or about 9 wt % plasticizer.

In some embodiments, naloxone-only formulations comprise about 0.1 wt % to about 5 wt % of Vitamin E succinate. In some embodiments, In some embodiments, a combination formulation may include less than or equal to about 5, about 4, about 3, about 2, about 1, about 0.5, or about 0.1 wt. % Vitamin E succinate. In some embodiments, a combination formulation may include more than or equal to about 0.01, about 0.1, about 0.5, about 1, about 2, about 3, or about 4 wt. % Vitamin E succinate.

In some embodiments, naloxone-only formulations comprise about 0.1 wt % to about 1 wt % of colloidal silicon dioxide (SiO₂).

Exemplary amounts of the components for some embodiments of a naloxone-only formulation used in a co-extruded drug-eluting segment are provided in the table below. The amounts are given in approximate weight percent, with the understanding that when ranges are provided, the amounts are chosen so as to add up to 100%. “Pharm. accept. salt” indicates pharmaceutically acceptable salt thereof.

| Carrier polymer-arm | | | |
|---|---|---|---|
| segment | Formulation 1 | Formulation 2 | Formulation 3 |
| Naloxone (or | 30-50 | 35-45 | 40 |
| pharm. accept. salt) | | | |
| PCL | 45-65 | 50-60 | 56 |
| P407 | 1-5 | 2-4 | 3 |
| Vit E | 0.1-1 | 0.2-0.8 | 0.5 |
| SiO₂ | 0.1-1 | 0.2-0.8 | 0.5 |

The naloxone-only formulations described above can be co-extruded with a buprenorphine-naloxone formulation described above to form a co-extruded drug-eluting segment comprising both the buprenorphine-naloxone combination formulation as well as the naloxone-only formulation. In some embodiments, the co-extruded drug-eluting segments may be formed with a ratio of buprenorphine-naloxone combination formulation to a naloxone-only formulation (“BN:NAL”) of between about 1:1 and about 20:1 BN:NAL. In some embodiments, the ratio may be about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1 BN:NAL.

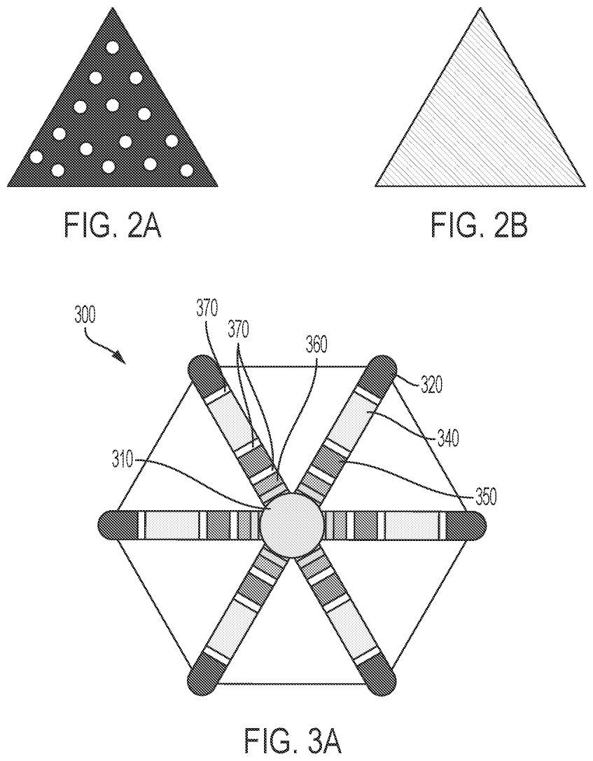

The buprenorphine-naloxone formulation may be coextruded with the naloxone-only formulation in various configurations. In some embodiments, the two formulations (or “portions”) may be extruded such that one formulation (e.g., naloxone-only formulation) is embedded within the second formulation (e.g., buprenorphine-naloxone formulation), such as that which is shown in FIG. 2A. FIG. 2A shows a cross-section of a drug-eluting segment comprising one formulation embedded throughout the second formulation. The first formulation may comprise one or more strands embedded within the second formulation. In some embodiments, the two formulations may be coextruded as a multilayered drug-eluting segment, such as that which is shown in FIG. 2B. For example, a multi-layered drug-eluting segment may comprise a plurality of layers of buprenorphine-naloxone formulation alternating with naloxone-only formulation. In some embodiments, the multi-layered drug-eluting segment may comprise more than or equal to 2, 3, 4, 5, 6, 7, 8, 9, or 10 layers. In some embodiments, the multi-layered drug-eluting segment may comprise less than or equal to 12, 10, 9, 8, 7, 6, 5, 4, or 3 layers.

Exemplary amounts of the components for some embodiments of drug-eluting segment comprising a buprenorphine-naloxone combination formulation co-extruded with a naloxone-only formulation are provided in the table below. In some embodiments, a buprenorphine-naloxone combination formulation and a naloxone-only formulation is used for the drug-eluting segment (and no additional formulations), these amounts correspond to the amounts of the drug-eluting segment. The amounts are given in approximate weight percent, with the understanding that when ranges are provided, the amounts are chosen so as to add up to 100%. “Pharm. accept. salt” indicates pharmaceutically acceptable salt thereof.

| Carrier polymer-arm | | | |
|---|---|---|---|
| segment | Formulation 1 | Formulation 2 | Formulation 3 |
| Buprenorphine (or | 30-40 | 33-39 | 36 |
| pharm. accept. salt) | | | |
| Naloxone (or | 5-15 | 10-14 | 11.6 |
| pharm. accept. salt) | | | |
| PCL | 35-50 | 40-45 | 42.8 |
| PEO_{100K} | 2-8 | 4-7 | 5.6 |
| P407 | 1-5 | 2-4 | 3 |
| Vit E | 0.1-1 | 0.2-0.8 | 0.5 |
| SiO₂ | 0.1-1 | 0.2-0.8 | 0.5 |

In some embodiments, a co-extruded drug-eluting segment comprising a buprenorphine-naloxone combination formulation co-extruded with a naloxone-only formulation may release between about 5 and about 90% of the total buprenorphine content of the co-extruded drug-eluting segment within the first 24 hours of residence. In some embodiments, a co-extruded drug-eluting segment comprising a buprenorphine-naloxone combination formulation co-extruded with a naloxone-only formulation may release greater than or equal to about 5, 10, 15, 20, 25, 30, 35, 40, 45, 50, 55, 60, 65, 70, 75, 80, or 85% of the total buprenorphine content of the co-extruded drug-eluting segment within the first 24 hours of residence. In some embodiments, a co-extruded drug-eluting segment comprising a buprenorphine-naloxone combination formulation co-extruded with a naloxone-only formulation may release less than or equal to about 90, 85, 80, 75, 70, 65, 60, 55, 50, 45, 40, 35, 30, 25, 20, 15, or 10% of the total buprenorphine content of the co-extruded drug-eluting segment within the first 24 hours of residence. In some embodiments, a co-extruded drug-eluting segment comprising a buprenorphine-naloxone combination formulation co-extruded with a naloxone-only formulation may release between about 5 and about 90% of the total buprenorphine content of the co-extruded drug-eluting segment after the first 24 hours of residence. In some embodiments, a co-extruded drug-eluting segment comprising a buprenorphine-naloxone combination formulation co-extruded with a naloxone-only formulation may release greater than or equal to about 5, 10, 15, 20, 25, 30, 35, 40, 45, 50, 55, 60, 65, 70, 75, 80, or 85% of the total buprenorphine content of the co-extruded drug-eluting segment after the first 24 hours of residence. In some embodiments, a co-extruded drug-eluting segment comprising a buprenorphine-naloxone combination formulation co-extruded with a naloxone-only formulation may release less than or equal to about 90, 85, 80, 75, 70, 65, 60, 55, 50, 45, 40, 35, 30, 25, 20, 15, or 10% of the total buprenorphine content of the co-extruded drug-eluting segment after the first 24 hours of residence. In some embodiments, a co-extruded drug-eluting segment comprising a buprenorphine-naloxone combination formulation co-extruded with a naloxone-only formulation may release between about 5 and about 70% of the total buprenorphine content of the co-extruded drug-eluting segment after the first 48 hours of residence. In some embodiments, a co-extruded drug-eluting segment comprising a buprenorphine-naloxone combination formulation co-extruded with a naloxone-only formulation may release greater than or equal to about 5, 10, 15, 20, 25, 30, 35, 40, 45, 50, 55, 60, or 65% of the total buprenorphine content of the co-extruded drug-eluting segment after the first 48 hours of residence. In some embodiments, a co-extruded drug-eluting segment comprising a buprenorphine-naloxone combination formulation co-extruded with a naloxone-only formulation may release less than or equal to about 70, 65, 60, 55, 50, 45, 40, 35, 30, 25, 20, 15, or 10% of the total buprenorphine content of the co-extruded drug-eluting segment after the first 48 hours of residence. In some embodiments, a co-extruded drug-eluting segment comprising a buprenorphine-naloxone combination formulation co-extruded with a naloxone-only formulation may release between about 5 and about 50% of the total buprenorphine content of the co-extruded drug-eluting segment after the first 72 hours of residence. In some embodiments, a co-extruded drug-eluting segment comprising a buprenorphine-naloxone combination formulation co-extruded with a naloxone-only formulation may release greater than or equal to about 5, 10, 15, 20, 25, 30, 35, 40, or 45% of the total buprenorphine content of the co-extruded drug-eluting segment after the first 72 hours of residence. In some embodiments, a co-extruded drug-eluting segment comprising a buprenorphine-naloxone combination formulation co-extruded with a naloxone-only formulation may release less than or equal to about 50, 45, 40, 35, 30, 25, 20, 15, or 10% of the total buprenorphine content of the co-extruded drug-eluting segment after the first 72 hours of residence. In some embodiments, a co-extruded drug-eluting segment comprising a buprenorphine-naloxone combination formulation co-extruded with a naloxone-only formulation may release between about 5 and about 30% of the total buprenorphine content of the co-extruded drug-eluting segment after the first 96 hours of residence. In some embodiments, a co-extruded drug-eluting segment comprising a buprenorphine-naloxone combination formulation co-extruded with a naloxone-only formulation may release greater than or equal to about 5, 10, 15, 20, or 25% of the total buprenorphine content of the co-extruded drug-eluting segment after the first 96 hours of residence. In some embodiments, a co-extruded drug-eluting segment comprising a buprenorphine-naloxone combination formulation co-extruded with a naloxone-only formulation may release less than or equal to about 30, 25, 20, 15, or 10% of the total buprenorphine content of the co-extruded drug-eluting segment after the first 96 hours of residence. In some embodiments, a co-extruded drug-eluting segment comprising a buprenorphine-naloxone combination formulation co-extruded with a naloxone-only formulation may release between about 5 and about 20% of the total buprenorphine content of the co-extruded drug-eluting segment after the first 120 hours of residence. In some embodiments, a co-extruded drug-eluting segment comprising a buprenorphine-naloxone combination formulation co-extruded with a naloxone-only formulation may release greater than or equal to about 5, 10, or 15% of the total buprenorphine content of the co-extruded drug-eluting segment after the first 120 hours of residence. In some embodiments, a co-extruded drug-eluting segment comprising a buprenorphine-naloxone combination formulation co-extruded with a naloxone-only formulation may release less than or equal to about 20, 15, or 10% of the total buprenorphine content of the co-extruded drug-eluting segment after the first 120 hours of residence.

In some embodiments, a co-extruded drug-eluting segment comprising a buprenorphine-naloxone combination formulation co-extruded with a naloxone-only formulation may release between about 5 and about 90% of the total naloxone content of the co-extruded drug-eluting segment within the first 24 hours of residence. In some embodiments, a co-extruded drug-eluting segment comprising a buprenorphine-naloxone combination formulation co-extruded with a naloxone-only formulation may release greater than or equal to about 5, 10, 15, 20, 25, 30, 35, 40, 45, 50, 55, 60, 65, 70, 75, 80, or 85% of the total naloxone content of the co-extruded drug-eluting segment within the first 24 hours of residence. In some embodiments, a co-extruded drug-eluting segment comprising a buprenorphine-naloxone combination formulation co-extruded with a naloxone-only formulation may release less than or equal to about 90, 85, 80, 75, 70, 65, 60, 55, 50, 45, 40, 35, 30, 25, 20, 15, or 10% of the total naloxone content of the co-extruded drug-eluting segment within the first 24 hours of residence. In some embodiments, a co-extruded drug-eluting segment comprising a buprenorphine-naloxone combination formulation co-extruded with a naloxone-only formulation may release between about 5 and about 90% of the total naloxone content of the co-extruded drug-eluting segment after the first 24 hours of residence. In some embodiments, a co-extruded drug-eluting segment comprising a buprenorphine-naloxone combination formulation co-extruded with a naloxone-only formulation may release greater than or equal to about 5, 10, 15, 20, 25, 30, 35, 40, 45, 50, 55, 60, 65, 70, 75, 80, or 85% of the total naloxone content of the co-extruded drug-eluting segment after the first 24 hours of residence. In some embodiments, a co-extruded drug-eluting segment comprising a buprenorphine-naloxone combination formulation co-extruded with a naloxone-only formulation may release less than or equal to about 90, 85, 80, 75, 70, 65, 60, 55, 50, 45, 40, 35, 30, 25, 20, 15, or 10% of the total naloxone content of the co-extruded drug-eluting segment after the first 24 hours of residence. In some embodiments, a co-extruded drug-eluting segment comprising a buprenorphine-naloxone combination formulation co-extruded with a naloxone-only formulation may release between about 5 and about 70% of the total naloxone content of the co-extruded drug-eluting segment after the first 48 hours of residence. In some embodiments, a co-extruded drug-eluting segment comprising a buprenorphine-naloxone combination formulation co-extruded with a naloxone-only formulation may release greater than or equal to about 5, 10, 15, 20, 25, 30, 35, 40, 45, 50, 55, 60, or 65% of the total naloxone content of the co-extruded drug-eluting segment after the first 48 hours of residence. In some embodiments, a co-extruded drug-eluting segment comprising a buprenorphine-naloxone combination formulation co-extruded with a naloxone-only formulation may release less than or equal to about 70, 65, 60, 55, 50, 45, 40, 35, 30, 25, 20, 15, or 10% of the total naloxone content of the co-extruded drug-eluting segment after the first 48 hours of residence. In some embodiments, a co-extruded drug-eluting segment comprising a buprenorphine-naloxone combination formulation co-extruded with a naloxone-only formulation may release between about 5 and about 50% of the total naloxone content of the co-extruded drug-eluting segment after the first 72 hours of residence. In some embodiments, a co-extruded drug-eluting segment comprising a buprenorphine-naloxone combination formulation co-extruded with a naloxone-only formulation may release greater than or equal to about 5, 10, 15, 20, 25, 30, 35, 40, or 45% of the total naloxone content of the co-extruded drug-eluting segment after the first 72 hours of residence. In some embodiments, a co-extruded drug-eluting segment comprising a buprenorphine-naloxone combination formulation co-extruded with a naloxone-only formulation may release less than or equal to about 50, 45, 40, 35, 30, 25, 20, 15, or 10% of the total naloxone content of the co-extruded drug-eluting segment after the first 72 hours of residence. In some embodiments, a co-extruded drug-eluting segment comprising a buprenorphine-naloxone combination formulation co-extruded with a naloxone-only formulation may release between about 5 and about 30% of the total naloxone content of the co-extruded drug-eluting segment after the first 96 hours of residence. In some embodiments, a co-extruded drug-eluting segment comprising a buprenorphine-naloxone combination formulation co-extruded with a naloxone-only formulation may release greater than or equal to about 5, 10, 15, 20, or 25% of the total naloxone content of the co-extruded drug-eluting segment after the first 96 hours of residence. In some embodiments, a co-extruded drug-eluting segment comprising a buprenorphine-naloxone combination formulation co-extruded with a naloxone-only formulation may release less than or equal to about 30, 25, 20, 15, or 10% of the total naloxone content of the co-extruded drug-eluting segment after the first 96 hours of residence. In some embodiments, a co-extruded drug-eluting segment comprising a buprenorphine-naloxone combination formulation co-extruded with a naloxone-only formulation may release between about 5 and about 20% of the total naloxone content of the co-extruded drug-eluting segment after the first 120 hours of residence. In some embodiments, a co-extruded drug-eluting segment comprising a buprenorphine-naloxone combination formulation co-extruded with a naloxone-only formulation may release greater than or equal to about 5, 10, or 15% of the total naloxone content of the co-extruded drug-eluting segment after the first 120 hours of residence. In some embodiments, a co-extruded drug-eluting segment comprising a buprenorphine-naloxone combination formulation co-extruded with a naloxone-only formulation may release less than or equal to about 20, 15, or 10% of the total naloxone content of the co-extruded drug-eluting segment after the first 120 hours of residence.

### Rate-Modulating Polymer Films

Release-rate modulating polymer films can be coated onto components of gastric residence systems which release agents, such as drugs. In some embodiments, a release-rate modulating polymer film controls burst release of the active ingredient(s) of a drug-eluting segment and linearizes the release of the active ingredient(s). Components coated with the release-rate modulating polymer films disclosed herein have substantially the same release-rate properties before and after exposure to heat which occurs during heat-assisted assembly of a gastric residence system. The composition, parameters, advantages, features, applications and release profiles of release-rate modulating polymer films are disclosed in International Patent Application PCT/US2020/059541, which are hereby incorporated in its entirety. In some embodiments, one or more segments of the composite arms (such as a composite arm including the drug-eluting segment or a composite arm excluding the drug-eluting segment) are coated with a release rate-modulating film. In some embodiments, the drug-eluting segment is coated with a release rate-modulating film.

Various polymers can be used to form the release-rate modulating polymer films, including PCL. In some embodiments, the release-rate modulating polymer films comprises about 70 wt % to about 80 wt % PCL. In some embodiments, the release-rate modulating polymer films comprises about 73 wt % to about 77 wt % PCL. In some embodiments, the release-rate modulating polymer films comprises about 73.5 wt % PCL. In some embodiments, the release rate-modulating polymer film comprises less than or equal to about 80, 79, 78, 77, 76, 75, 74, 73, 72, or 71 wt % PCL. In some embodiments, the release rate-modulating polymer film comprises greater than or equal to 70, 71, 72, 73, 74, 75, 76, 77, 78, or 79 wt % PCL.

Other excipients can be added to the carrier polymers to modulate the release of agent, such as copovidone (VA64). In some embodiments, the release-rate modulating polymer films comprises about 20 wt % to about 30 wt % VA64. In some embodiments, the release-rate modulating polymer films comprises about 23 wt % to about 27 wt % VA64. In some embodiments, the release-rate modulating polymer films comprises about 24.5 wt % VA64. In some embodiments, a release rate-modulating polymer film comprises less than or equal to 30, 29, 28, 27, 26, 25, 24, 23, 22, or 21 wt % VA64. In some embodiments, a release rate-modulating polymer film comprises more than or equal to 20, 21, 22, 23, 24, 25, 26, 27, 28, or 29 wt % VA64.

In some embodiments, a release rate-modulating polymer film comprises 1-5 wt % magnesium stearate. In some embodiments, a release rate-modulating film comprises less than or equal to 5, 4, 3, or 2 wt % magnesium stearate. In some embodiments, a release rate-modulating film comprises more than or equal to 1, 2, 3, or 4 wt % magnesium stearate.

### Gastric Residence Time

The gastric residence time of the system is controlled by the degradation or weakening, or breakage, rate of the time-dependent polymeric linker in the gastric residence system. Faster degradation or weakening, or breakage of the time-dependent polymeric linker results in faster passage of the system from the stomach. The residence time of the gastric residence system is defined as the time between administration of the system to the stomach and exit of the system from the stomach. In one embodiment, the gastric residence system has a residence time of about 24 hours, or up to about 24 hours. In one embodiment, the gastric residence system has a residence time of about 48 hours, or up to about 48 hours. In one embodiment, the gastric residence system has a residence time of about 72 hours, or up to about 72 hours. In one embodiment, the gastric residence system has a residence time of about 96 hours, or up to about 96 hours. In one embodiment, the gastric residence system has a residence time of about 5 days, or up to about 5 days. In one embodiment, the gastric residence system has a residence time of about 6 days, or up to about 6 days. In one embodiment, the gastric residence system has a residence time of about 7 days (about one week), or up to about 7 days (about one week). In one embodiment, the gastric residence system has a residence time of about 10 days, or up to about 10 days. In one embodiment, the gastric residence system has a residence time of about 14 days (about two weeks), or up to about 14 days (about two weeks).

In one embodiment, the gastric residence system has a residence time between about 24 hours and about 7 days. In one embodiment, the gastric residence system has a residence time between about 48 hours and about 7 days. In one embodiment, the gastric residence system has a residence time between about 72 hours and about 7 days. In one embodiment, the gastric residence system has a residence time between about 96 hours and about 7 days. In one embodiment, the gastric residence system has a residence time between about 5 days and about 7 days. In one embodiment, the gastric residence system has a residence time between about 6 days and about 7 days.

In one embodiment, the gastric residence system has a residence time between about 24 hours and about 10 days. In one embodiment, the gastric residence system has a residence time between about 48 hours and about 10 days. In one embodiment, the gastric residence system has a residence time between about 72 hours and about 10 days. In one embodiment, the gastric residence system has a residence time between about 96 hours and about 10 days. In one embodiment, the gastric residence system has a residence time between about 5 days and about 10 days. In one embodiment, the gastric residence system has a residence time between about 6 days and about 10 days. In one embodiment, the gastric residence system has a residence time between about 7 days and about 10 days.

In one embodiment, the gastric residence system has a residence time between about 24 hours and about 14 days. In one embodiment, the gastric residence system has a residence time between about 48 hours and about 14 days. In one embodiment, the gastric residence system has a residence time between about 72 hours and about 14 days. In one embodiment, the gastric residence system has a residence time between about 96 hours and about 14 days. In one embodiment, the gastric residence system has a residence time between about 5 days and about 14 days. In one embodiment, the gastric residence system has a residence time between about 6 days and about 14 days. In one embodiment, the gastric residence system has a residence time between about 7 days and about 14 days. In one embodiment, the gastric residence system has a residence time between about 10 days and about 14 days.

The gastric residence system releases a therapeutically effective amount of agent (or salt thereof) during at least a portion of the residence time or residence period during which the system resides in the stomach. In one embodiment, the system releases a therapeutically effective amount of agent (or salt thereof) during at least about 25% of the residence time. In one embodiment, the system releases a therapeutically effective amount of agent (or salt thereof) during at least about 50% of the residence time. In one embodiment, the system releases a therapeutically effective amount of agent (or salt thereof) during at least about 60% of the residence time. In one embodiment, the system releases a therapeutically effective amount of agent (or salt thereof) during at least about 70% of the residence time. In one embodiment, the system releases a therapeutically effective amount of agent (or salt thereof) during at least about 75% of the residence time. In one embodiment, the system releases a therapeutically effective amount of agent (or salt thereof) during at least about 80% of the residence time. In one embodiment, the system releases a therapeutically effective amount of agent (or salt thereof) during at least about 85% of the residence time. In one embodiment, the system releases a therapeutically effective amount of agent (or salt thereof) during at least about 90% of the residence time. In one embodiment, the system releases a therapeutically effective amount of agent (or salt thereof) during at least about 95% of the residence time. In one embodiment, the system releases a therapeutically effective amount of agent (or salt thereof) during at least about 98% of the residence time. In one embodiment, the system releases a therapeutically effective amount of agent (or salt thereof) during at least about 99% of the residence time.

### Gastric Residence Systems Comprising Buprenorphine and Naloxone

In some embodiments, a stellate-shaped dosage form for administration of buprenorphine and naloxone can comprise arms, which arms in turn comprise 1) a carrier polymer-agent arm segment; 2) one or more enteric linkers; 3) one or more time-dependent linkers; 4) an inactive segment; and/or 5) other optional spacers. The arms are connected to an elastomeric core in a stellate device arrangement. Typically, six arms are used for a stellate dosage form. In some embodiments, wherein six arms are used for a stellate dosage form, any one of 1, 2, 3, 4, 5, or 6 arms comprise a carrier polymer-agent arm segment. In some embodiments, wherein six arms are used for a stellate dosage form, 3 arms comprise the carrier polymer-agent arm segment. In some embodiments, wherein six arms are used for a stellate dosage form, 6 arms comprise the carrier polymer-agent arm segment. In some embodiments, wherein six arms are used for a stellate dosage form, 2 arms comprise the carrier polymer-agent arm segment. In some embodiments, wherein six arms are used for a stellate dosage form, 4 arms comprise the carrier polymer-agent arm segment.

The carrier polymer-agent arm segments of the buprenorphine and naloxone dosage form can comprise buprenorphine (or a pharmaceutically acceptable salt thereof), naloxone (or a pharmaceutically acceptable salt thereof), polycaprolactone, polyethylene oxide (PEO100K), poloxamer 407 (P407), silica (SiO₂), and vitamin E succinate (vitE). In some embodiments, typically six arms are used for a stellate dosage form, and either 1, 2, 3, 4, 5 or 6 of the arms comprise the carrier polymer-agent arm segment. In some embodiments, 3 of the arms comprise the carrier polymer-agent arm segment. In some embodiments, 6 of the arms comprise the carrier polymer-agent arm segment. In some embodiments, 2 of the arms can comprises the carrier polymer-agent arm segment. In some embodiments, 4 of the arms can comprises the carrier polymer-agent arm segment. In some embodiments, arms that do not comprises a carrier agent-polymer segment comprise an inactive segment instead. In some embodiments, the total amount of agent contained in the dosage form is 1, 2, 3, 4, 5, or 6 times the amount of agent contained in a single arm. In some embodiments, the total amount of agent contained in the dosage form is 3 times the amount of agent contained in a single arm. In some embodiments, the total amount of agent contained in the dosage form is 6 times the amount of agent contained in a single arm. In some embodiments, the total amount of agent contained in the dosage form is 4 times the amount of agent contained in a single arm. The total amount of weight of buprenorphine, pharmaceutically acceptable salt of buprenorphine, or salt of buprenorphine in the stellate dosage form can range from 10 mg to about 150 mg of buprenorphine. In some embodiments, a dosage form for administration of buprenorphine and naloxone comprises a gastric residence system comprising about 40 mg to about 115 mg of buprenorphine. In some embodiments, a dosage form for administration of buprenorphine and naloxone comprises a gastric residence system comprising less than or equal to about 150, about 140, about 130, about 120, about 110, about 100, about 90, about 80, about 70, about 60, about 50, about 40, about 30, or about 20 mg of buprenorphine. In some embodiments, a dosage form for administration of buprenorphine and naloxone comprises a gastric residence system comprising greater than or equal to about 10, about 20, about 30, about 40, about 50, about 60, about 70, about 80, about 90, about 100, about 110, about 120, about 130, or about 140 mg buprenorphine. The total amount of weight of naloxone, pharmaceutically acceptable salt of naloxone, or salt of naloxone in the stellate dosage form can range from about 1 mg to about 60 mg naloxone. In some embodiments, a dosage form for administration of buprenorphine and naloxone comprises a gastric residence system comprising about 3.5 mg to about 10 mg naloxone. In some embodiments, a dosage form for administration of buprenorphine and naloxone comprises a gastric residence system comprising less than or equal to about 60, about 55, about 50, about 45, about 40, about 35, about 30, about 25, about 20, about 15, about 10, or about 5 mg naloxone. In some embodiments, a dosage form for administration of buprenorphine and naloxone comprises a gastric residence system comprising greater than or equal to about 1, about 5, about 10, about 15, about 20, about 25, about 30, about 35, about 40, about 45, about 50, or about 55 mg naloxone.

The inactive arm segments of the dosage form can comprise polycaprolactone (PCL), a radiopaque substance, and optionally coloring. The polycaprolactone used can be from about 1.5 dL/g to about 1.9 dL/g viscosity, such as about 1.7 dL/g. The radiopaque substance can be (BiO)₂CO₃. Any pharmaceutically acceptable coloring agent can be used. An example of coloring that can be used includes FD&C Blue #5.

The enteric disintegrating matrices of the dosage form can comprise polycaprolactone (PCL), hydroxypropyl methyl cellulose acetate succinate (HPMCAS), poloxamer 407 (P407), and optionally coloring. The polycaprolactone used can be from about 1.5 dL/g to about 1.9 dL/g viscosity, such as about 1.7 dL/g. The HPMCAS used can be MG grade (M grade: about 7-11% acetyl content, about 10-14% succinoyl content, about 21-25% methoxyl content, about 5-9% hydroxypropoxy content; G grade: granular). Any pharmaceutically acceptable coloring agent can be used. An example of coloring that can be used includes ferrosoferric oxide.

The time dependent disintegrating matrices of the buprenorphine-naloxone dosage form can comprise poly(D,L-lactide-co-glycolide) (PLGA), polyethylene oxide (PEO), and optionally coloring. The poly(D,L-lactide-co-glycolide) can be in about a 75:25 lactide:glycolide molar ratio with a viscosity range of about 0.32-0.44 dL/g. The polyethylene oxide used can be from about 60,000 MW to about 125,000 MW, such as about 90,000 MW to 110,000 MW, or about 100,000 MW.

The central elastomer of the buprenorphine-naloxone dosage form can be of about 40 A to about 60 A durometer, such as about 45 A to about 55 A durometer, or about 50 A durometer. The central elastomer can be made from liquid silicone rubber; e.g., the central elastomer can comprise cured liquid silicone rubber.

In some embodiments, an assembled arm of a buprenorphine-naloxone gastric residence dosage form may be attached to the central elastomer at a polymeric linker segment. The polymeric linker segment can serve as an attachment point between the assembled arm and the central elastomer. In some embodiments, the polymeric linker segment may attach the central elastomer to a time-dependent disintegrating matrix segment of the arm. In some embodiments, the polymeric linker comprises polycaprolactone such as PCL having a viscosity midpoint between about 1.5 dl/g to about 2.1 dl/g, such as Corbion PC17. In some embodiments, the polymeric linker is adjacent to the central elastomer.

In some embodiments, the gastric residence system further comprises a release rate-modulating film comprising about 73.5 wt % of polycaprolactone (PCL), such as PCL having a viscosity midpoint between about 1.5 dl/g to about 2.1 dl/g, such as Corbion PC17. In some embodiments, the release rate-modulating film further comprises about 24.5 wt % of copovidone, such as VA64. In some embodiments, the release rate-modulating film further comprises about 2.0 wt % of Mg stearate.

Exemplary amounts for the various components of the dosage form are provided in the table below. The amounts are given in approximate weight percent, with the understanding that when ranges are provided, the amounts are chosen so as to add up to 100%.

| Carrier polymer- | | | |
|---|---|---|---|
| arm segment | Formulation 1 | Formulation 2 | Formulation 3 |
| Buprenorphine (or | 35-50 | 40-45 | 42 |
| pharm. accept. salt) | | | |
| Naloxone (or | 1-7 | 2-6 | 3.7 |
| pharm. accept. salt) | | | |
| PCL | 35-50 | 40-45 | 43.3 |
| PEO_{100K} | 1-15 | 5-10 | 7.0 |
| P407 | 1-5 | 2-4 | 3.0 |
| Vit E | 0.1-1 | 0.2-0.8 | 0.5 |
| SiO₂ | 0.1-1 | 0.2-0.8 | 0.5 |
| Inactive spacer | Formulation 1 | Formulation 2 | Formulation 3 |
| PCL | 61-71 | 64-69 | 66.45 |
| VA64 | 27-37 | 30-34 | 32 |
| P407 | 0.2-4 | 0.5-2.5 | 1.5 |
| coloring (optional) | 0.005-0.2 | 0.01-0.1 | 0.05 (e.g. Blue #1) |
| Enteric | | | |
| disintegrating | | | |
| matrix | Formulation 1 | Formulation 2 | Formulation 3 |
| PCL | 30-40 | 32-37 | 33.95 |
| HPMCAS | 60-70 | 62-66 | 63.95 |
| P407 | 0.5-5 | 1-3 | 2 |
| coloring (optional) | 0.01-0.5 | 0.05-0.15 | 0.1 (e.g. E172) |
| Time-dependent | | | |
| disintegrating | | | |
| matrix | Formulation 1 | Formulation 2 | Formulation 3 |
| PCL | 40-50 | 43-47 | 44.95 |
| PDLG5004A | 30-40 | 33-37 | 35 |
| PDLG5004 | 10-25 | 15-20 | 18 |
| PEO(100k) | 0.5-5 | 1-3 | 2 |
| coloring (optional) | 0.005-0.2 | 0.01-0.1 | 0.05 (e.g. E172) |
| ODMTEP | | | |
| disintegrating | | | |
| matrix | Formulation 1 | Formulation 2 | Formulation 3 |
| PCL | 25-35 | 28-32 | 30 |
| HPMCAS | 60-70 | 63-67 | 64.9 |
| Stearic acid | 1-5 | 2-3 | 2.5 |
| Propylene Glycol | 1-5 | 2-3 | 2.5 |

The assembled arms can comprise 1) a polymeric linker segment; 2) a first disintegrating matrix segment; 3) a second disintegrating matrix segment; 4) a drug eluting segment, wherein the drug eluting segment comprises a carrier polymer, buprenorphine or a salt thereof, and naloxone or a salt thereof, 5) a third disintegrating matrix segment, which can be arranged in various orders. One such order is, starting from the proximal end which is attached to the central elastomer, and proceeding to the distal end: (a polymeric linker segment) (a first disintegrating matrix segment) (a second disintegrating matrix segment) (a drug eluting segment) (a third disintegrating matrix segment).

In some embodiments, one or more arms of a gastric residence system may not comprise a drug-eluting segment. For example, an assembled arm without a drug-eluting segment can comprise 1) a polymeric linker segment; 2) a first disintegrating matrix segment; 3) a second disintegrating matrix segment; 4) a first inactive segment; and 5) a third disintegrating matrix segment, which can be arranged in various orders. One such order is, starting from the proximal end which is attached to the central elastomer, and proceeding to the distal end: (a polymeric linker segment) (a first disintegrating matrix segment) (a second disintegrating matrix segment) (a first inert segment) (a third disintegrating matrix segment).

Approximate dimensions for the length of the segments on each arm are provided below. Optional rPCL spacers (inert segments) of about 0.2-2 mm width, such as about 0.5 mm width, can be inserted between any two components below, or added to the outer tip of the assembled arm, or between the inner tip of the assembled arm and the elastomeric core. It will be appreciated that this embodiment of the assembled arm lacks a drug-eluting segment, and can be used when it is desired to use one or more non-drug-eluting arms for the dosage form.

| Component | Dimension set 1 | | Dimension set 2 | | Dimension set 3 | |
|---|---|---|---|---|---|---|
| Carrier polymer- | 5-12 | mm | 8-9 | mm | 8.4 | mm |
| agent segment | | | | | | |
| Inactive segment | 5-12 | mm | 8-9 | mm | 8.4 | mm |
| Timed disintegrating | 0.25-5 | mm | 0.5-2 | mm | 1.0 | mm |
| matrix (First | | | | | | |
| disintegrating | | | | | | |
| segment) | | | | | | |
| Enteric | 0.5-5 | mm | 1-3 | mm | 1.85 | mm |
| disintegrating matrix | | | | | | |
| (Second | | | | | | |
| disintegrating | | | | | | |
| segment) | | | | | | |
| Third disintegrating | 1-6 | mm | 3-5 | mm | 4 | mm |
| matrix | | | | | | |
| Polymeric Linker | 0.5-2.5 | mm | 1-1.5 | mm | 1.25 | mm |
| Segment | | | | | | |

### Exemplary Gastric Residence Systems

The following gastric residence systems are exemplary to better illustrate certain embodiments of the system described herein. As these examples are only exemplary, they are not intended to limit the gastric residence system described herein. One skilled in the art, in view of the provided disclosure, would be able to contemplate additional configurations of the gastric residence system.

In some embodiments, the gastric residence system comprises at least one arm including a drug-eluting segment, wherein the arm comprises: (a) a polymeric linker segment as described in any of the embodiments above; (b) a timed disintegrating matrix as described in any of the embodiments above, (c) a first inert segment as described in any of the embodiments of inert segment above, (d) an enteric disintegrating matrix as described in any of the embodiments above, (e) a second inert segment as described in any of the embodiments of inert segment above, (f) a drug-eluting segment as described in any of the embodiments described above, (g) a third inert segment as described in any of the embodiments of inert segment above, and (h) a third disintegrating matrix as described in any of the embodiments above. The polymeric linker segment can be attached to a central elastomer.

In some embodiments, the gastric residence system comprises at least one arm including a drug eluting segment, wherein the arm can be attached to a central elastomer, and the arm comprises one or more of: (a) a polymeric linker segment; (b) a timed disintegrating matrix, (c) a first inert segment, (d) an enteric disintegrating matrix, (e) a second inert segment, (f) a drug-eluting segment, (g) a third inert segment, and (h) a third disintegrating matrix, wherein:
the central elastomer comprises liquid silicone rubber (LSR) having a hardness of about 40 to about 65 durometer;
(a) the polymeric linker segment comprises 100 wt % PCL;
(b) the timed disintegrating matrix comprises about 40 wt % to about 50 wt % PCL, about 30 wt % to about 40 wt % of acid terminated copolymer of DL-lactide and glycolide (50/50 molar ratio) having a viscosity midpoint of about 0.4 dl/g, about 10 wt % to about 25 wt % of copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172;
(c) the first inert segment comprises about 65 wt % to about 75 wt % PCL, and about 25 wt % to about 35 wt % (BiO)₂CO₃;
(d) the enteric disintegrating matrix comprises about 60 wt % to about 70 wt % HPMCAS, about 30 wt % to about 40 wt % PCL, and about 0.5 wt % to about 5 wt % poloxamer (such as P407) and optionally about 0.01 wt % to about 0.5 wt % iron oxide (such as E172);
(e) the second inert segment comprises about 65 wt % to about 75 wt % PCL, and about 25 wt % to about 35 wt % (BiO)₂CO₃;
(f) the drug-eluting segment comprises about 35 wt % to about 50 wt % of buprenorphine, about 1 wt % to about 7 wt % naloxone; about 35 wt % to about 50 wt % of PCL, about 1 wt % to about 15 wt % of PEO100K, about 1 wt % to about 5 wt % of P407, about 0.1 wt % to about 1 wt % of Vitamin E succinate, and about 0.1 wt % to about 1 wt % of SiO₂;
(g) the third inert segment comprises about 65 wt % to about 75 wt % PCL, and about 25 wt % to about 35 wt % (BiO)₂CO₃; and/or
(h) the third disintegrating matrix comprises about 60 wt % to about 70 wt % HPMCAS, about 25 wt % to about 35 wt % PCL, about 1 wt % to about 5 wt % propylene glycol and about 1 wt % to about 5 wt % stearic acid and optionally about 0.01 wt % to about 0.5 wt % iron oxide.

In some embodiments, the gastric residence system comprises at least one arm including a drug eluting segment, wherein the arm can be attached to a central elastomer, and the arm comprises one or more of: (a) a polymeric linker segment; (b) a timed disintegrating matrix, (c) a first inert segment, (d) an enteric disintegrating matrix, (e) a second inert segment, (f) a drug-eluting segment, (g) a third inert segment, and (h) a third disintegrating matrix, wherein:
the central elastomer comprises liquid silicone rubber (LSR) having a hardness of about 45 to about 55 durometer;
(a) the polymeric linker segment comprises 100 wt % PCL;
(b) the time-dependent disintegrating matrix comprises about 43 wt % to about 47 wt % PCL, about 33 wt % to about 37 wt % of acid terminated copolymer of DL-lactide and glycolide (50/50 molar ratio) having a viscosity midpoint of about 0.4 dl/g, about 15 wt % to about 20 wt % of copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172;
(c) the first inert segment comprises about 68 wt % to about 72 wt % PCL, and about 28 wt % to about 32 wt % (BiO)₂CO₃;
(d) the enteric disintegrating matrix comprises about 62 wt % to about 66 wt % HPMCAS, about 32 wt % to about 37 wt % PCL, and about 1 wt % to about 3 wt % poloxamer (such as P407) and optionally about 0.05 wt % to about 0.15 wt % iron oxide (such as E172);
(e) the second inert segment comprises about 68 wt % to about 72 wt % PCL, and about 28 wt % to about 32 wt % (BiO)₂CO₃;
(f) the drug-eluting segment comprises about 40 wt % to about 45 wt % of buprenorphine, about 2 wt % to about 6 wt % naloxone; about 40 wt % to about 45 wt % of PCL, about 5 wt % to about 10 wt % PEO100K, about 2 wt % to about 4 wt % of P407, about 0.2 wt % to about 0.8 wt % of Vitamin E succinate, and about 0.2 wt % to about 0.8 wt % of SiO₂;
(g) the third inert segment comprises about 68 wt % to about 72 wt % PCL, and about 28 wt % to about 32 wt % (BiO)₂CO₃; and/or
(h) the third disintegrating matrix comprises about 63 wt % to about 67 wt % HPMCAS, about 28 wt % to about 32 wt % PCL, about 2 wt % to about 3 wt % propylene glycol and about 2 wt % to about 3 wt % stearic acid.

In some embodiments, the gastric residence system comprises at least one arm including a drug eluting segment, wherein the arm can be attached to a central elastomer, and the arm comprises one or more of: (a) a polymeric linker segment; (b) a timed disintegrating matrix, (c) a first inert segment, (d) an enteric disintegrating matrix, (e) a second inert segment, (f) a drug-eluting segment, (g) a third inert segment, and (h) a third disintegrating matrix, wherein:
the central elastomer comprises liquid silicone rubber (LSR) having a hardness of about 50 durometer;
(a) the polymeric linker segment comprises 100 wt % PCL;
(b) time-dependent disintegrating matrix, the time-dependent disintegrating matrix comprises about 44.95 wt % PCL, about 35 wt % of acid terminated copolymer of DL-lactide and glycolide (50/50 molar ratio) having a viscosity midpoint of about 0.4 dl/g, about 18 wt % of copolymer of DL-lactide and glycolide (50/50 molar ratio) having a viscosity midpoint of about 0.4 dl/g, about 2 wt % of polyethylene glycol 100 k and about 0.05 wt % color-absorbing dye E172;
(c) the first inert segment comprises about 70 wt % PCL, and about 30 wt % (BiO)₂CO₃;
(d) the enteric disintegrating matrix comprises about 63.95 wt % HPMCAS, about 33.95 wt % PCL, and about 2 wt % poloxamer (such as P407) and about 0.1 wt % iron oxide (such as E172);
(e) the second inert segment comprises about 70 wt % PCL, and about 30 wt % (BiO)₂CO₃;
(f) drug-eluting segment comprises about 42 wt % of buprenorphine, about 3.7 wt % naloxone; about 7 wt % PEO100K; about 43.3 wt % of PCL, about 3.0 wt % of P407, about 0.5 wt % of Vitamin E succinate, and about 0.5 wt % of SiO₂;
(g) the third inert segment comprises about 70 wt % PCL, and about 30 wt % (BiO)₂CO₃; and/or
(h) the third disintegrating matrix comprises 64.9 wt % HPMCAS, about 30 wt % PCL, about 2.5 wt % propylene glycol and about 2.5 wt % stearic acid and optionally about 0.1 wt % iron oxide, for example about 0.025% ferrosoferric oxide and about 0.075% FD&C Red 40.

In some embodiments, the gastric residence system comprises at least one arm excluding a drug eluting segment, wherein the arm can be attached to a central elastomer, and the arm comprises one or more of: (a) a polymeric linker segment; (b) a timed disintegrating matrix, (c) a first inert segment, (d) an enteric disintegrating matrix, (e) a second inert segment, (f) a third inert segment, (g) a third inert segment; and (h) a third disintegrating matrix, wherein:
the central elastomer comprises liquid silicone rubber (LSR) having a hardness of about 40 to about 65 durometer;
(a) the polymeric linker segment comprises 100 wt % PCL;
(b) the timed disintegrating matrix comprises about 40 wt % to about 50 wt % PCL, about 30 wt % to about 40 wt % of acid terminated copolymer of DL-lactide and glycolide (50/50 molar ratio) having a viscosity midpoint of about 0.4 dl/g, about 10 wt % to about 25 wt % of copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172;
(c) the first inert segment comprises about 65 wt % to about 75 wt % PCL, and about 25 wt % to about 35 wt % (BiO)₂CO₃;
(d) the enteric disintegrating matrix comprises about 60 wt % to about 70 wt % HPMCAS, about 30 wt % to about 40 wt % PCL, and about 0.5 wt % to about 5 wt % poloxamer (such as P407) and optionally about 0.01 wt % to about 0.5 wt % iron oxide (such as E172);
(e) the second inert segment comprises about 65 wt % to about 75 wt % PCL, and about 25 wt % to about 35 wt % (BiO)₂CO₃;
(f) the third inert segment comprises about 61 wt % to about 71 wt % of PCL, about 27 wt % to about 37 wt % of VA64, about 0.2 wt % to about 4 wt % of P407, and about 0.005 wt % to about 0.2 wt % of a pigment or coloring;
(g) the fourth inert segment comprises about 65 wt % to about 75 wt % PCL, and about 25 wt % to about 35 wt % (BiO)₂CO₃; and/or
(h) the third disintegrating matrix comprises about 60 wt % to about 70 wt % HPMCAS, about 25 wt % to about 35 wt % PCL, about 1 wt % to about 5 wt % propylene glycol and about 1 wt % to about 5 wt % stearic acid and optionally about 0.01 wt % to about 0.5 wt % iron oxide.

In some embodiments, the gastric residence system comprises at least one arm excluding a drug eluting segment, wherein the arm can be attached to a central elastomer, and the arm comprises one or more of: (a) a polymeric linker segment; (b) a timed disintegrating matrix, (c) a first inert segment, (d) an enteric disintegrating matrix, (e) a second inert segment, (f) a third inert segment, (g) a third inert segment; and (h) a third disintegrating matrix, wherein:
the central elastomer comprises liquid silicone rubber (LSR) having a hardness of about 45 to about 55 durometer;
(a) the polymeric linker segment comprises 100 wt % PCL;
(b) the time-dependent disintegrating matrix comprises about 43 wt % to about 47 wt % PCL, about 33 wt % to about 37 wt % of acid terminated copolymer of DL-lactide and glycolide (50/50 molar ratio) having a viscosity midpoint of about 0.4 dl/g, about 15 wt % to about 20 wt % of copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172;
(c) the first inert segment comprises about 68 wt % to about 72 wt % PCL, and about 28 wt % to about 32 wt % (BiO)₂CO₃;
(d) the enteric disintegrating matrix comprises about 62 wt % to about 66 wt % HPMCAS, about 32 wt % to about 37 wt % PCL, and about 1 wt % to about 3 wt % poloxamer (such as P407) and optionally about 0.05 wt % to about 0.15 wt % iron oxide (such as E172);
(e) the second inert segment comprises about 68 wt % to about 72 wt % PCL, and about 28 wt % to about 32 wt % (BIO)₂CO₃;
(f) the third inert segment comprises about 64 wt % to about 69 wt % of PCL, about 30 wt % to about 34 wt % of VA64, about 0.5 wt % to about 2.5 wt % of P407, and about 0.01 wt % to about 0.1 wt % of a pigment or coloring;
(g) the fourth inert segment comprises about 68 wt % to about 72 wt % PCL, and about 28 wt % to about 32 wt % (BiO)₂CO₃; and/or
(h) the third disintegrating matrix comprises about 63 wt % to about 67 wt % HPMCAS, about 28 wt % to about 32 wt % PCL, about 2 wt % to about 3 wt % propylene glycol and about 2 wt % to about 3 wt % stearic acid.

In some embodiments, the gastric residence system comprises at least one arm excluding a drug eluting segment, wherein the arm can be attached to a central elastomer, and the arm comprises one or more of: (a) a polymeric segment; (b) a timed disintegrating matrix, (c) a first inert segment, (d) an enteric disintegrating matrix, (e) a second inert segment, (f) a third inert segment, (g) a third inert segment; and (h) a third disintegrating matrix, wherein:
the central elastomer comprises liquid silicone rubber (LSR) having a hardness of about 50 durometer;
(a) the polymeric linker segment comprises 100 wt % PCL;
(b) time-dependent disintegrating matrix, the time-dependent disintegrating matrix comprises about 44.95 wt % PCL, about 35 wt % of acid terminated copolymer of DL-lactide and glycolide (50/50 molar ratio) having a viscosity midpoint of about 0.4 dl/g, about 18 wt % of copolymer of DL-lactide and glycolide (50/50 molar ratio) having a viscosity midpoint of about 0.4 dl/g, about 2 wt % of polyethylene glycol 100 k and about 0.05 wt % color-absorbing dye E172;
(c) the first inert segment comprises about 70 wt % PCL, and about 30 wt % (BiO)₂CO₃;
(d) the enteric disintegrating matrix comprises about 63.95 wt % HPMCAS, about 33.95 wt % PCL, and about 2 wt % poloxamer (such as P407) and about 0.1 wt % iron oxide (such as E172);
(e) the second inert segment comprises about 70 wt % PCL, and about 30 wt % (BiO)₂CO₃;
(f) the third inert segment comprises about 6.45 wt % of PCL, about 32 wt % of VA64, about 1.5 wt % of P407, and about 0.05 wt % of a pigment or coloring;
(g) the fourth inert segment comprises about 70 wt % PCL, and about 30 wt % (BiO)₂CO₃; and/or
(h) the third disintegrating matrix comprises 64.9 wt % HPMCAS, about 30 wt % PCL, about 2.5 wt % propylene glycol and about 2.5 wt % stearic acid and optionally about 0.1 wt % iron oxide, for example about 0.025% ferrosoferric oxide and about 0.075% FD&C Red 40.

In any of the above-described embodiments, the arm can be attached to the central elastomer at the polymeric linker segment. That is, the polymeric linker segment is the proximal end of the arm.

In some embodiments, the dosage form for administration of buprenorphine and naloxone comprises a gastric residence system, wherein the gastric residence system comprises between one and five inactive segments (e.g., one per arm, for a total of one to five arms comprising an inactive segment). In some embodiments, the gastric residence system comprises a first inactive segment comprising about 66.45 wt % of polycaprolactone (PCL), such as PCL having a viscosity midpoint between about 1.5 dl/g to about 2.1 dl/g, such as Corbion PC17. In some embodiments, the gastric residence system comprises a first inactive segment comprising about, about 32.0 wt % of copovidone, such as VA64. In some embodiments, the gastric residence system comprises a first inactive segment comprising about 1.5 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407 (P407). In some embodiments, the gastric residence system comprises a first inactive segment comprising about 0.005 wt % of iron oxide, such as E172.

In some embodiments, a gastric residence system dosage form for administration of one or more agents can comprise a radiopaque segment, where the segment comprises about 70 wt % of polycaprolactone (PCL), such as PCL having a viscosity midpoint between about 1.5 dl/g to about 2.1 dl/g, such as Corbion PC17. In some embodiments, the gastric residence system comprises a radiopaque segment comprising about 30 wt % of (BiO)₂CO. In some embodiments, the gastric residence system comprises a radiopaque segment comprising about 70 wt % of Corbion PC17, and about 30 wt % of (BiO)₂CO₃.

In some embodiments, a gastric residence system dosage form for administration of buprenorphine and naloxone comprises a central elastomer, and a drug-eluting segment comprising about 20 mg of buprenorphine and about 1.75 mg naloxone. In some embodiments, the gastric residence system further comprises a release rate-modulating film comprising about 73.5 wt % of polycaprolactone (PCL), such as PCL having a viscosity midpoint between about 1.5 dl/g to about 2.1 dl/g, such as Corbion PC17. In some embodiments, the release rate-modulating film further comprises about 24.5 wt % of copovidone, such as VA64. In some embodiments, the release rate-modulating film also comprises about 2 wt % magnesium stearate. In some embodiments, the gastric residence system further comprises a time-dependent disintegrating matrix comprising about 44.95 wt % of polycaprolactone (PCL), such as PCL having a viscosity midpoint between about 1.5 dl/g to about 2.1 dl/g, such as Corbion PC17. In some embodiments, the time-dependent disintegrating matrix further comprises about 35.0 wt % of an acid terminated copolymer of DL-lactide and glycolide (50/50 molar ratio) having a viscosity midpoint between about 0.32 dl/g to about 0.48 dl/g (such as about 0.4 dl/g), such as PDLG 5004A. In some embodiments, the time-dependent disintegrating matrix further comprises about 18.0 wt % of a copolymer of DL-lactide and glycolide (50/50 molar ratio) having a viscosity midpoint between about 0.32 dl/g to about 0.48 dl/g (such as about 0.4 dl/g), such as PDLG 5004. In some embodiments, the time-dependent disintegrating matrix further comprises about 2.0 wt % of polyethylene glycol, such as polyethylene glycol with average molecular weight of 100,000, such as PEO_{100K}. In some embodiments, the time-dependent disintegrating matrix further comprises about 0.05 wt % of iron oxide, such as E172. In some embodiments, the gastric residence system further comprises a pH-dependent disintegrating matrix comprising about 33.95 wt % of polycaprolactone (PCL), such as PCL having a viscosity midpoint between about 1.5 dl/g to about 2.1 dl/g, such as Corbion PC17. In some embodiments, the pH-dependent disintegrating matrix further comprises about 63.95 wt % of hypromellose acetate succinate, such as HPMCAS-MG. In some embodiments, the pH-dependent disintegrating matrix further comprises about 2.0 wt % of poly(ethylene glycol)-block-poly(propylene glycol)-block-poly(ethylene glycol) polymers, such as H—(OCH2CH2)x-(O—CH(CH3)CH2)y-(OCH2CH2)z-OH where x and z are about 101 and y is about 56, such as Poloxamer 407 (P407). In some embodiments, the pH-dependent disintegrating matrix further comprises about 0.1 wt % of iron oxide, such as E172. In some embodiments, the gastric residence system further comprises one or more inactive segments. In some embodiments, the gastric residence system further comprises a radiopaque segment comprising about 70 wt % of polycaprolactone (PCL), such as PCL having a viscosity midpoint between about 1.5 dl/g to about 2.1 dl/g, such as Corbion PC17. In some embodiments, the radiopaque segment comprises about 30 wt % of (BiO)₂CO₃. In some embodiments, the gastric residence system further comprises one or more polymeric linker segments comprising about 100 wt % of polycaprolactone (PCL). In some embodiments, a polymeric linker segment is located at the proximal end of the arm, immediately adjacent to the central elastomer.

In some embodiments, the gastric residence system has one arm comprising a drug-eluting segment and five arms not comprising a drug-eluting segment. In some embodiments, the gastric residence system has two arms comprising a drug-eluting segment and four arms not comprising a drug-eluting segment. In some embodiments, the gastric residence system has three arms comprising a drug-eluting segment and three arms not comprising a drug eluting segment. In some embodiments, the gastric residence system has four arms comprising a drug-eluting segment and two arms not comprising a drug-eluting segment. In some embodiment, the gastric residence system has five arms comprising a drug-eluting segment and one arm not comprising a drug-eluting segment. In some embodiments, the gastric residence system has six arms comprising a drug-eluting segment.

### Central Elastomer

The central elastomer provides the gastric residence system with the ability to be compacted into a compressed configuration, which can be placed in a capsule or other suitable containing structure for administration to a subject.

In some embodiments, a dosage form for administration of one or more agents comprises a gastric residence system, wherein the gastric residence system comprises a central elastomer comprising a liquid silicone rubber (LSR). In some embodiments, the LSR has a hardness of about 45 to about 60 durometer.

In some embodiments, a dosage form for administration of one or more agents comprises a gastric residence system, wherein the gastric residence system comprises a central elastomer comprising a liquid silicone rubber (LSR). In some embodiments, the LSR has a hardness of about 45 to about 55 durometer.

In some embodiments, a dosage form for administration of one or more agents comprises a gastric residence system, wherein the gastric residence system comprises a central elastomer comprising a liquid silicone rubber (LSR). In some embodiments, the LSR has a hardness of about 50 durometer.

### EXAMPLES

The disclosure is further illustrated by the following non-limiting examples.

### Example 1: Buprenorphine-Naloxone Dosage Form (Extended-Release Gastric Residence System)

In this Example, a dosage form according to the present invention includes a gastric residence system, the gastric residence system is formulated to include buprenorphine and naloxone.

The gastric residence system includes a central elastomer that provides the gastric residence system with the ability to be compacted into a compressed configuration. The gastric residence system illustrated in this Example is another different arrangement of the “star” configuration. In an example of the buprenorphine-naloxone-formulated gastric residence system, the stellate contains 6 arms each comprising a drug-eluting segment.

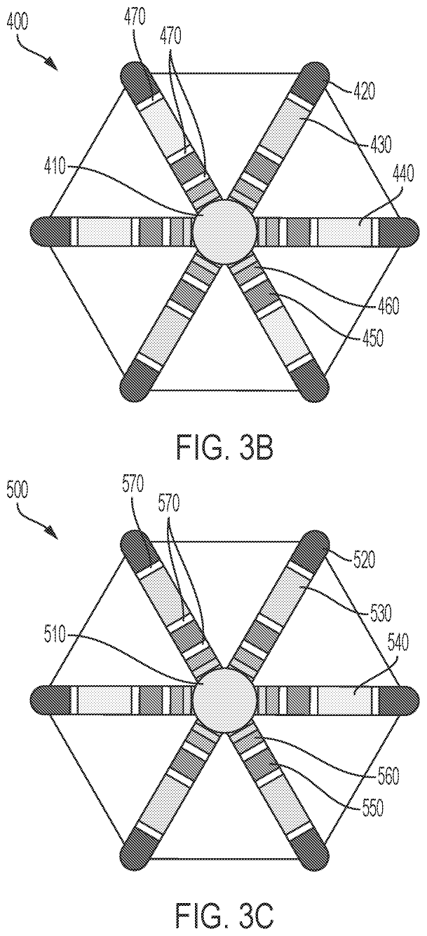

FIG. 3A is labelled to show the various elements of this configuration. The system 300 comprises a central elastomeric core 310 which is in the shape of an “asterisk” having six short branches. A segment 380 of the arm is attached to one short asterisk branch. The segment 380 is followed by a segments 360, a first segment 370, a segment 350, a second segment 370, a segment 340, and a third segment 370 in sequence. The distal end of each arm has segment 320.

The gastric residence system has an average size of about 46 mm and each segment has a length ranging from about 0.5 mm to about 8.4 mm. The table below provides a listing of the length of each segment of an active arm (i.e., an arm comprising a drug-eluting segment) in the gastric residence system. Each range or value below can be considered to be “about” the range or value indicated, or exactly the range or value indicated.

| Segment | Length | |
|---|---|---|
| 320 | 4 | mm |
| 340 | 8.4 | mm |
| 350 | 1.85 | mm |
| 360 | 1.0 | mm |
| 370 | 0.5 | mm |
| 380 | 1-1.5 | mm |

The central elastomeric core 310 comprises a liquid silicone rubber (LSR) having a hardness of 50 durometer.

In this example, the dosage form provided here contains 6 arms each comprising a drug-eluting segment, wherein the dosage form comprises about 115 mg of buprenorphine and about 10 mg naloxone for administration. Buprenorphine and naloxone are included in a carrier polymer-agent segment 340 (e.g., a drug-eluting segment). The drug-eluting segment comprises about 45 wt % of buprenorphine, about 4.5 wt % naloxone, about 39.5 wt % of Corbion PC17, about 3.0 wt % of P407, about 7 wt % PEO_{100K}, about 0.5 wt % of Vitamin E succinate, and about 0.5 wt % of SiO₂. Also contemplated in the present application are variations of this dosage form with increased numbers and/or lengths of the drug-eluting segments to achieve higher doses of the drug, for example, buprenorphine and/or naloxone.

The gastric residence system further includes a time-dependent disintegrating matrix or linker, referred as the segment 360, as well as a pH-dependent disintegrating matrix or linker, referred as the segment 350. In addition, the gastric residence system includes a structural segment 370, and a polymeric linker segment 380.

The time-dependent disintegrating matrix (segment 360) comprises about 44.95 wt % Corbion PC17, about 35 wt % of acid terminated copolymer of DL-lactide and glycolide (PDLG5004A), about 18 wt % of copolymer of DL-lactide and glycolide (PDLG5004), about 2 wt % of polyethylene glycol 100 k and about 0.05 wt % color-absorbing dye E172. The pH-dependent disintegrating matrix (segment 350) comprises about 63.95 wt % HPMCAS, about 33.95 wt % Corbion PC17, about 2 wt % P407 and about 0.1 wt % color-absorbing dye E172. The structural segment 370 can be a radiopaque-PCL segment, comprising about 70 wt % PCL, and about 30 wt % (BiO)₂CO₃.

Segment 320, at the distal end of each arm, is a third disintegrating matrix, to which a filament is also optionally attached, where the filament thereby circumferentially connects the arms. The third disintegrating matrix (Segment 320) comprises about 64.9 wt % HPMCAS, about 30 wt % PCL, about 2.5 wt % propylene glycol, about 2.5 wt % stearic acid, and about 0.1 wt % iron oxide (for example about 0.025% ferrosoferric oxide and about 0.075% FD&C Red 40).

Segment 380, at the proximal end of each arm, is a polymeric linker segment comprising about 100 wt % PCL.

In some embodiments, each drug arm is coated by a release rate-modulating film. Specifically, the coating comprises about 73.5 wt % of Corbion PC17, about 24.5 wt % of VA64 and about 2% of Magnesium Stearate, and is applied in an amount of about 1% of the pre-coating weight of the segment.

The gastric residence system is assembled and then placed into an appropriate sized capsule as described in Example 1 of International Patent Application PCT/US2020/059541. The dosage form described here differs from a gastric residence system previously described in International Patent Application No. PCT/US2020/059541, and other gastric residence systems previously designated as LYN-005.

In another example of the buprenorphine-naloxone-formulated gastric residence system, the stellate contains 4 arms each comprising a drug-eluting segment, and 2 arms not comprising a drug-eluting segment. Also contemplated in this application are other gastric residence systems containing 6 arms of which either 1, 2, 3, 4, 5, to 6 arms comprise a drug-eluting segment.

FIG. 3B is labelled to show the various elements of this configuration. The system 400 comprises a central elastomeric core 410 which is in the shape of an “asterisk” having six short branches.

For an arm containing a drug-eluting segment, a segment 480 of the arm is attached to one short asterisk branch. The segment 480 is followed by a segment 460, a first segment 470, a segment 450, a second segment 470, a segment 440, and a third segment 470 in sequence. The distal end of each drug-containing arm has segment 420.

For an arm not containing a drug-eluting segment, a segment 480 of the arm is attached to one short asterisk branch. The segment 480 is followed by a segment 460, a first segment 470, a segment 450, a second segment 470, a segment 430, and a third segment 470. The distal end of each drug-free arm has segment 420.

The gastric residence system has an average size of about 46 mm and each segment has a length ranging from about 0.5 mm to about 8.4 mm. The table below provides a listing of the length of each segment in the gastric residence system. Each range or value below can be considered to be “about” the range or value indicated, or exactly the range or value indicated.

| Segment | Length | |
|---|---|---|
| 420 | 4 | mm |
| 430 | 8.4 | mm |
| 440 | 8.4 | mm |
| 450 | 1.85 | mm |
| 460 | 1.0 | mm |
| 470 | 0.5 | mm |
| 480 | 1-1.5 | mm |

The central elastomeric core 410 comprises a liquid silicone rubber (LSR) having a hardness of 50 durometer.

In this example, the dosage form provided here contains 6 arms, four of which comprise a drug-eluting segment, wherein the dosage form comprises about 80 mg of buprenorphine and about 7 mg naloxone for administration. Buprenorphine and naloxone are included in a carrier polymer-agent segment 440 (e.g., a drug-eluting segment). The drug-eluting segment comprises about 45 wt % of buprenorphine, about 4.5 mg naloxone, about 39.5 wt % of Corbion PC17, about 7 wt % PEO100K, about 3 wt % of P407, about 0.5 wt % of Vitamin E succinate, and about 0.5 wt % of SiO₂. Also contemplated in the present application are variations of this dosage form with increased numbers and/or lengths of the drug-eluting segments to achieve higher doses of the drug, for example, buprenorphine and/or naloxone.

The two arms that are drug-free comprise an inactive segment (segment 430). The inactive segment 430 each comprises about 66.45 wt % of Corbion PC17, about 32.0 wt % of VA 64, about 1.5 wt % of P407 and about 0.05 wt % of FD&C Blue 1 Aluminum lake.

The gastric residence system further includes a time-dependent disintegrating matrix or linker, referred as the segment 460, as well as a pH-dependent disintegrating matrix or linker, referred as the segment 450. In addition, the gastric residence system includes a structural segment 470.

The time-dependent disintegrating matrix (segment 460) comprises about 44.95 wt % Corbion PC17, about 35 wt % of acid terminated copolymer of DL-lactide and glycolide (PDLG5004A), about 18 wt % of copolymer of DL-lactide and glycolide (PDLG5004), about 2 wt % of polyethylene glycol 100 k and about 0.05 wt % color-absorbing dye E172. The pH-dependent disintegrating matrix (segment 450) comprises about 63.95 wt % HPMCAS, about 33.95 wt % Corbion PC17, about 2 wt % P407 and about 0.1 wt % color-absorbing dye E172. The structural segment 470 can be a radiopaque-PCL segment, comprising about 70 wt % PCL, and about 30 wt % (BiO)₂CO₃.

Segment 420, at the distal end of each arm, is a third disintegrating matrix, to which a filament is also optionally attached, where the filament thereby circumferentially connects the arms. The third disintegrating matrix (Segment 420) comprises about 64.9 wt % HPMCAS, about 30 wt % PCL, about 2.5 wt % propylene glycol, about 2.5 wt % stearic acid, and about 0.1 wt % iron oxide (for example about 0.025% ferrosoferric oxide and about 0.075% FD&C Red 40).

Segment 480, at the proximal end of each arm, is a polymeric linker segment comprising about 100 wt % PCL.

In some embodiments, each drug arm is coated by a release rate-modulating film. Specifically, the coating comprises about 73.5 wt % of Corbion PC17, about 24.5 wt % of VA64 and about 2% of Magnesium Stearate, and is applied in an amount of about 1% of the pre-coating weight of the segment.

The gastric residence system is assembled and then placed into an appropriate sized capsule as described in Example 1 of International Patent Application PCT/US2020/059541. The dosage form described here differs from a gastric residence system previously described in International Patent Application No. PCT/US2020/059541, and other gastric residence systems previously designated as LYN-005.

In another example of the buprenorphine-naloxone-formulated gastric residence system, the stellate contains 2 arms each comprising a drug-eluting segment, and 4 arms not comprising a drug-eluting segment. Also contemplated in this application are other gastric residence systems containing 6 arms of which either 1, 2, 3, 4, 5, to 6 arms comprise a drug-eluting segment.

FIG. 3C is labelled to show the various elements of this configuration. The system 500 comprises a central elastomeric core 510 which is in the shape of an “asterisk” having six short branches.

For an arm containing a drug-eluting segment, a segment 580 of the arm is attached to one short asterisk branch. The segment 580 is followed by a segment 560, a first segment 570, a segment 550, a second segment 570, a segment 540, and a third segment 570 in sequence. The distal end of each drug-containing arm has segment 520.

For an arm not containing a drug-eluting segment, a segment 580 of the arm is attached to one short asterisk branch. The segment 580 is followed by a segment 560, a first segment 570, a segment 550, a second segment 570, a segment 530, and a third segment 570. The distal end of each drug-free arm has segment 520.

The gastric residence system has an average size of about 46 mm and each segment has a length ranging from about 0.5 mm to about 8.4 mm. The table below provides a listing of the length of each segment in the gastric residence system. Each range or value below can be considered to be “about” the range or value indicated, or exactly the range or value indicated.

| Segment | Length | |
|---|---|---|
| 520 | 4 | mm |
| 530 | 8.4 | mm |
| 540 | 8.4 | mm |
| 550 | 1.85 | mm |
| 560 | 1.0 | mm |
| 570 | 0.5 | mm |
| 580 | 1-1.5 | mm |

The central elastomeric core 510 comprises a liquid silicone rubber (LSR) having a hardness of 50 durometer.

In this example, the dosage form provided here contains 6 arms, two of which comprise a drug-eluting segment, wherein the dosage form comprises about 40 mg of buprenorphine and about 3.5 mg naloxone for administration. Buprenorphine and naloxone are included in a carrier polymer-agent segment 540 (e.g., a drug-eluting segment). The drug-eluting segment comprises about 45 wt % of buprenorphine, about 4.5 mg naloxone, about 39.5 wt % of Corbion PC17, about 7 wt % PEO100K, about 3 wt % of P407, about 0.5 wt % of Vitamin E succinate, and about 0.5 wt % of SiO₂. Also contemplated in the present application are variations of this dosage form with increased numbers and/or lengths of the drug-eluting segments to achieve higher doses of the drug, for example, buprenorphine and/or naloxone.

The two arms that are drug-free comprise an inactive segment (segment 530). The inactive segment 530 each comprises about 66.45 wt % of Corbion PC17, about 32.0 wt % of VA 64, about 1.5 wt % of P407 and about 0.05 wt % of FD&C Blue 1 Aluminum lake.

The gastric residence system further includes a time-dependent disintegrating matrix or linker, referred as the segment 560, as well as a pH-dependent disintegrating matrix or linker, referred as the segment 550. In addition, the gastric residence system includes a structural segment 570.

The time-dependent disintegrating matrix (segment 560) comprises about 44.95 wt % Corbion PC17, about 35 wt % of acid terminated copolymer of DL-lactide and glycolide (PDLG5004A), about 18 wt % of copolymer of DL-lactide and glycolide (PDLG5004), about 2 wt % of polyethylene glycol 100 k and about 0.05 wt % color-absorbing dye E172. The pH-dependent disintegrating matrix (segment 550) comprises about 63.95 wt % HPMCAS, about 33.95 wt % Corbion PC17, about 2 wt % P407 and about 0.1 wt % color-absorbing dye E172. The structural segment 570 can be a radiopaque-PCL segment, comprising about 70 wt % PCL, and about 30 wt % (BiO)₂CO₃.

Segment 520, at the distal end of each arm, is a third disintegrating matrix, to which a filament is also optionally attached, where the filament thereby circumferentially connects the arms. The third disintegrating matrix (Segment 520) comprises about 64.9 wt % HPMCAS, about 30 wt % PCL, about 2.5 wt % propylene glycol, about 2.5 wt % stearic acid, and about 0.1 wt % iron oxide (for example about 0.025% ferrosoferric oxide and about 0.075% FD&C Red 40).

Segment 580, at the proximal end of each arm, is a polymeric linker segment comprising about 100 wt % PCL.

In some embodiments, each drug arm is coated by a release rate-modulating film. Specifically, the coating comprises about 73.5 wt % of Corbion PC17, about 24.5 wt % of VA64 and about 2% of Magnesium Stearate, and is applied in an amount of about 1% of the pre-coating weight of the segment.

The gastric residence system is assembled and then placed into an appropriate sized capsule as described in Example 1 of International Patent Application PCT/US2020/059541. The dosage form described here differs from a gastric residence system previously described in International Patent Application No. PCT/US2020/059541, and other gastric residence systems previously designated as LYN-005.

### Example 2: Evaluating BUP Release for Coated and Uncoated Dosage Forms

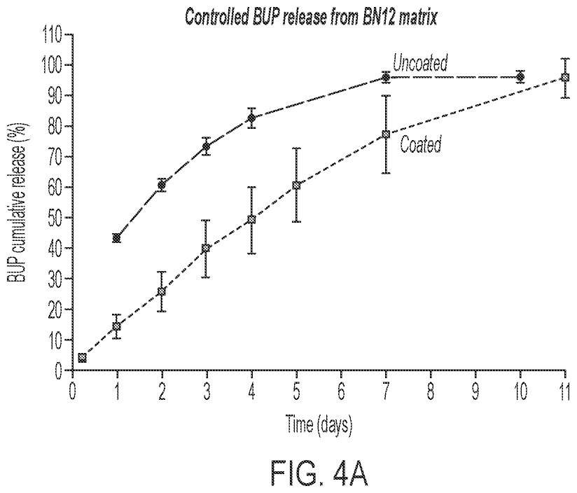

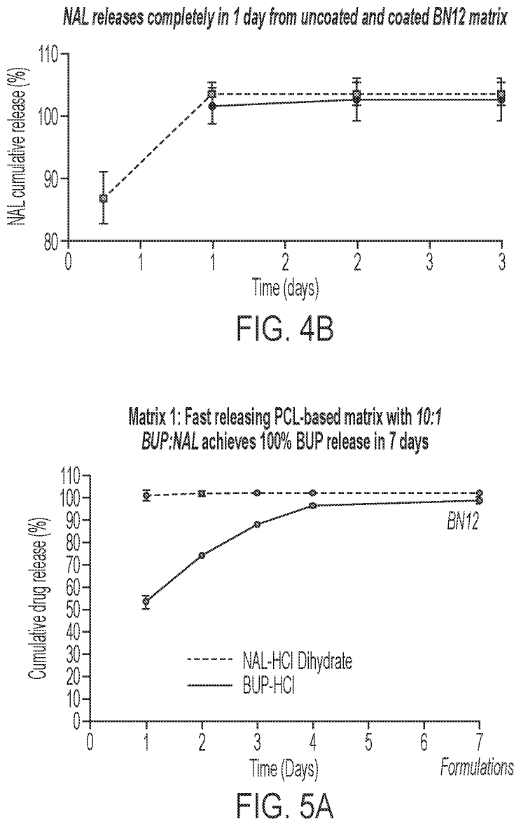

FIGS. 4A and 4B show a BUP release profile and a NAL release profile, respectively, for a dosage form comprising drug-eluting segments comprising a buprenorphine-naloxone combination formulation. Specifically, the buprenorphine-naloxone combination formulation used in the dosage forms is as follows:

| Component | Wt. % |
|---|---|
| BUP | 45 |
| NAL | 4.5 |
| PCL | 39.5 |
| P407 | 3 |
| PEO100K | 7 |
| Vitamin E | 0.5 |
| Silica | 0.5 |

The gastric residence dosage form used for this trial comprised 6 arms, each of them comprising drug-eluting segments. Each dosage form comprised 170 mg BUP. The dosage forms comprised a central elastomer of 50 A silicone. Each arm comprised a polymeric linker segment, time-dependent disintegrating matrix, an enteric disintegrating matrix, a radiopaque polycaprolactone segment, an inactive segment, a drug-eluting segment, and a radioactive polycaprolactone segment, in that order (wherein the polymeric linker segment was coupled to the central elastomer). The total diameter of the uncompacted dosage form was 46 mm. The drug-eluting segments were fabricated using Haake mini-CTW conical twin screw compounder and compression molded.

The dosage forms were coated with a 1% PCL:VA64 75:25 coating. As shown in FIG. 3A, the coated dosage forms can control burst release and linearize the BUP release from the combination formulation of the drug-eluting segments. FIG. 3B shows the NAL completely releases from the combination formulation after one day.

### Example 3: Comparing Release Profiles of Buprenorphine-Naloxone Combination Formulation with Naloxone-Only Formulation

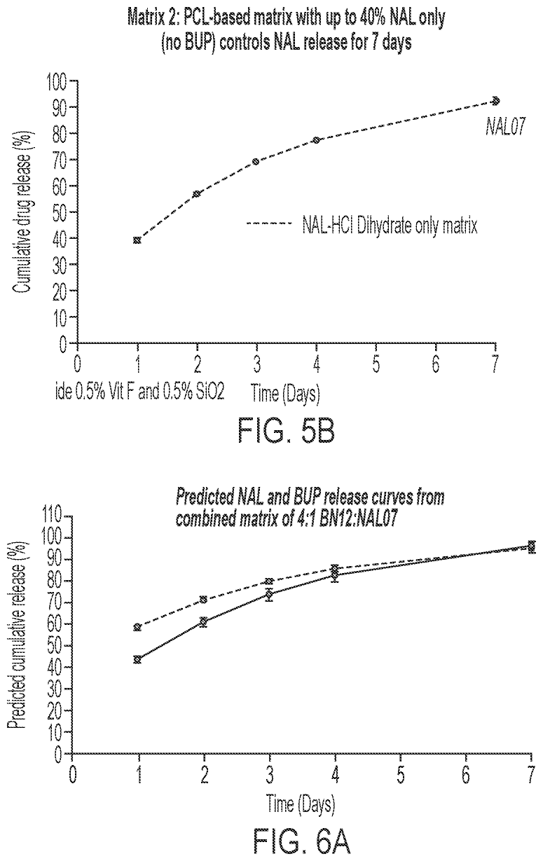

FIGS. 5A and 5B show release profiles for a buprenorphine-naloxone combination formulation (FIG. 5A) compared to a naloxone-only formulation (FIG. 5B). As shown, FIG. 5A shows the release profiles of the BUP and NAL releasing from the buprenorphine-naloxone combination formulation. The NAL completely releases after only one day. The BUP completely releases in seven days. FIG. 5B shows that from a naloxone-only formulation, the naloxone almost completely releases after 7 days. The formulations of the buprenorphine-naloxone combination formulation and the naloxone-only formulation are as follows:

| Component | Wt. % |
|---|---|
| BUP | 45 |
| NAL | 4.5 |
| PCL | 39.5 |
| P407 | 3 |
| PEO100K | 7 |
| Vitamin E | 0.5 |
| Silica | 0.5 |
| NAL | 40 |
| PCL | 56 |
| P407 | 3 |
| Vitamin E | 0.5 |
| Silica | 0.5 |

### Example 4: Co-Extruded Drug-Eluting Segments with Buprenorphine-Naloxone Combination Formulation and Naloxone-Only Formulation

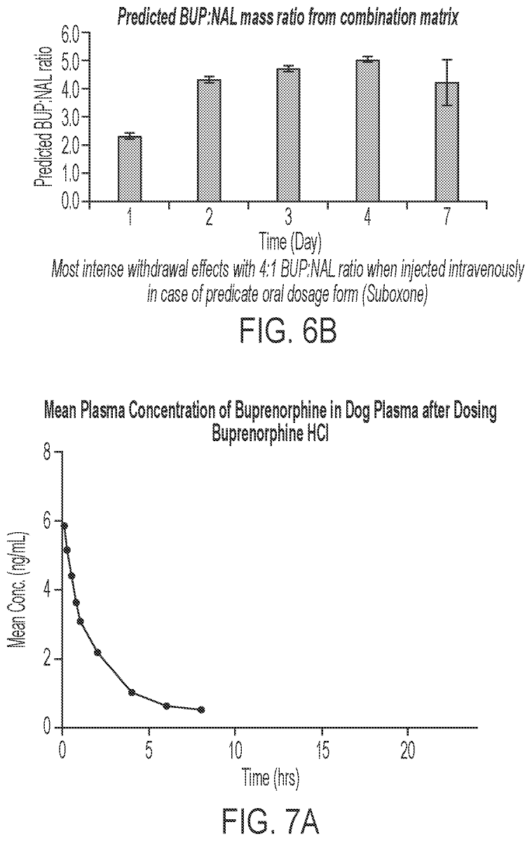

As described herein, some gastric residence dosage forms comprise drug-eluting segments co-extruded with a buprenorphine-naloxone combination formulation and a naloxone-only formulation. FIGS. 6A and 6B show release profiles of BUP and NAL from a co-extruded drug-eluting segment, respectively, and mass ratios from a co-extruded associated drug-eluting segment over the course of the first seven days after ingestion.

Specifically, FIG. 6A shows predicted release profiles for a co-extruded drug eluting segment comprising a buprenorphine-naloxone combination formulation and a naloxone-only formulation co-extruded in a 4:1 ratio. The dotted line represents predicted naloxone release from a co-extruded drug eluting segment, and the solid line shows the predicted buprenorphine release from a co-extruded drug eluting segment. The buprenorphine-naloxone formulation and the naloxone-only formulation are the same as those provided in Example 3, above. As shown, the co-extruded drug-eluting segment achieves similar release profiles for both buprenorphine and naloxone over a 7 day residence period.

FIG. 6B shows that the predicted release rates of buprenorphine and naloxone from a co-extruded drug-eluting segment, as shown in FIG. 6A, would achieve a buprenorphine and naloxone mass ratio shown in 6B. (In case of predicate oral dosage form (Suboxone) the most intense withdrawal effects were observed with 4:1 BUP:NAL mass ratio when injected intravenously).

### Example 5: Study of Drug Release in Beagles

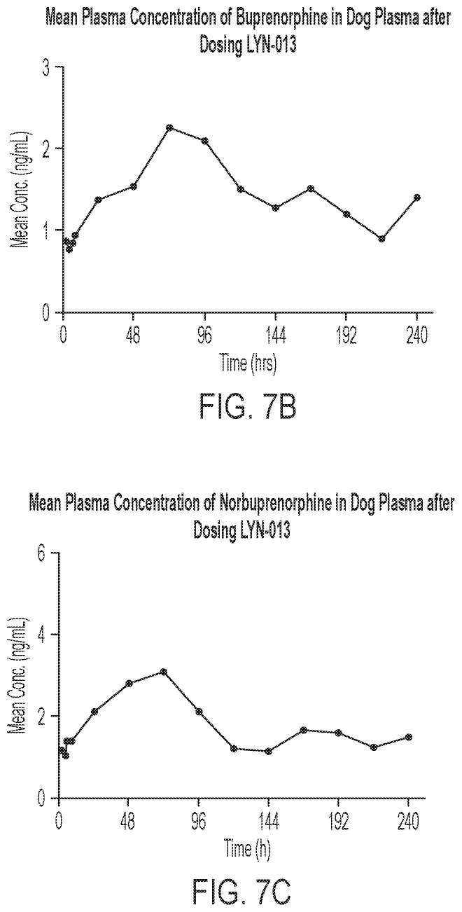

The release of BUP and NAL when administered sublingually was compared to the release of BUP and NAL when administered using a gastric residence dosage form as described herein. The results are provided in FIGS. 7A-7C.

A total of four female beagle's (approximately 7-12 kg at time of dosing) were selected from the Testing Facility's colony of sponsor-designated animals. All animals were fasted overnight prior to both Dose 1 and Dose 2. Food was returned to animals at least 1 hour post-recovery from anesthesia following Dose 1. For Dose 2, animals were given a small handful of canine chow ˜30 (±5) minutes prior to dose administration, and food was returned to animals 1 hour post-dose. The final study design is presented in the table below.

| | Number of | | | Dose* | | Post- |
|---|---|---|---|---|---|---|
| | Female | | | (mg/ | | Dose |
| Group | Beagles | Dose | Test Article | animal) | Route | Chase |
| 1 | 4 | 1 | Buprenorphine | 1 mL | Sublin- | N/A |
| | | | HCL | | gual | |
| | | 2 | LYN-013 | 1 | Oral | Wet |
| | | | (gastric | capsule | | food |
| | | | residence | | | chase |
| | | | dosage form) | | | |

*Represents the nominal amount of test material administered and the maximum potential total exposure for each animal; extended release dose was anticipated to be 2.4 mg/kg/day buprenorphine and 0.2 mg/kg/day naloxone for 7 days.

On Day 1, each animal was sedated with Dexmedetomidine (16.8 mcg/kg, IV). Following sedation, each animal's head was positioned and Dose 1 (Buprenorphine HCL) was administered into the sublingual space for an exposure period of 10 minutes. After the exposure period, Dose 1 was rinsed and wiped from the sublingual space and each animal received Antisedan (200 mcg/kg, IM) for reversal of the sedation. The Dose 2 Test Article, LYN-013, was received from the Sponsor in ready-to-use form. LYN-013 comprises a 50 A central elastomer with an enteric disintegrating matrix, a time-dependent disintegrating, and 6×BUP/Nal drug arms, with a final diameter of ˜46 mm (consistent with the dosage form described above with respect to FIG. 3A, and in the table below).

| | | | | | | |
|---|---|---|---|---|---|---|
| | | | Folding | Axial | | Drug |
| | | Dosage | Force | Stiff- | Diameter | Dose |
| Group | Dog # | Form # | (N)* | ness* | (mm) | (mg) |

| | | | | |
|---|---|---|---|---|
| 1 | 1509 | 211-02-01 | 46 | 171 BUP/ |
| (Dose 2) | 1510 | 211-02-02 | | 15.5 Nal |
| | 1511 | 211-02-03 | | |
| | 1512 | 211-02-04 | | |

*Folding force and axial stiffness were not evaluated.
Note:
“NB” and “PG” were missing from Lot numbers on individual capsule packages, so Lot numbers appeared as 211-02-0X instead of NB211-PG02-0X. Both versions were correct and equivalent.
Individual capsules were labeled as 1-4.

Each animal received the capsule dose, containing the dosage form, as detailed in the table above by manual oral administration on Day 16. Immediately after dosing, each animal was provided with a small amount of canned food loosely packed into a ball, approximately 1 inch in size, to assist in swallowing the capsule.

All dosing was completed without incident. Dose administration data including animal body weights are presented in the table below.

| | | | | | | | | Animal | Formulation |
|---|---|---|---|---|---|---|---|---|---|
| Dosing | Test | Group | Animal | Dosing | | USDA | Dose | Weight | Administered |
| Day | Article | No. | No. | Date | Sex | No. | Route | (kg) | (mg/animal^{a}) |
| 1 | Buprenorphine | 1 | 1509 | 14 Aug. | F | 3745695 | Sublingual | 8.6 | 0.3 |
| | HCL^{b} | | | 2019 | | | Infusion | | |
| 1 | Buprenorphine | 1 | 1510 | 14 Aug. | F | 3754546 | Sublingual | 9.3 | 0.3 |
| | HCL^{b} | | | 2019 | | | Infusion | | |
| 1 | Buprenorphine | 1 | 1511 | 14 Aug. | F | 3739912 | Sublingual | 8.5 | 0.3 |
| | HCL^{b} | | | 2019 | | | Infusion | | |
| 1 | Buprenorphine | 1 | 1512 | 14 Aug. | F | 3750249 | Sublingual | 8.6 | 0.3 |
| | HCL^{b} | | | 2019 | | | Infusion | | |
| 16 | LYN-013 | 1 | 1509 | 29 Aug. | F | 3745695 | Oral | 8.8 | 171 BUP/ |
| | | | | 2019 | | | Capsule | | 15.5 Nal |
| 16 | LYN-013 | 1 | 1510 | 29 Aug. | F | 3754546 | Oral | 8 | 171 BUP/ |
| | | | | 2019 | | | Capsule | | 15.5 Nal |
| 16 | LYN-013 | 1 | 1511 | 29 Aug. | F | 3739912 | Oral | 8.7 | 171 BUP/ |
| | | | | 2019 | | | Capsule | | 15.5 Nal |
| 16 | LYN-013 | 1 | 1512 | 29 Aug. | F | 3750249 | Oral | 8.6 | 171 BUP/ |
| | | | | 2019 | | | Capsule | | 15.5 Nal |

^{a}Nominal amount.
^{b}Prior to Dose 1, all animals received Dexmedetomidine (16.8 mcg/kg. IV) for sedation. Post-Dose 1, all animals received Antisedan (200 mcg/kg. IM) for reversal of the sedation.

X-rays showed the stomach and intestines to determine the positioning of the dosage form within the gastrointestinal system. Radiographs were performed 30 minutes following dose administration on Day 16 in order to confirm capsule dissolution. Additionally, x-rays were taken daily through Day 8, on Day 10, and then 3 times weekly, or as appropriate, until the dosage form, in its entirety, had passed through the body as confirmed by imaging.

All blood samples were collected by venipuncture of a peripheral vein. A volume of 1 mL of whole blood was collected into tubes containing K3EDTA anticoagulant at each time point. Whole blood samples with K3EDTA were kept on wet ice before processing for plasma. Samples were centrifuged at 2200×g for 10 minutes at 5° C.±3° C. to isolate plasma. Plasma samples were transferred to individual polypropylene tubes in a 96-well plate format and immediately placed on dry ice until storage at nominally −80° C. prior to shipment. The actual blood collection times are presented below.

| Group | | |
|---|---|---|
| Numbers/Collection | | |
| Information | Study Day | Whole Blood for Plasma |
| 1 | 1 (Dose 1) | Pre-dose, 5, 15, 30, 45 minutes (0.083, 0.25, 0.5, and |
| | | 0.75 hours, respectively) |
| | | and 1, 2, 4, 6, 8 12, 24 hours post the end of dosing |
| 1 | 16 (Dose 2) | Pre = dose, 2, 4, 6, 8, 24, 48, 72, 96, 120, 144, 168, |
| | | 192, 216 and 240 hours post-dose* |

| | |
|---|---|
| Anti-Coagulant | K₃EDTA |
| Volume/Time Point | 1 mL of whole blood |

*Animals that had confirmed passage of dose form by radiographs before study completion, had continued blood collection for an additional 48 hours only.

Collected samples were shipped to Charles River Laboratories, Worcester, MA, by same-day courier. Plasma samples were analyzed for concentrations of buprenorphine, norbuprenorphine, and naloxone (buprenorphine-3-glucuronide and norbuprenorphine-3-glucuronide was monitored), using an appropriate analytical procedure by LCMS.

FIG. 7A shows the mean plasma concentrations of BUP in dog plasma after dosing BUP-HCL sublingually. FIG. 7B shows the mean plasma concentrations of BUP in dog plasma after dosing BUP in a gastric residence dosage form (LYN-013). FIG. 7C shows mean plasma concentrations of norbuprenorphine (a major active metabolite of buprenorphine) after dosing BUP in a gastric residence dosage form (LYN-013). This study demonstrates prolonged BUP plasma levels of greater than approximately 1 ng/mL for more than one week after administration of a single oral capsule.

### Example 6: Study of BUP/NAL Release in Cynomolgus Monkeys

This report includes two single dose pharmacokinetic studies (LYN-190-BUP, LYN-213-BUP).

In each study, monkeys were fasted overnight prior to dose administration. In study LYN-190-BUP, a 0.03 mg/kg intravenous bolus dose of Buprenorphine HCL was administered to 4 monkeys and plasma samples were collected at 0.083, 0.25, 0.5, 1, 2, 4, 6, 8 and 24 hours post-dose. In study LYN-213-BUP, a 172/17.2 mg buprenorphine/naloxone capsule of LYN-013 was orally administered to 4 monkeys and plasma samples were collected at pre-dose, 2, 4, 6, 8, 10, 12, 24, 48, 72, 96, 120, 144 and 216 hours post-dose.

For each study, sample extracts were prepared by protein precipitation from plasma, and buprenorphine, norbuprenorphine (active metabolite), and naloxone (abuse deterrent) concentrations were measured using LC/MS/MS. Pharmacokinetic parameters of buprenorphine and norbuprenorphine (where possible), were calculated by noncompartmental analysis.

Following an intravenous dose of 0.03 mg/kg Buprenorphine HCL to monkeys, the extrapolated initial plasma concentration (CO) was 17.3 ng/mL. The area under the concentration-time curve from time zero to the time of the last sample (AUClast) was 24.7 hr*ng/mL and the terminal phase half-life (t½) was 1.62 hr.

After a single 172/17.2 mg buprenorphine/naloxone dose, LYN-013 had measurable plasma buprenorphine concentrations for up to 9 days post-dose while levels of norbuprenorphine were measurable through 5 days and naloxone was bq1 in all samples (with the exception of 1 sample). Buprenorphine had a median 72 hr Tmax and a Cmax of 4.85 ng/mL. The AUClast of buprenorphine was 350 hr*ng/ml with a t½ of 26.4 hr. The absolute oral bioavailability (F) was 1.1%. Norbuprenorphine, reached Tmax at 60 hr and had a lower Cmax and AUClast (2.72 ng/ml and 151 hr*ng/ml, respectively).

The Table below shows the individual and mean plasma PK parameters for iv BUP-HCL and LYN-013 (buprenorphine and norbuprenorphine).

| | | | | | | | Half- | | | | | |
|---|---|---|---|---|---|---|---|---|---|---|---|---|
| | | | | Tmax\hat{ } | Tlast\hat{ } | Cmax† | life | AUClast | AUCinf | AUC24 | AUC168 | F |
| Dose | Analyte | ROA | Animal | (hr) | (hr) | (ng/mL) | (hr) | (hr*ng/mL) | (hr*ng/mL) | (hr*ng/mL) | (hr*ng/mL) | (%) |
| 0.03 | Buprenorphine | iv | 1002 | 0.083 | 8 | 15.6 | 1.82 | 20.9 | 21.5 | — | — | |
| mg/kg | HCL | | 1003 | 0.250 | 8 | 15.0 | 1.47 | 28.0 | 28.7 | — | — | |
| | | | 1004 | 0.083 | 8 | 15.0 | 1.64 | 27.3 | 28.0 | — | — | |
| | | | 1101 | 0.083 | 8 | 23.5 | 1.57 | 22.5 | 23.6 | — | — | |
| | | | Mean | 0.083 | 8 | 17.3 | 1.62 | 24.7 | 25.4 | — | — | |
| | | | SD | — | — | 4.16 | 0.15 | 3.47 | 3.45 | — | — | |
| | | | CV % | — | — | 24.0 | 9.21 | 14.1 | 13.6 | — | — | |
| 172 | Buprenorphine | LYN- | 1001 | 72 | 144 | 4.37 | 23.0 | 277 | 284 | 63.5 | 280 | |
| mg | | 013 | 1002 | 120 | 144 | 5.8 | — | 396 | — | 43.0 | — | |
| | | | 1101 | 72 | 216 | 6.48 | 35.4 | 576 | 590 | 72.5 | 549 | |
| | | | 1102 | 24 | 96 | 2.76 | 20.9 | 151 | 162 | 44.2 | 162 | |
| | | | Mean | 72 | 144 | 4.85 | 26.4 | 350 | 345 | 55.8 | 330 | 1.1 |
| | | | SD | — | — | 1.65 | 7.88 | 181 | 220 | 14.5 | 198 | |
| | | | CV % | — | — | 34 | 29.8 | 51.7 | 63.8 | 26 | 60 | |
| | Norbuprenorphine | LYN- | 1001 | 72 | 72 | 2.77 | — | 126 | — | 28.9 | — | |
| | | 013 | 1002 | 48 | 48 | 1.85 | — | 57 | — | 21.1 | — | |
| | | | 1101 | 72 | 120 | 4.28 | — | 345 | — | 38.3 | — | |
| | | | 1102 | 12 | 48 | 1.98 | — | 74.5 | — | 35.2 | — | |
| | | | Mean | 60 | 60 | 2.72 | — | 151 | — | 30.9 | — | |
| | | | SD | — | — | 1.12 | — | 133 | — | 7.59 | — | |
| | | | CV % | — | — | 41.1 | — | 88.1 | — | 24.6 | — | |

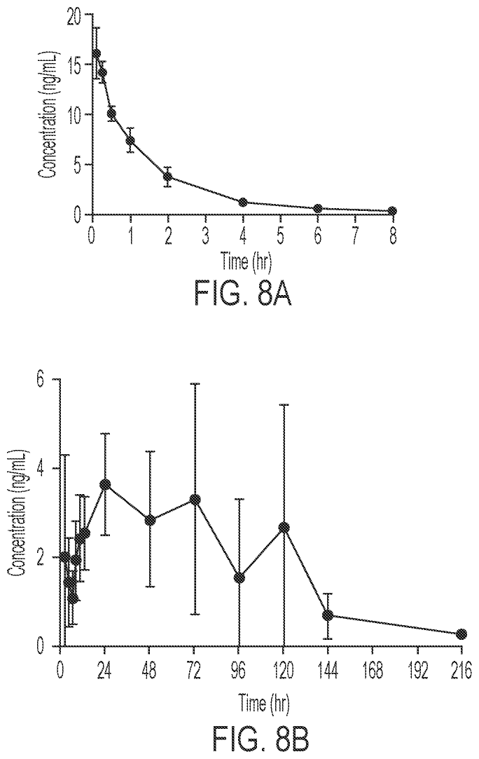

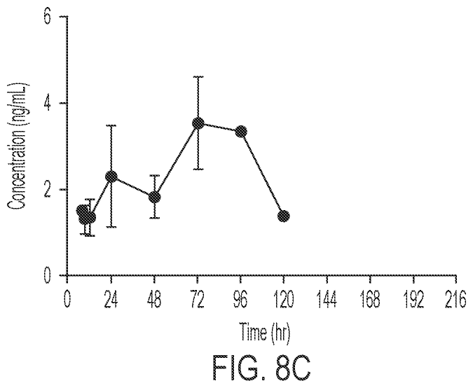

FIGS. 8A-8C show plasma concentrations versus time for a single oral dose of BUP-HCL (iv) (FIG. 8A) and LYN-013 (FIGS. 8B & 8C).

### EMBODIMENTS

Embodiment 1. A gastric residence system comprising: at least one co-extruded drug-eluting component comprising a carrier polymer, buprenorphine or a salt thereof, and naloxone or a salt thereof; and a rate-modulating release film coating the at least one co-extruded drug-eluting component, wherein the gastric residence system is configured to be maintained within a stomach of a human body for at least 48 hours and to release buprenorphine for at least 48 hours, and the at least one co-extruded drug eluting component with the rate-modulating release film is configured to release at least 10% of the buprenorphine or the salt thereof after the first 24 hours of residence within the stomach and at least 10% of the naloxone or the salt thereof after the first 24 hours of residence within the stomach.

Embodiment 2. The gastric residence system of embodiment 1, wherein the rate-modulating release film comprises polycaprolactone and copovidone.

Embodiment 3. The gastric residence system of embodiment 2, wherein the rate-modulating release film comprises 60-90 wt % polycaprolactone.

Embodiment 4. The gastric residence system of embodiment 2 or 3, wherein the rate-modulating release film comprises 10-40 wt % copovidone.

Embodiment 5. The gastric residence system of any of embodiments 1-4, wherein the at least one co-extruded drug-eluting component comprises a first co-extruded portion comprising naloxone and a second co-extruded portion comprising buprenorphine and naloxone.

Embodiment 6. The gastric residence system of embodiment 5, wherein the first co-extruded portion is embedded in the second co-extruded portion.

Embodiment 7. The gastric residence system of embodiment 5 or 6, wherein the first co-extruded portion comprises one or more strands that are embedded within the second co-extruded portion.

Embodiment 8. The gastric residence system of embodiment 5, wherein the first co-extruded portion is layered on the second co-extruded portion.

Embodiment 9. The gastric residence system of any of embodiments 5-8, wherein the first co-extruded portion is present at a ratio of 4:1 to the second co-extruded portion.

Embodiment 10. The gastric residence system of any of embodiments 5-9, wherein the first co-extruded portion comprises 35-50 wt % buprenorphine.

Embodiment 11. The gastric residence system of any of embodiments 5-10, wherein the first co-extruded portion comprises 2-7 wt % naloxone.

Embodiment 12. The gastric residence system of any of embodiments 5-11, wherein the first co-extruded portion comprises 35-50 wt % polycaprolactone.

Embodiment 13. The gastric residence system of any of embodiments 5-12, wherein the first co-extruded portion comprises polyethylene glycol and a poloxamer.

Embodiment 14. The gastric residence system of any of embodiments 5-13, wherein the second co-extruded portion comprises 30-50 wt % naloxone.

Embodiment 15. The gastric residence system of any of embodiments 5-14, wherein the second co-extruded portion comprises 50-60 wt % polycaprolactone.

Embodiment 16. The gastric residence system of any of embodiments 5-15, wherein the second co-extruded portion comprises poloxamer.

Embodiment 17. The gastric residence system of any of embodiments 1-16, wherein the at least one co-extruded drug-eluting component comprises 30 mg to 40 mg of buprenorphine or a salt thereof.

Embodiment 18. The gastric residence system of any of embodiments 1-17, wherein the at least one co-extruded drug-eluting component comprises 8 mg to 15 mg of naloxone or a salt thereof.

Embodiment 19. The gastric residence system of any of embodiments 1-18, wherein the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one co-extruded drug-eluting component.

Embodiment 20. The gastric residence system of embodiment 19, wherein the plurality of arms comprises six arms.

Embodiment 21. The gastric residence system of embodiment 19 or 20, wherein at least two arms of the plurality of arms comprises a co-extruded drug-eluting component of the at least one co-extruded drug-eluting component.

Embodiment 22. The gastric residence system of embodiment 19 or 20, wherein at least three arms of the plurality of arms comprises a co-extruded drug-eluting component of the at least one co-extruded drug-eluting component.

Embodiment 23. The gastric residence system of embodiment 19 or 20, wherein six arms of the plurality of arms comprises a co-extruded drug-eluting component of the at least one co-extruded drug-eluting component.

Embodiment 24. The gastric residence system of any of embodiments 19-23, wherein each arm of the plurality of arms comprises a polymeric linker segment attached to the central elastomer, the polymeric linker segment comprising polycaprolactone.

Embodiment 25. The gastric residence system of embodiment 24, wherein each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

Embodiment 26. The gastric residence system of embodiment 25, wherein each arm of the plurality of arms comprises a first inert segment attached to the first disintegrating matrix segment, the first inert segment comprising polycaprolactone, and (BiO)₂CO₃.

Embodiment 27. The gastric residence system of embodiment 26, wherein each arm of the plurality of arms comprises a second disintegrating matrix segment attached to the first inert segment, the second disintegrating matrix segment comprising polycaprolactone, hydroxypropyl methylcellulose acetate succinate, and a poloxamer.

Embodiment 28. The gastric residence system of embodiment 27, wherein each arm of the plurality of arms comprises a second inert segment attached to the second disintegrating matrix segment, the second inert segment comprising polycaprolactone, and (BiO)₂CO₃.

Embodiment 29. The gastric residence system of embodiment 28, wherein a first arm of the plurality of arms comprises a co-extruded drug-eluting component of the at least one co-extruded drug-eluting component attached to the second inert segment.

Embodiment 30. The gastric residence system of embodiment 28, wherein a second arm of the plurality of arms comprises an inactive segment attached to the second inert segment, the inactive segment comprising polycaprolactone, copovidone, and a poloxamer.

Embodiment 31. The gastric residence system of embodiment 29 or 30, wherein each arm of the plurality of arms comprises a third inert segment attached to one of the co-extruded drug-eluting component or the inactive segment, the third inert segment comprising polycaprolactone, and (BiO)₂CO₃.

Embodiment 32. The gastric residence system of embodiment 31, wherein each arm of the plurality of arms comprises a third disintegrating matrix segment attached to the third inert segment, the third disintegrating matrix comprising polycaprolactone, hydroxypropyl methylcellulose acetate succinate (HPMCAS); stearic acid; and polypropylene glycol.

Embodiment 33. The gastric residence system of embodiment 32, comprising a filament circumferentially connecting each arm, wherein the circumferential filament is attached to the third disintegrating matrix segment of each arm.

Embodiment 34. A method of treating an opioid abuse disorder in an individual, comprising administering the gastric residence system of any one of embodiments 1-33 to the individual.

Embodiment 35. A method of treating pain in an individual, comprising administering the gastric residence system of any one of embodiments 1-33 to the individual.

Embodiment 36. A gastric residence system comprising: at least one drug-eluting component comprising a carrier polymer, buprenorphine or a salt thereof, and naloxone or a salt thereof; and a rate-modulating release film coating the at least one drug-eluting component, wherein the gastric residence system is configured to be maintained within a stomach of a human body for at least 48 hours and to release buprenorphine for at least 48 hours, and the at least one drug-eluting component with the rate-modulating release film is configured to release at least 10% of the buprenorphine or the salt thereof after the first 24 hours of residence within the stomach and at least 10% of the naloxone or the salt thereof within the first 24 hours of residence within the stomach.

Embodiment 37. The gastric residence system of embodiment 36, wherein the rate-modulating release film comprises polycaprolactone and copovidone.

Embodiment 38. The gastric residence system of embodiment 37, wherein the rate-modulating release film comprises 60-90 wt % polycaprolactone.

Embodiment 39. The gastric residence system of embodiment 36 or 37, wherein the rate-modulating release film comprises 10-40 wt % copovidone.

Embodiment 40. The gastric residence system of any of embodiments 36-39, wherein the at least one drug-eluting component comprises 35-50 wt % buprenorphine.

Embodiment 41. The gastric residence system of any of embodiments 36-40, wherein the at least one drug-eluting component comprises 2-7 wt % naloxone.

Embodiment 42. The gastric residence system of any of embodiments 36-41, wherein the at least one drug-eluting component comprises 35-50 wt % polycaprolactone.

Embodiment 43. The gastric residence system of any of embodiments 36-42, wherein the at least one drug-eluting component comprises polyethylene glycol and a poloxamer.

Embodiment 44. The gastric residence system of any of embodiments 36-43, wherein the at least one drug-eluting component comprises 10 mg to 30 mg of buprenorphine or a salt thereof.

Embodiment 45. The gastric residence system of any of embodiments 36-44, wherein the at least one drug-eluting component comprises 1 mg to 3 mg of naloxone or a salt thereof.

Embodiment 46. The gastric residence system of any of embodiments 36-45, wherein the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one drug-eluting component.

Embodiment 47. The gastric residence system of embodiment 46, wherein the plurality of arms comprises six arms.

Embodiment 48. The gastric residence system of embodiment 46 or 47, wherein at least two arms of the plurality of arms comprises a drug-eluting component of the at least one drug-eluting component.

Embodiment 49. The gastric residence system of embodiment 46 or 47, wherein at least three arms of the plurality of arms comprises a drug-eluting component of the at least one drug-eluting component.

Embodiment 50. The gastric residence system of embodiment 46 or 47, wherein six arms of the plurality of arms comprises a drug-eluting component of the at least one drug-eluting component.

Embodiment 51. The gastric residence system of any of embodiments 46-50, wherein each arm of the plurality of arms comprises a polymeric linker segment attached to the central elastomer, the polymeric linker segment comprising polycaprolactone.

Embodiment 52. The gastric residence system of embodiment 51, wherein each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

Embodiment 53. The gastric residence system of embodiment 52, wherein each arm of the plurality of arms comprises a first inert segment attached to the first disintegrating matrix segment, the first inert segment comprising polycaprolactone, and (BiO)₂CO₃.

Embodiment 54. The gastric residence system of embodiment 53, wherein each arm of the plurality of arms comprises a second disintegrating matrix segment attached to the first inert segment, the second disintegrating matrix segment comprising polycaprolactone, hydroxypropyl methylcellulose acetate succinate, and a poloxamer.

Embodiment 55. The gastric residence system of embodiment 54, wherein each arm of the plurality of arms comprises a second inert segment attached to the second disintegrating matrix segment, the second inert segment comprising polycaprolactone, and (BiO)₂CO₃.

Embodiment 56. The gastric residence system of embodiment 55, wherein a first arm of the plurality of arms comprises a drug-eluting component of the at least one drug-eluting component attached to the second inert segment.

Embodiment 57. The gastric residence system of embodiment 55, wherein a second arm of the plurality of arms comprises an inactive segment attached to the second inert segment, the inactive segment comprising polycaprolactone, copovidone, and a poloxamer.

Embodiment 58. The gastric residence system of embodiment 56 or 57, wherein each arm of the plurality of arms comprises a third inert segment attached to one of the drug-eluting component or the inactive segment, the third inert segment comprising polycaprolactone, and (BiO)₂CO₃.

Embodiment 59. The gastric residence system of embodiment 58, wherein each arm of the plurality of arms comprises a third disintegrating matrix segment attached to the third inert segment, the third disintegrating matrix comprising polycaprolactone, hydroxypropyl methylcellulose acetate succinate (HPMCAS); stearic acid; and polypropylene glycol.

Embodiment 60. The gastric residence system of embodiment 59, comprising a filament circumferentially connecting each arm, wherein the circumferential filament is attached to the third disintegrating matrix segment of each arm.

Embodiment 61. A method of treating an opioid abuse disorder in an individual, comprising administering the gastric residence system of any one of embodiments 36-60 to the individual.

Embodiment 62. A method of treating pain in an individual, comprising administering the gastric residence system of any one of embodiments 36-60 to the individual.

Embodiment 63. A gastric residence system comprising: a plurality of arms affixed to a central elastomer, wherein at least one arm comprises a drug-eluting component; each arm comprising a proximal end, a distal end, and an outer surface therebetween; wherein the proximal end of each arm is attached to the elastomer component and projects radially from the elastomer component, each arm having its distal end not attached to the elastomer component and located at a larger radial distance from the elastomer component than the proximal end; wherein the at least one arm comprising a drug eluting component comprises: a polymeric linker segment; a first disintegrating matrix segment attached to the polymeric linker segment; a first inert segment attached to the first disintegrating matrix segment; a second disintegrating matrix segment attached to the first inert segment; a second inert segment attached to the second disintegrating matrix segment; the drug-eluting component attached to the second inert segment, wherein the drug eluting component comprises a carrier polymer, buprenorphine or a salt thereof, and naloxone or a salt thereof, and wherein the drug eluting component further comprises a coating comprising a release rate-modulating polymer film; a third inert segment attached to the drug-eluting component; a third disintegrating matrix segment attached to the third inert segment; and a filament circumferentially connecting each arm.

Embodiment 64. A method of making a gastric residence system comprising: co-extruding at least one drug-eluting component comprising a carrier polymer, buprenorphine or a salt thereof, and naloxone or a salt thereof; and applying a rate-modulating release film to the at least one co-extruded drug-eluting component, wherein the gastric residence system is configured to be maintained within a stomach of a human body for at least 48 hours and to release buprenorphine for at least 48 hours, and the at least one co-extruded drug eluting component with the rate-modulating release film is configured to release at least 10% of the buprenorphine or the salt thereof after the first 24 hours of residence within the stomach and at least 10% of the naloxone or the salt thereof after the first 24 hours of residence within the stomach.

Embodiment 65. The method of embodiment 64, wherein the rate-modulating release film comprises polycaprolactone and copovidone.

Embodiment 66. The method of embodiment 65, wherein the rate-modulating release film comprises 60-90 wt % polycaprolactone.

Embodiment 67. The method of embodiment 65 or 66, wherein the rate-modulating release film comprises 10-40 wt % copovidone.

Embodiment 68. The method of any of embodiments 64-67, wherein the at least one co-extruded drug-eluting component comprises a first co-extruded portion comprising naloxone and a second co-extruded portion comprising buprenorphine and naloxone.

Embodiment 69. The method of embodiment 68, wherein co-extruding the at least one drug-eluting component comprises co-extruding the first co-extruded portion embedded in the second co-extruded portion.

Embodiment 70. The method of embodiment 68 or 69, wherein co-extruding the at least one drug-eluting component comprises co-extruding strands of the first co-extruded portion embedded within the second co-extruded portion.

Embodiment 71. The method of embodiment 68, wherein co-extruding the at least one drug-eluting component comprises co-extruding the first co-extruded portion layered on the second co-extruded portion.

Embodiment 72. The method of any of embodiments 68-71, wherein the first co-extruded portion is present at a ratio of 4:1 to the second co-extruded portion.

Embodiment 73. The method of any of embodiments 68-72, wherein the first co-extruded portion comprises 35-50 wt % buprenorphine.

Embodiment 74. The method of any of embodiments 68-73, wherein the first co-extruded portion comprises 2-7 wt % naloxone.

Embodiment 75. The method of any of embodiments 68-74, wherein the first co-extruded portion comprises 35-50 wt % polycaprolactone.

Embodiment 76. The method of any of embodiments 68-75, wherein the first co-extruded portion comprises polyethylene glycol and a poloxamer.

Embodiment 77. The method of any of embodiments 68-76, wherein the second co-extruded portion comprises 30-50 wt % naloxone.

Embodiment 78. The method of any of embodiments 68-77, wherein the second co-extruded portion comprises 50-60 wt % polycaprolactone.

Embodiment 79. The method of any of embodiments 68-78, wherein the second co-extruded portion comprises poloxamer.

Embodiment 80. The method of any of embodiments 64-79, wherein the at least one co-extruded drug-eluting component comprises 30 mg to 40 mg of buprenorphine or a salt thereof.

Embodiment 81. The method of any of embodiments 64-80, wherein the at least one co-extruded drug-eluting component comprises 8 mg to 15 mg of naloxone or a salt thereof.

Embodiment 82. The method of any of embodiments 64-81, wherein the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one co-extruded drug-eluting component.

Embodiment 83. The method of embodiment 82, wherein the plurality of arms comprises six arms.

Embodiment 84. The method of embodiment 82 or 83, wherein at least two arms of the plurality of arms comprises a co-extruded drug-eluting component of the at least one co-extruded drug-eluting component.

Embodiment 85. The method of embodiment 82 or 83, wherein at least three arms of the plurality of arms comprises a co-extruded drug-eluting component of the at least one co-extruded drug-eluting component.

Embodiment 86. The method of embodiment 82 or 83, wherein six arms of the plurality of arms comprises a co-extruded drug-eluting component of the at least one co-extruded drug-eluting component.

Embodiment 87. The method of any of embodiments 82-86, wherein each arm of the plurality of arms comprises a polymeric linker segment attached to the central elastomer, the polymeric linker segment comprising polycaprolactone.

Embodiment 88. The method of embodiment 87, wherein each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

Embodiment 89. The method of embodiment 88, wherein each arm of the plurality of arms comprises a first inert segment attached to the first disintegrating matrix segment, the first inert segment comprising polycaprolactone, and (BiO)₂CO₃.

Embodiment 90. The method of embodiment 89, wherein each arm of the plurality of arms comprises a second disintegrating matrix segment attached to the first inert segment, the second disintegrating matrix segment comprising polycaprolactone, hydroxypropyl methylcellulose acetate succinate, and a poloxamer.

Embodiment 91. The method of embodiment 90, wherein each arm of the plurality of arms comprises a second inert segment attached to the second disintegrating matrix segment, the second inert segment comprising polycaprolactone, and (BiO)₂CO₃.

Embodiment 92. The method of embodiment 91, wherein a first arm of the plurality of arms comprises a co-extruded drug-eluting component of the at least one co-extruded drug-eluting component attached to the second inert segment.

Embodiment 93. The method of embodiment 91, wherein a second arm of the plurality of arms comprises an inactive segment attached to the second inert segment, the inactive segment comprising polycaprolactone, copovidone, and a poloxamer.

Embodiment 94. The method of embodiment 92 or 93, wherein each arm of the plurality of arms comprises a third inert segment attached to one of the co-extruded drug-eluting component or the inactive segment, the third inert segment comprising polycaprolactone, and (BiO)₂CO₃.

Embodiment 95. The method of embodiment 94, wherein each arm of the plurality of arms comprises a third disintegrating matrix segment attached to the third inert segment, the third disintegrating matrix comprising polycaprolactone, hydroxypropyl methylcellulose acetate succinate (HPMCAS); stearic acid; and polypropylene glycol.

Embodiment 96. The method of embodiment 95, comprising a filament circumferentially connecting each arm, wherein the circumferential filament is attached to the third disintegrating matrix segment of each arm.

Embodiment 97. A method of making a gastric residence system comprising: extruding at least one drug-eluting component comprising a carrier polymer, buprenorphine or a salt thereof, and naloxone or a salt thereof; and applying a rate-modulating release film to the at least one drug-eluting component, wherein the gastric residence system is configured to be maintained within a stomach of a human body for at least 48 hours and to release buprenorphine for at least 48 hours, and the at least one drug-eluting component with the rate-modulating release film is configured to release at least 10% of the buprenorphine or the salt thereof after the first 24 hours of residence within the stomach and at least 10% of the naloxone or the salt thereof within the first 24 hours of residence within the stomach.

Embodiment 98. The method of embodiment 97, wherein the rate-modulating release film comprises polycaprolactone and copovidone.

Embodiment 99. The method of embodiment 98, wherein the rate-modulating release film comprises 60-90 wt % polycaprolactone.

Embodiment 100. The method of embodiment 98 or 99, wherein the rate-modulating release film comprises 10-40 wt % copovidone.

Embodiment 101. The method of any of embodiments 97-100, wherein the at least one drug-eluting component comprises 35-50 wt % buprenorphine.

Embodiment 102. The method of any of embodiments 97-101, wherein the at least one drug-eluting component comprises 2-7 wt % naloxone.

Embodiment 103. The method of any of embodiments 97-102, wherein the at least one drug-eluting component comprises 35-50 wt % polycaprolactone.

Embodiment 104. The method of any of embodiments 97-103, wherein the at least one drug-eluting component comprises polyethylene glycol and a poloxamer.

Embodiment 105. The method of any of embodiments 97-104, wherein the at least one drug-eluting component comprises 10 mg to 30 mg of buprenorphine or a salt thereof.

Embodiment 106. The method of any of embodiments 97-105, wherein the at least one drug-eluting component comprises 1 mg to 3 mg of naloxone or a salt thereof.

Embodiment 107. The method of any of embodiments 97-106, wherein the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one drug-eluting component.

Embodiment 108. The method of embodiment 107, wherein the plurality of arms comprises six arms.

Embodiment 109. The method of embodiment 107 or 108, wherein at least two arms of the plurality of arms comprises a drug-eluting component of the at least one drug-eluting component.

Embodiment 110. The method of embodiment 107 or 108, wherein at least three arms of the plurality of arms comprises a drug-eluting component of the at least one drug-eluting component.

Embodiment 111. The method of embodiment 107 or 108, wherein six arms of the plurality of arms comprises a drug-eluting component of the at least one drug-eluting component.

Embodiment 112. The method of any of embodiments 107-111, wherein each arm of the plurality of arms comprises a polymeric linker segment attached to the central elastomer, the polymeric linker segment comprising polycaprolactone.

Embodiment 113. The method of embodiment 112, wherein each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

Embodiment 114. The method of embodiment 113, wherein each arm of the plurality of arms comprises a first inert segment attached to the first disintegrating matrix segment, the first inert segment comprising polycaprolactone, and (BiO)₂CO₃.

Embodiment 115. The method of embodiment 114, wherein each arm of the plurality of arms comprises a second disintegrating matrix segment attached to the first inert segment, the second disintegrating matrix segment comprising polycaprolactone, hydroxypropyl methylcellulose acetate succinate, and a poloxamer.

Embodiment 116. The method of embodiment 115, wherein each arm of the plurality of arms comprises a second inert segment attached to the second disintegrating matrix segment, the second inert segment comprising polycaprolactone, and (BiO)₂CO₃.

Embodiment 117. The method of embodiment 116, wherein a first arm of the plurality of arms comprises a drug-eluting component of the at least one drug-eluting component attached to the second inert segment.

Embodiment 118. The method of embodiment 117, wherein a second arm of the plurality of arms comprises an inactive segment attached to the second inert segment, the inactive segment comprising polycaprolactone, copovidone, and a poloxamer.

Embodiment 119. The method of embodiment 117 or 118, wherein each arm of the plurality of arms comprises a third inert segment attached to one of the drug-eluting component or the inactive segment, the third inert segment comprising polycaprolactone, and (BiO)₂CO₃.

Embodiment 120. The method of embodiment 119, wherein each arm of the plurality of arms comprises a third disintegrating matrix segment attached to the third inert segment, the third disintegrating matrix comprising polycaprolactone, hydroxypropyl methylcellulose acetate succinate (HPMCAS); stearic acid; and polypropylene glycol.

Embodiment 121. The method of embodiment 120, comprising a filament circumferentially connecting each arm, wherein the circumferential filament is attached to the third disintegrating matrix segment of each arm.

The foregoing description sets forth exemplary systems, methods, techniques, parameters, and the like. It should be recognized, however, that such description is not intended as a limitation on the scope of the present disclosure but is instead provided as a description of exemplary embodiments.

Although the description herein uses terms first, second, etc. to describe various elements, these elements should not be limited by the terms. These terms are only used to distinguish one element from another.

1. A gastric residence system comprising:
at least one co-extruded drug-eluting component comprising a carrier polymer, buprenorphine or a salt thereof, and naloxone or a salt thereof; and
a rate-modulating release film coating the at least one co-extruded drug-eluting component,
wherein the gastric residence system is configured to be maintained within a stomach of a human body for at least 48 hours and to release buprenorphine for at least 48 hours, and the at least one co-extruded drug eluting component with the rate-modulating release film is configured to release at least 10% of the buprenorphine or the salt thereof after the first 24 hours of residence within the stomach and at least 10% of the naloxone or the salt thereof after the first 24 hours of residence within the stomach.

2. The gastric residence system of claim 1, wherein the rate-modulating release film comprises polycaprolactone and copovidone.

3. The gastric residence system of claim 2, wherein the rate-modulating release film comprises 60-90 wt % polycaprolactone.

4. The gastric residence system of claim 2 or 3, wherein the rate-modulating release film comprises 10-40 wt % copovidone.

5. The gastric residence system of any of claims 1-4, wherein the at least one co-extruded drug-eluting component comprises a first co-extruded portion comprising naloxone and a second co-extruded portion comprising buprenorphine and naloxone.

6. The gastric residence system of claim 5, wherein the first co-extruded portion is embedded in the second co-extruded portion.

7. The gastric residence system of claim 5 or 6, wherein the first co-extruded portion comprises one or more strands that are embedded within the second co-extruded portion.

8. The gastric residence system of claim 5, wherein the first co-extruded portion is layered on the second co-extruded portion.

9. The gastric residence system of any of claims 5-8, wherein the first co-extruded portion is present at a ratio of 4:1 to the second co-extruded portion.

10. The gastric residence system of any of claims 5-9, wherein the first co-extruded portion comprises 35-50 wt % buprenorphine.

11. The gastric residence system of any of claims 5-10, wherein the first co-extruded portion comprises 2-7 wt % naloxone.

12. The gastric residence system of any of claims 5-11, wherein the first co-extruded portion comprises 35-50 wt % polycaprolactone.

13. The gastric residence system of any of claims 5-12, wherein the first co-extruded portion comprises polyethylene glycol and a poloxamer.

14. The gastric residence system of any of claims 5-13, wherein the second co-extruded portion comprises 30-50 wt % naloxone.

15. The gastric residence system of any of claims 5-14, wherein the second co-extruded portion comprises 50-60 wt % polycaprolactone.

16. The gastric residence system of any of claims 5-15, wherein the second co-extruded portion comprises poloxamer.

17. The gastric residence system of any of claims 1-16, wherein the at least one co-extruded drug-eluting component comprises 30 mg to 40 mg of buprenorphine or a salt thereof.

18. The gastric residence system of any of claims 1-17, wherein the at least one co-extruded drug-eluting component comprises 8 mg to 15 mg of naloxone or a salt thereof.

19. The gastric residence system of any of claims 1-18, wherein the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one co-extruded drug-eluting component.

20. The gastric residence system of claim 19, wherein the plurality of arms comprises six arms.

21. The gastric residence system of claim 19 or 20, wherein at least two arms of the plurality of arms comprises a co-extruded drug-eluting component of the at least one co-extruded drug-eluting component.

22. The gastric residence system of claim 19 or 20, wherein at least three arms of the plurality of arms comprises a co-extruded drug-eluting component of the at least one co-extruded drug-eluting component.

23. The gastric residence system of claim 19 or 20, wherein six arms of the plurality of arms comprises a co-extruded drug-eluting component of the at least one co-extruded drug-eluting component.

24. The gastric residence system of any of claims 19-23, wherein each arm of the plurality of arms comprises a polymeric linker segment attached to the central elastomer, the polymeric linker segment comprising polycaprolactone.

25. The gastric residence system of claim 24, wherein each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

26. The gastric residence system of claim 25, wherein each arm of the plurality of arms comprises a first inert segment attached to the first disintegrating matrix segment, the first inert segment comprising polycaprolactone, and (BiO)₂CO₃.

27. The gastric residence system of claim 26, wherein each arm of the plurality of arms comprises a second disintegrating matrix segment attached to the first inert segment, the second disintegrating matrix segment comprising polycaprolactone, hydroxypropyl methylcellulose acetate succinate, and a poloxamer.

28. The gastric residence system of claim 27, wherein each arm of the plurality of arms comprises a second inert segment attached to the second disintegrating matrix segment, the second inert segment comprising polycaprolactone, and (BiO)₂CO₃.

29. The gastric residence system of claim 28, wherein a first arm of the plurality of arms comprises a co-extruded drug-eluting component of the at least one co-extruded drug-eluting component attached to the second inert segment.

30. The gastric residence system of claim 28, wherein a second arm of the plurality of arms comprises an inactive segment attached to the second inert segment, the inactive segment comprising polycaprolactone, copovidone, and a poloxamer.

31. The gastric residence system of claim 29 or 30, wherein each arm of the plurality of arms comprises a third inert segment attached to one of the co-extruded drug-eluting component or the inactive segment, the third inert segment comprising polycaprolactone, and (BiO)₂CO₃.

32. The gastric residence system of claim 31, wherein each arm of the plurality of arms comprises a third disintegrating matrix segment attached to the third inert segment, the third disintegrating matrix comprising polycaprolactone, hydroxypropyl methylcellulose acetate succinate (HPMCAS); stearic acid; and polypropylene glycol.

33. The gastric residence system of claim 32, comprising a filament circumferentially connecting each arm, wherein the circumferential filament is attached to the third disintegrating matrix segment of each arm.

34. A method of treating an opioid abuse disorder in an individual, comprising administering the gastric residence system of any one of claims 1-33 to the individual.

35. A method of treating pain in an individual, comprising administering the gastric residence system of any one of claims 1-33 to the individual.

36. A gastric residence system comprising:
at least one drug-eluting component comprising a carrier polymer, buprenorphine or a salt thereof, and naloxone or a salt thereof; and
a rate-modulating release film coating the at least one drug-eluting component,
wherein the gastric residence system is configured to be maintained within a stomach of a human body for at least 48 hours and to release buprenorphine for at least 48 hours, and the at least one drug-eluting component with the rate-modulating release film is configured to release at least 10% of the buprenorphine or the salt thereof after the first 24 hours of residence within the stomach and at least 10% of the naloxone or the salt thereof within the first 24 hours of residence within the stomach.

37. The gastric residence system of claim 36, wherein the rate-modulating release film comprises polycaprolactone and copovidone.

38. The gastric residence system of claim 37, wherein the rate-modulating release film comprises 60-90 wt % polycaprolactone.

39. The gastric residence system of claim 36 or 37, wherein the rate-modulating release film comprises 10-40 wt % copovidone.

40. The gastric residence system of any of claims 36-39, wherein the at least one drug-eluting component comprises 35-50 wt % buprenorphine.

41. The gastric residence system of any of claims 36-40, wherein the at least one drug-eluting component comprises 2-7 wt % naloxone.

42. The gastric residence system of any of claims 36-41, wherein the at least one drug-eluting component comprises 35-50 wt % polycaprolactone.

43. The gastric residence system of any of claims 36-42, wherein the at least one drug-eluting component comprises polyethylene glycol and a poloxamer.

44. The gastric residence system of any of claims 36-43, wherein the at least one drug-eluting component comprises 10 mg to 30 mg of buprenorphine or a salt thereof.

45. The gastric residence system of any of claims 36-44, wherein the at least one drug-eluting component comprises 1 mg to 3 mg of naloxone or a salt thereof.

46. The gastric residence system of any of claims 36-45, wherein the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one drug-eluting component.

47. The gastric residence system of claim 46, wherein the plurality of arms comprises six arms.

48. The gastric residence system of claim 46 or 47, wherein at least two arms of the plurality of arms comprises a drug-eluting component of the at least one drug-eluting component.

49. The gastric residence system of claim 46 or 47, wherein at least three arms of the plurality of arms comprises a drug-eluting component of the at least one drug-eluting component.

50. The gastric residence system of claim 46 or 47, wherein six arms of the plurality of arms comprises a drug-eluting component of the at least one drug-eluting component.

51. The gastric residence system of any of claims 46-50, wherein each arm of the plurality of arms comprises a polymeric linker segment attached to the central elastomer, the polymeric linker segment comprising polycaprolactone.

52. The gastric residence system of claim 51, wherein each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

53. The gastric residence system of claim 52, wherein each arm of the plurality of arms comprises a first inert segment attached to the first disintegrating matrix segment, the first inert segment comprising polycaprolactone, and (BiO)₂CO₃.

54. The gastric residence system of claim 53, wherein each arm of the plurality of arms comprises a second disintegrating matrix segment attached to the first inert segment, the second disintegrating matrix segment comprising polycaprolactone, hydroxypropyl methylcellulose acetate succinate, and a poloxamer.

55. The gastric residence system of claim 54, wherein each arm of the plurality of arms comprises a second inert segment attached to the second disintegrating matrix segment, the second inert segment comprising polycaprolactone, and (BiO)₂CO₃.

56. The gastric residence system of claim 55, wherein a first arm of the plurality of arms comprises a drug-eluting component of the at least one drug-eluting component attached to the second inert segment.

57. The gastric residence system of claim 55, wherein a second arm of the plurality of arms comprises an inactive segment attached to the second inert segment, the inactive segment comprising polycaprolactone, copovidone, and a poloxamer.

58. The gastric residence system of claim 56 or 57, wherein each arm of the plurality of arms comprises a third inert segment attached to one of the drug-eluting component or the inactive segment, the third inert segment comprising polycaprolactone, and (BiO)₂CO₃.

59. The gastric residence system of claim 58, wherein each arm of the plurality of arms comprises a third disintegrating matrix segment attached to the third inert segment, the third disintegrating matrix comprising polycaprolactone, hydroxypropyl methylcellulose acetate succinate (HPMCAS); stearic acid; and polypropylene glycol.

60. The gastric residence system of claim 59, comprising a filament circumferentially connecting each arm, wherein the circumferential filament is attached to the third disintegrating matrix segment of each arm.

61. A method of treating an opioid abuse disorder in an individual, comprising administering the gastric residence system of any one of claims 36-60 to the individual.

62. A method of treating pain in an individual, comprising administering the gastric residence system of any one of claims 36-60 to the individual.

63. A gastric residence system comprising:
a plurality of arms affixed to a central elastomer, wherein at least one arm comprises a drug-eluting component;
each arm comprising a proximal end, a distal end, and an outer surface therebetween; wherein the proximal end of each arm is attached to the elastomer component and projects radially from the elastomer component, each arm having its distal end not attached to the elastomer component and located at a larger radial distance from the elastomer component than the proximal end;
wherein the at least one arm comprising a drug eluting component comprises:
a polymeric linker segment;
a first disintegrating matrix segment attached to the polymeric linker segment;
a first inert segment attached to the first disintegrating matrix segment;
a second disintegrating matrix segment attached to the first inert segment;
a second inert segment attached to the second disintegrating matrix segment;
the drug-eluting component attached to the second inert segment, wherein the drug eluting component comprises a carrier polymer, buprenorphine or a salt thereof, and naloxone or a salt thereof, and wherein the drug eluting component further comprises a coating comprising a release rate-modulating polymer film;
a third inert segment attached to the drug-eluting component;
a third disintegrating matrix segment attached to the third inert segment;
and
a filament circumferentially connecting each arm.

64. A method of making a gastric residence system comprising:
co-extruding at least one drug-eluting component comprising a carrier polymer, buprenorphine or a salt thereof, and naloxone or a salt thereof; and
applying a rate-modulating release film to the at least one co-extruded drug-eluting component,
wherein the gastric residence system is configured to be maintained within a stomach of a human body for at least 48 hours and to release buprenorphine for at least 48 hours, and the at least one co-extruded drug eluting component with the rate-modulating release film is configured to release at least 10% of the buprenorphine or the salt thereof after the first 24 hours of residence within the stomach and at least 10% of the naloxone or the salt thereof after the first 24 hours of residence within the stomach.

65. The method of claim 64, wherein the rate-modulating release film comprises polycaprolactone and copovidone.

66. The method of claim 65, wherein the rate-modulating release film comprises 60-90 wt % polycaprolactone.

67. The method of claim 65 or 66, wherein the rate-modulating release film comprises 10-40 wt % copovidone.

68. The method of any of claims 64-67, wherein the at least one co-extruded drug-eluting component comprises a first co-extruded portion comprising naloxone and a second co-extruded portion comprising buprenorphine and naloxone.

69. The method of claim 68, wherein co-extruding the at least one drug-eluting component comprises co-extruding the first co-extruded portion embedded in the second co-extruded portion.

70. The method of claim 68 or 69, wherein co-extruding the at least one drug-eluting component comprises co-extruding strands of the first co-extruded portion embedded within the second co-extruded portion.

71. The method of claim 68, wherein co-extruding the at least one drug-eluting component comprises co-extruding the first co-extruded portion layered on the second co-extruded portion.

72. The method of any of claims 68-71, wherein the first co-extruded portion is present at a ratio of 4:1 to the second co-extruded portion.

73. The method of any of claims 68-72, wherein the first co-extruded portion comprises 35-50 wt % buprenorphine.

74. The method of any of claims 68-73, wherein the first co-extruded portion comprises 2-7 wt % naloxone.

75. The method of any of claims 68-74, wherein the first co-extruded portion comprises 35-50 wt % polycaprolactone.

76. The method of any of claims 68-75, wherein the first co-extruded portion comprises polyethylene glycol and a poloxamer.

77. The method of any of claims 68-76, wherein the second co-extruded portion comprises 30-50 wt % naloxone.

78. The method of any of claims 68-77, wherein the second co-extruded portion comprises 50-60 wt % polycaprolactone.

79. The method of any of claims 68-78, wherein the second co-extruded portion comprises poloxamer.

80. The method of any of claims 64-79, wherein the at least one co-extruded drug-eluting component comprises 30 mg to 40 mg of buprenorphine or a salt thereof.

81. The method of any of claims 64-80, wherein the at least one co-extruded drug-eluting component comprises 8 mg to 15 mg of naloxone or a salt thereof.

82. The method of any of claims 64-81, wherein the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one co-extruded drug-eluting component.

83. The method of claim 82, wherein the plurality of arms comprises six arms.

84. The method of claim 82 or 83, wherein at least two arms of the plurality of arms comprises a co-extruded drug-eluting component of the at least one co-extruded drug-eluting component.

85. The method of claim 82 or 83, wherein at least three arms of the plurality of arms comprises a co-extruded drug-eluting component of the at least one co-extruded drug-eluting component.

86. The method of claim 82 or 83, wherein six arms of the plurality of arms comprises a co-extruded drug-eluting component of the at least one co-extruded drug-eluting component.

87. The method of any of claims 82-86, wherein each arm of the plurality of arms comprises a polymeric linker segment attached to the central elastomer, the polymeric linker segment comprising polycaprolactone.

88. The method of claim 87, wherein each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

89. The method of claim 88, wherein each arm of the plurality of arms comprises a first inert segment attached to the first disintegrating matrix segment, the first inert segment comprising polycaprolactone, and (BiO)₂CO₃.

90. The method of claim 89, wherein each arm of the plurality of arms comprises a second disintegrating matrix segment attached to the first inert segment, the second disintegrating matrix segment comprising polycaprolactone, hydroxypropyl methylcellulose acetate succinate, and a poloxamer.

91. The method of claim 90, wherein each arm of the plurality of arms comprises a second inert segment attached to the second disintegrating matrix segment, the second inert segment comprising polycaprolactone, and (BiO)₂CO₃.

92. The method of claim 91, wherein a first arm of the plurality of arms comprises a co-extruded drug-eluting component of the at least one co-extruded drug-eluting component attached to the second inert segment.

93. The method of claim 91, wherein a second arm of the plurality of arms comprises an inactive segment attached to the second inert segment, the inactive segment comprising polycaprolactone, copovidone, and a poloxamer.

94. The method of claim 92 or 93, wherein each arm of the plurality of arms comprises a third inert segment attached to one of the co-extruded drug-eluting component or the inactive segment, the third inert segment comprising polycaprolactone, and (BiO)₂CO₃.

95. The method of claim 94, wherein each arm of the plurality of arms comprises a third disintegrating matrix segment attached to the third inert segment, the third disintegrating matrix comprising polycaprolactone, hydroxypropyl methylcellulose acetate succinate (HPMCAS); stearic acid; and polypropylene glycol.

96. The method of claim 95, comprising a filament circumferentially connecting each arm, wherein the circumferential filament is attached to the third disintegrating matrix segment of each arm.

97. A method of making a gastric residence system comprising:
extruding at least one drug-eluting component comprising a carrier polymer, buprenorphine or a salt thereof, and naloxone or a salt thereof; and
applying a rate-modulating release film to the at least one drug-eluting component,
wherein the gastric residence system is configured to be maintained within a stomach of a human body for at least 48 hours and to release buprenorphine for at least 48 hours, and the at least one drug-eluting component with the rate-modulating release film is configured to release at least 10% of the buprenorphine or the salt thereof after the first 24 hours of residence within the stomach and at least 10% of the naloxone or the salt thereof within the first 24 hours of residence within the stomach.

98. The method of claim 97, wherein the rate-modulating release film comprises polycaprolactone and copovidone.

99. The method of claim 98, wherein the rate-modulating release film comprises 60-90 wt % polycaprolactone.

100. The method of claim 98 or 99, wherein the rate-modulating release film comprises 10-40 wt % copovidone.

101. The method of any of claims 97-100, wherein the at least one drug-eluting component comprises 35-50 wt % buprenorphine.

102. The method of any of claims 97-101, wherein the at least one drug-eluting component comprises 2-7 wt % naloxone.

103. The method of any of claims 97-102, wherein the at least one drug-eluting component comprises 35-50 wt % polycaprolactone.

104. The method of any of claims 97-103, wherein the at least one drug-eluting component comprises polyethylene glycol and a poloxamer.

105. The method of any of claims 97-104, wherein the at least one drug-eluting component comprises 10 mg to 30 mg of buprenorphine or a salt thereof.

106. The method of any of claims 97-105, wherein the at least one drug-eluting component comprises 1 mg to 3 mg of naloxone or a salt thereof.

107. The method of any of claims 97-106, wherein the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one drug-eluting component.

108. The method of claim 107, wherein the plurality of arms comprises six arms.

109. The method of claim 107 or 108, wherein at least two arms of the plurality of arms comprises a drug-eluting component of the at least one drug-eluting component.

110. The method of claim 107 or 108, wherein at least three arms of the plurality of arms comprises a drug-eluting component of the at least one drug-eluting component.

111. The method of claim 107 or 108, wherein six arms of the plurality of arms comprises a drug-eluting component of the at least one drug-eluting component.

112. The method of any of claims 107-111, wherein each arm of the plurality of arms comprises a polymeric linker segment attached to the central elastomer, the polymeric linker segment comprising polycaprolactone.

113. The method of claim 112, wherein each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

114. The method of claim 113, wherein each arm of the plurality of arms comprises a first inert segment attached to the first disintegrating matrix segment, the first inert segment comprising polycaprolactone, and (BIO)₂CO₃.

115. The method of claim 114, wherein each arm of the plurality of arms comprises a second disintegrating matrix segment attached to the first inert segment, the second disintegrating matrix segment comprising polycaprolactone, hydroxypropyl methylcellulose acetate succinate, and a poloxamer.

116. The method of claim 115, wherein each arm of the plurality of arms comprises a second inert segment attached to the second disintegrating matrix segment, the second inert segment comprising polycaprolactone, and (BiO)₂CO₃.

117. The method of claim 116, wherein a first arm of the plurality of arms comprises a drug-eluting component of the at least one drug-eluting component attached to the second inert segment.

118. The method of claim 117, wherein a second arm of the plurality of arms comprises an inactive segment attached to the second inert segment, the inactive segment comprising polycaprolactone, copovidone, and a poloxamer.

119. The method of claim 117 or 118, wherein each arm of the plurality of arms comprises a third inert segment attached to one of the drug-eluting component or the inactive segment, the third inert segment comprising polycaprolactone, and (BiO)₂CO₃.

120. The method of claim 119, wherein each arm of the plurality of arms comprises a third disintegrating matrix segment attached to the third inert segment, the third disintegrating matrix comprising polycaprolactone, hydroxypropyl methylcellulose acetate succinate (HPMCAS); stearic acid; and polypropylene glycol.

121. The method of claim 120, comprising a filament circumferentially connecting each arm, wherein the circumferential filament is attached to the third disintegrating matrix segment of each arm.
